(19) United States
(12) Patent Application Publication
(10) Pub. No.: US 2008/0306074 A1
(43) Pub. Date: Dec. 11, 2008

(54) Compositions Comprising Addl Receptors, Related Compositions, and Related Methods

(76) Inventors: Pascale N. Lacor, Chicago, IL (US); Kirsten L. Viola, Chicago, IL (US); Mary P. Lambert, Glenview, IL (US); Yeusong Gong, Evanston, IL (US); Lei Chang, Westmont, IL (US); Pauline T. Velasco, Wilmette, IL (US); Eileen H. Bigio, Riverside, IL (US); Maria C. Buniel, Cleveland, OH (US); Sara J. Fernandez, Chicago, IL (US); Jasna Jerecic, San Francisco, CA (US); Susan Catalano, Hayward, CA (US); Todd Pray, San Mateo, CA (US); Ray Lowe, San Francisco, CA (US); Grant A. Krafft, Glenview, IL (US); William L. Klein, Winnetka, IL (US)

Correspondence Address: LICATA & TYRRELL P.C., 66 E. MAIN STREET, MARLTON, NJ, 08053, US

(21) Appl. No.: 11/569,122

(22) PCT Filed: May 16, 2005
(86) PCT No.: PCT/US05/17176

§ 371 Date: Mar. 7, 2007

Related U.S. Application Data
(60) Provisional application No. 60/571,267, filed on May 14, 2004.
(60) Provisional application No. 60/636,466, filed on Dec. 15, 2004.

(51) Int. Cl. A61K 31/498; G01N 33/567; A61P 25/28

(52) U.S. Cl. 514/249; 435/7.21

(57) ABSTRACT

Disclosed and claimed herein are compositions comprising ADDL receptors, related compositions, and related methods. ADDL receptors are typically, but perhaps not exclusively, localized at the post-synaptic density (PSD) of neuronal cells. Related compositions include, but are not limited to, compounds that affect, positively or negatively, ADDL binding to neuronal cells, either via one or more receptors localized at the post-synaptic density (PSD) or otherwise. Related methods include, but are not limited to, procedures to screen for compounds that affect, either positively or negatively, ADDL binding to neuronal cells, either via one or more receptors localized at the post-synaptic density (PSD) or otherwise. Other related methods include, but are not limited to, prevention and treatment of ADDL-related diseases, such as Alzheimer's disease, mild cognitive impairment, Down's syndrome, and the like, using compositions that inhibit, block, or otherwise interfere with ADDL binding to one or more receptors localized at the post-synaptic density of neuronal cells.

### STATEMENT OF GOVERNMENT SUPPORT

The invention described herein was made, in part, with support from the U.S. Department of Health and Human Services, National Institutes of Health (Grant Nos. NIH R01-AG18877, NIH R01-AG22547, and NIH R03-AG22237). Accordingly, the government may have certain rights in the invention. In addition, the invention was made, in part, with support from the Illinois Department of Public Health (ADRF Grant Nos. 33280010 and 43280003).

### BACKGROUND

1. Field

The invention relates to the fields of biology and medicine. Specifically, the invention relates to the prevention, diagnosis, and treatment of neurodegenerative diseases, including, but not limited to, ADDL-related diseases such as Alzheimer's disease, mild cognitive impairment, Down's syndrome, and the like.

2. Related Art

Alzheimer's disease (AD) is a progressive and degenerative dementia, characterized at autopsy by pathology hallmarks including, but not limited to, decreased brain mass, loss of particular sub-populations of neurons, and prevalence of senile plaques and neurofibrillary tangles (see e.g. Terry, R. D., et al. (1991) Ann. Neurol., vol. 30, pp. 572-580; Coyle, J. T. (1987) Alzheimer's Disease. In: Encyclopedia of Neuroscience (Adelman G, ed), pp 29-31. Boston-Basel-Stuttgart: Birkhäuser; references in either of the foregoing; and the like). In its early stages, however, AD manifests primarily as a profound inability to form new memories (see e.g. Selkoe, D. J. (2002) Science, vol. 298, pp. 789-791; references therein; and the like). The basis for this specific impact is not known, but evidence now favors involvement of neurotoxins derived from the amyloid beta (Aβ) peptide. Aβ is an amphipathic peptide and the abundance of its longer aggregation-prone 42 amino acid form is increased by gene mutations and risk factors linked to AD. Aβ 1-42 readily assembles into fibrils that deposit in AD brain tissue as amyloid plaques, one of the pathology hallmarks of AD. (The term “amyloid” is a generic label given to protein deposits with distinctive birefringent Congo red staining properties.) The prevalence of amyloid plaques and the in vitro neurotoxicity of Aβ 1-42 fibrils provided the central rationale for the original amyloid cascade hypothesis, which invoked deposition of fibrillar Aβ as the cause of neuron death and consequent memory loss and cognitive decline.

Despite its strong experimental support and intuitive appeal, the original amyloid cascade hypothesis has proven inconsistent with key observations, including the poor correlation between dementia and amyloid plaque burden (see e.g., Katzman, R. et al. (1988) Ann. Neurol., vol. 23, pp. 138-144; references therein; and the like). Particularly telling are recent studies of experimental AD vaccines done with transgenic hAPP mice (see e.g., Dodart, J. C. et al. (2002) Nat. Neurosci., vol. 5, pp. 452-457; Kotilinek, L. A. et al. (2002) J. Neurosci., vol. 22, pp. 6331-6335; references in either of the foregoing; and the like). These mice provide good models of early AD, developing age-dependent amyloid plaques and, most importantly, age-dependent memory dysfunction. Two surprising findings were obtained when mice were treated with monoclonal antibodies against Aβ: (1) Vaccinated mice showed reversal of memory loss, with recovery evident in 24 hours; (2) Cognitive benefits of vaccination accrued despite no change in plaque levels. Such findings are not consistent with a mechanism for memory loss dependent on neuron death caused by amyloid fibrils.

Salient flaws in the original hypothesis have been addressed in an updated hypothesis that incorporates central a role for non-fibrillar, neurologically active molecules formed by Aβ self-assembly (see e.g. Klein, W. L. et al. (2001) Trends Neurosci., vol. 24, pp. 219-224; references therein; and the like). Such molecules are termed ADDLs, which are soluble, neurotoxic assemblies of the 42 amino acid Aβ peptide. ADDLs are fundamentally different in structure from the insoluble Aβ fibrils found in AD-associated amyloid plaques (see e.g. Lambert, M. P. et al. (1998) Proc. Natl. Acad. Sci. USA, vol. 95, pp. 6448-6453; Chromy, B. A. et al. (2003) Biochemistry, vol. 42, pp. 12749-12760; references in either of the foregoing; and the like), and they provide a conceptual alternative to Aβ fibrils as the underlying cause of memory malfunction. In contrast to the non-specific cellular damage attributed to plaques, ADDLs trigger aberrant signaling in a specific subset of neurons, compromising memory function, far in advance of cell death (see e.g. Kirkitadze, M. D. et al. (2002) J, Neurosci, Res., vol. 69, pp. 567-577; Klein, W. L. et al. (2001) Trends Neurosci., vol. 24, pp. 219-224; references in either of the foregoing; and the like). Aβ1-42 oligomers are stable to SDS and form at low concentrations of Aβ42 (see e.g. Lambert, M. P. et al. (1998) Proc. Natl. Acad. Sci. USA, vol. 95, pp. 6448-6453; references therein; and the like). Essentially the missing links in the original cascade, ADDLs rapidly inhibit long-term potentiation (LTP), both in animals and in brain tissue slice cultures. LTP is a classic experimental paradigm for memory and synaptic plasticity. As such, ADDLs are specific neuropharmacologic ligands, the action of which should be reversible by appropriate therapeutic interventions, such as are the subject of this application. The updated ADDL hypothesis for AD posits that: (1) Memory loss stems from synapse failure, prior to neuron death; and (2) Synapse failure is caused by ADDLs, not fibrils (see e.g., Hardy, J. & Selkoe, D. J. (2002) Science, vol. 297; pp. 353-356; references therein; and the like). Support for this theory can be found in recent reports that soluble oligomers occur in brain tissue and are strikingly elevated in AD (see e.g., Kayed, R. et al. (2003) Science, vol. 300, pp. 486-489; Gong, Y. et al. (2003) Proc. Natl. Acad. Sci. USA, vol. 100, pp. 10417-10422; references in either of the foregoing; and the like) and in hAPP transgenic mice AD models (Kotilinek, L. A. et al. (2002) J. Neurosci., vol. 22, pp. 6331-6335; Chang, L. et al. (2003) J. Mol. Neurosci., vol. 20, pp. 305-313; references in either of the foregoing; and the like). ADDLs also are elevated in cerebrospinal fluid (CSF) of AD patients compared with levels in age-matched controls (Georganopoulou, D. G. et al. (2005) Proc. Natl. Acad. Sci. USA, vol. 102, no. 7, pp. 2273-2276; references therein; and the like).

Considerable interest now is focused on the mechanism by which ADDLs oligomers interact with neurons (see e.g., Caughey, B. & Lansbury, P. T. (2003) Annu. Rev. Neurosci., vol. 26, pp. 267-298; references therein; and the like). Previous theories have invoked membrane insertion or cytotoxic pore formation of Aβ monomer or oligomers, however these processes are non-specific and could not explain the highly selective pattern of compromised nerve cells observed in AD. Alternatively, ADDLs could bind as high-specificity ligands to particular membrane targets, thereby generating the highly selective synaptic pathology and the distinct pattern of symptoms observed in AD. In the current specification, results are presented to document highly specific binding interactions between ADDLs and subpopulations of cultured hippocampal neurons. These interactions appear to be identical for ADDLs extracted from AD brain tissue and from ADDLs prepared from synthetic Aβ 1-42 in vitro. The ADDL binding at cell surfaces manifests as small punctate clusters co-localized almost exclusively with a subpopulation of synaptic terminals. This highly specific synaptic binding is accompanied by ectopic induction of Arc, an immediate early gene, the over-expression of which has been linked to dysfunctional learning. It is possible that the selective targeting and functional disruption of particular synapses by ADDLs could underlie the specific loss of memory function in early AD and mild cognitive impairment. In this event, therapeutic interventions for these ADDL-related diseases should focus on agents that interfere with ADDL formation, ADDL signaling, or ADDL receptor binding, the subject of the current application.

This application is related to U.S. Pat. No. 6,218,506; International Patent App. Pub. No. WO 98/33815; U.S. Patent App. No. 60/086,582; U.S. patent application Ser. No. 09/369,236; International Patent App. No. PCT/US00/21458; U.S. patent application Ser. No. 09/745,057; U.S. patent application Ser. No. 10/166,856; International Patent App. No. PCT/US03/19640; U.S. Patent App. No. 60/095,264; U.S. Patent App. No. 60/415,074; U.S. patent application Ser. No. 10/676,871; U.S. patent application Ser. No. 10/924,372; International Patent App. No. PCT/US03/30930; U.S. Patent App. No. 60/568,449; U.S. Patent App. No. 60/571,267; U.S. Patent App. No. 60/584,695; U.S. Patent App. No. 60/621,776; U.S. Patent App. No. 60/636,466; U.S. Patent App. No. 2003/0068316; International Patent Publication No. WO 04/031400; International Patent Publication No. WO 01/10900; and International Patent Publication No. WO 98/33815; and the like.

### BRIEF SUMMARY

An embodiment of the invention disclosed and claimed herein comprised a composition of matter, wherein the composition comprises one or more receptors localized at neuronal post-synaptic densities, wherein the one or more receptors bind ADDLs. The one or more receptors can be synGAP, proSAP2/Shank3, a glutamate receptor, a kainate sub-type glutamate receptor, GluR6, an AMPA sub-type glutamate receptor, GluR2, mGluR1a, mGluR1b, mGluR1c, mGluR1d, mGluR5a, mGluR5b, a NMDA sub-type glutamate receptor, an integrin receptor, an adhesion receptor, NCAM, L1, cadherin, a trophic factor receptor, the fibroblast growth factor receptor 1, the fibroblast growth factor receptor 2, the TrkA receptor, the TrkB receptor, the erbB4 receptor, a close homolog of the erbB/EGF family of receptors, a receptor that binds trophins, the insulin receptor (IR), the insulin growth factor receptor 1 (IGF-1), a GABA receptor, sodium/potassium ATPase (Na⁺/K⁻ ATPase), CAM kinase II, the PrP protein, protein tyrosine phosphatase alpha (RPTPα) protein, a somatostatin receptor, a cannabinoid receptor, a sigma receptor, and/or the VIP/PACAL receptor. The invention also comprises any and all combinations of the foregoing receptors.

Another embodiment of the invention comprises a composition of matter, wherein the composition comprises one or more compounds that antagonize the binding of ADDLs to one or more receptors localized at the neuronal post-synaptic density. The invention further comprises a pharmaceutical preparation, wherein the preparation comprises one or more compounds that antagonize the binding of ADDLs to one or more receptors localized at the neuronal post-synaptic density. Another embodiment comprises a composition of matter, wherein the composition comprises one or more compounds that inhibit the binding of ADDLs to one or more receptors localized at the neuronal post-synaptic density. Another embodiment comprises a composition of matter, wherein the composition comprises one or more compounds that inhibit the binding of ADDLs to one or more receptors localized at the neuronal post-synaptic density, wherein the one or more compounds is CNQX.

Another embodiment of the invention comprises methods for treating an ADDL-related disease, wherein the method comprises the step of administering one or more compounds that antagonize the binding of ADDLs to one or more receptors localized at the neuronal post-synaptic density. In particular, wherein the ADDL-related disease comprises or includes, but is not limited to, Alzheimer's disease (AD), mild cognitive impairment (MC1), Down's syndrome, and the like. Another embodiment of the invention comprises methods for treating an ADDL-related disease, wherein the method comprises the step of administering one or more compounds that antagonize the binding of ADDLs to one or more receptors localized at the neuronal post-synaptic density, wherein the one or more compounds is CNQX or a pharmaceutically acceptable derivative of CNQX. Another embodiment of the invention comprises methods for treating an ADDL-related disease, wherein the method comprises the step of administering one or more compounds that antagonize the binding of ADDLs to one or more receptors localized at the neuronal post-synaptic density, wherein the one or more receptors is synGAP, proSAP2/Shank3, a glutamate receptor, a kainate sub-type glutamate receptor, GluR6, an AMPA sub-type glutamate receptor, GluR2, mGluR1a, mGluR1b, mGluR1c, mGluR1d, mGluR5a, mGluR5b, a NMDA sub-type glutamate receptor, an integrin receptor, an adhesion receptor, NCAM, L1, cadherin, a trophic factor receptor, the fibroblast growth factor receptor 1, the fibroblast growth factor receptor 2, the TrkA receptor, the TrkB receptor, the erbB4 receptor, a close homolog of the erbB/EGF family of receptors, a receptor that binds trophins, the insulin receptor (IR), the insulin growth factor receptor 1 (IGF-1), a GABA receptor, sodium/potassium ATPase (Na⁺/K⁻ ATPase), CAM kinase II, the PrP protein, the receptor protein tyrosine phosphatase alpha (RPTPα) protein, a somatostatin receptor, a cannabinoid receptor, a sigma receptor, and/or the VIP/PACAL receptor. The invention further comprises such methods comprising any and all combinations of these receptors.

The invention further comprises a composition of matter, wherein the composition comprises a biotin-labeled ADDL. In particular, wherein the composition comprises an ADDL containing one or more biotin moieties. More particularly, wherein the composition comprises an ADDL containing one or more epitopes recognized by an antibody. In particular, wherein the epitope is a peptide sequence. More in particular, wherein the epitope is a small organic molecule.

The invention further comprises a composition of matter, wherein the composition of matter is an ADDL surrogate containing one or more biotin moieties.

The invention further comprises a composition of matter, wherein the composition of matter is an ADDL surrogate containing one or more biotin moieties, and wherein the surrogate comprises a peptide or peptide mimic containing specific structural elements that enable formation of an internal beta sheet, the formation of which enables assembly into oligomers, wherein the oligomers are capable of binding to an ADDL receptor localized at the neuronal post-synaptic density.

The invention further comprises a composition of matter, wherein the composition of matter is an ADDL surrogate containing one or more biotin moieties, and wherein the composition comprises a peptide or peptide mimic containing specific structural elements that enable the formation of an internal C-terminal beta sheet, the formation of which enables assembly into oligomers, wherein the oligomers are capable of binding to an ADDL receptor localized at the neuronal post-synaptic density.

The invention further comprises a composition of matter, wherein the composition of matter is an ADDL surrogate containing one or more biotin moieties, and wherein the composition comprises a peptide or peptide mimic containing the motif:

```
R-XXXX
            Z
    XXXX
```

wherein Z is glycyl glycyl, prolyl-glycyl, glycyl-prolyl, or any other dipeptide or dipeptide mimic capable of forming a beta-turn, or any other beta-turn mimic, and where X is any amino acid or amino acid mimic, the presence of which enables the formation of an internal beta sheet, the formation of which enables assembly into oligomers, wherein the oligomers are capable of binding to an ADDL receptor localized at the neuronal post-synaptic density.

The invention further comprises a composition of matter, wherein the composition of matter is an ADDL surrogate containing one or more biotin moieties, and wherein the composition comprises a dipeptide-functionalized beta turn mimic capable of assembling into oligomers, wherein the oligomers are capable of binding to an ADDL receptor localized at the neuronal post-synaptic density.

The invention further comprises a composition of matter, wherein the composition of matter is an ADDL surrogate containing one or more biotin moieties, and wherein the composition comprises the peptide sequence:

DSGYEVUUQKLVFFAEDVGSNKGAIIGLMVGGAIVV

wherein U is any hydrophilic amino acid residue other than histidine, wherein the peptide is capable of assembling into oligomers, wherein the oligomers are capable of binding to an ADDL receptor localized at the neuronal post-synaptic density.

The invention further comprises a composition of matter, wherein the composition of matter is an ADDL surrogate containing one or more biotin moieties, and wherein said composition comprises the peptide sequence:

DSGYEVUUQKLVFFAEDVGSNKGAIIGLMVGGXAIVV

wherein U is any hydrophilic amino acid residue other than histidine, wherein X is any hydrophobic amino acid, wherein said peptide is capable of assembling into oligomers, wherein said oligomers are capable of binding to an ADDL receptor localized at the neuronal post-synaptic density.

The invention further comprises a composition of matter, wherein the composition of matter is an ADDL surrogate containing one or more biotin moieties, and wherein said composition comprises the peptide sequence:

RUUQKLVFFAEDVGSNKGAIIGLMVGGAIVV

wherein R is any peptide, U is any hydrophilic amino acid residue other than histidine, wherein said peptide is capable of assembling into oligomers, wherein said oligomers are capable of binding to an ADDL receptor localized at the neuronal post-synaptic density.

The invention further comprises a composition of matter, wherein the composition of matter is an ADDL surrogate containing one or more biotin moieties, and wherein said composition comprises the peptide sequence:

RUUQKLVFFAEDVGSNKGAIIGLMVGGXAIVV

wherein R is any peptide, where U is any hydrophilic amino acid residue other than histidine, where X is any hydrophobic amino acid, wherein said peptide is capable of assembling into oligomers, wherein said oligomers are capable of binding to an ADDL receptor localized at the neuronal post-synaptic density.

The invention further comprises a composition of matter, wherein the composition comprises a fluorescently-labeled ADDL. In particular, wherein the fluorescent label is fluorescein, tetramethylrhodamine, and/or an Alexa™ dye.

The invention further comprises a method of screening for compounds that interfere with the binding of ADDLs to one or more receptors localized at a post-synaptic density, wherein the method comprises the steps of: a) generating ADDLs; b) adding the ADDLs generated in step a) to tissue culture cells that comprise the post-synaptic density in the presence of one or more compounds suspected of interfering with the binding of the ADDLs to the one or more receptors localized at the post-synaptic density; and c) measuring the effect or effects of the one or more compounds on the binding of the ADDLs to the one or more receptors localized at the post-synaptic density. In particular, wherein the ADDLs are biotin-labeled ADDLs, fluorescently-labeled ADDLs, or combinations of biotin-labeled ADDLs and fluorescently-labeled ADDLs. More in particular, wherein the measuring (or detecting or detection) utilizes an antibody that recognizes ADDLs when bound to one or more of the receptors localized at the neuronal post-synaptic density. Even more in particular, wherein the measuring (or detecting or detection) utilizes avidin or streptavidin that recognizes the biotin within a biotin-labeled ADDL, when said composition is bound one or more of the receptors localized at the neuronal post-synaptic density. Also in particular, wherein the measuring (or detecting or detection) measures the amount of fluorescence associated with a fluorescent-labeled secondary antibody that recognizes the anti-ADDL antibody. In particular, wherein the measuring (or detecting or detection) measures a fluorescent or luminescent signal generated by an enzyme-antibody or enzyme-streptavidin conjugate.

Another embodiment of the invention comprises a method of identifying compounds that interfere with the binding of ADDL surrogates to one or more receptors localized at a post-synaptic density, wherein the method comprises the steps of: a) generating ADDL surrogates; b) adding the ADDL surrogates generated in step a) to tissue culture cells that comprise the post-synaptic density in the presence of one or more compounds suspected of interfering with the binding of the ADDL surrogates to the one or more receptors localized at the post-synaptic density; and c) measuring the effect or effects of the one or more compounds on the binding of the ADDLs to the one or more receptors localized at the post-synaptic density.

Another embodiment of the invention comprises a method of identifying compounds that interfere with the binding of ADDL surrogates to one or more receptors localized at a post-synaptic density, wherein the method comprises the steps of: a) generating ADDL surrogates; b) adding the ADDL surrogates generated in step a) to tissue culture cells that comprise the post-synaptic density in the presence of one or more compounds suspected of interfering with the binding of the ADDL surrogates to the one or more receptors localized at the post-synaptic density; and c) measuring the amount of arc protein that is produced within the neurons using an anti-arc antibody.

Another embodiment of the invention comprises a method to measure ADDL binding to a post-synaptic density, wherein the method comprises the steps of: a) generating ADDLs; b) adding the ADDLs generated in step a) to tissue culture cells that comprise the post-synaptic density; and c) measuring the punctate binding that is characteristic of ADDL binding to the post-synaptic density.

Another embodiment of the invention comprises a method of identifying compounds that interfere with ADDL binding to a post-synaptic density, wherein the method comprises the steps of: a) generating ADDLs; b) adding the ADDLs generated in step a) to tissue culture cells that comprise the post-synaptic density, in the presence of one or more compounds suspected of interfering with ADDL binding to the post-synaptic density; and c) measuring the effects of the one or more compounds on the punctate binding that is characteristic of ADDL binding to the post-synaptic density.

Another embodiment comprises ADDL binding proteins that mediate the interactions between ADDLs and post-synaptic dendritic spines. One embodiment of the invention is the synGAP protein which binds ADDLs, and an amino acid sequence shared by synGAP and glutamate receptors. The sequence can comprise the amino acids FEGYIDLGRELSTLHALLWEVLPQLSKEALL (SEQ ID No. ______), or an active fragment thereof, of synGAP. The sequence can comprise the amino acids YEGYCVDLATEIAKHCGFKYKLTIVGDGKYGA (SEQ ID No. ______), or an active fragment thereof, of GluR2. The sequence can comprise the amino acids FEGYCLDLLKELSNILGFIYDVKLVPDGKYGA (SEQ ID No. ______), or an active fragment thereof, of GluR5. The sequence can comprise the amino acids FEGYCIDLLRELSTILGFTYEIRLVEDGKYGA (SEQ ID No. ______), or an active fragment thereof, of GluR6. The sequence can comprise other sequences that are 95% homologous to one or more of these sequences, 90% homologous to one or more of these sequences, 85% homologous to one or more of these sequences, 80% homologous to one or more of these sequences, 75% homologous to one or more of these sequences, and 70% homologous to one or more of these sequences.

Another embodiment of the invention is an ADDL receptor complex comprising one or more glutamate receptors and one or more post-synaptic density (PSD) scaffolding proteins, including but not limited to, proSAP2/shank3. When ADDLs bind to such a receptor complex, glutamate receptor signaling is activated, resulting in blockage of LTP.

A further embodiment of this invention comprises antagonist molecules that abrogate ADDL binding to the ADDL receptor complex and prevent ADDL blockage of LTP. Additional embodiments of this invention comprise methods for discovery of anti-ADDL compounds and methods of use of anti-ADDL compounds to treat ADDL-related diseases such as Alzheimer's disease, mild cognitive impairment, ischemia and stroke induced dementia, and Down's syndrome.

### BRIEF DESCRIPTION OF THE DRAWINGS

FIG. 1: Aβ oligomers are deposited extracellularly around neurons and are highly elevated in Alzheimer's disease cortex. Sections from frontal cortex of AD brain were immunolabeled for ADDLs using M94 antibody and visualized with either fluorescent- or peroxidase-conjugated secondary antibodies (A and B, respectively). Note the diffuse synaptic-type labeling surrounding the cell body of a single pyramidal neuron in both labeling conditions (arrows). No IR deposits are observed in non-demented controls (not shown). Scale bar represents 10 μm in images A and B. Shown in (C) is a scatter plot of soluble Aβ levels measured by dot blot from two brain regions [cortex (square) and cerebellum (triangle)] form 9 subjects with AD (filled symbols) or 15 subjects without AD diagnosis (open symbols). Brain samples were assayed by dot blot (insert) and analyzed by densitometry. Each point is the relative intensity average of duplicate measurements. The bar indicates the mean of each group (AD cortex: 2.281+/−0.202; CTL cortex: 0.206+/−0.083; AD cerebellum: 0.263+/−0.090 and CTL cerebellum: 0.097+/−0.013; values represent mean+/−SEM). This plot shows that the scatter of Aβ levels in AD cortex is larger than in control cortex, as well as being significantly elevated over control (p<0.0001, AD vs CTL). However, for the cerebellum, the difference between AD and CTL Aβ levels were not as pronounced and not quite significant (p=0.1316, AD vs CTL). Mann-Whitney test was used and a one-tailed p value was established to test for significance using GraphPad InStat 3 software.

FIG. 2: Aβ oligomers (ADDLs) from AD brain bind neurons with punctate specificity. Primary hippocampal neurons were incubated for 30 minutes with soluble extracts from AD frontal cortex (A) or AD CSF (E). Some cultures were incubated with age-matched control cortical extract (B) or CSF (F). In some experiments, hippocampal neurons were incubated similarly with Centricon-fractionated soluble AD extract (see methods) containing species with mass between 10 and 100 kDa (C) or species with mass ≦10 kDa (D). Unbound species were washed out and ADDL attachment was assessed under non-permeabilized immunolabeling conditions using the rabbit polyclonal oligomer-specific M94 antibody (as described by (Lambert et al., 2001). Soluble extracts from AD brain (A) and CSF (E) contain ADDLs which bind selectively to neuronal surfaces with a punctate distribution. No labeling was detected with cortical extract (B) and CSF (F) from age-matched controls. Centricon™ filter fractionation of AD extracts containing species with a mass ranging from 10 to 100 kDa showed punctuate staining indistinguishable from unfractionated soluble extract, while binding species were not present in the fraction containing species with mass ≦10 kD. Similar observations were obtained from three independent experiments.

FIG. 3: Synthetic ADDLs, but not low molecular weight species, bind neurons analogously to AD-derived species. Primary hippocampal neurons were incubated with synthetic ADDLs fractionated by a Centricon™ filter (A,B) or biotinylated-ADDLs separated by size exclusion chromatography (E,F) for 30 min (as described in (Chromy et al., 2003). After removing unbound species by washing with fresh media, cell-bound ADDLs were assessed under non-permeabilized immunolabeling conditions using M94 and Alexa-488 conjugated anti-rabbit secondary antibody (A, B) or Alexa488-conjugated streptavidin (E,F). Confocal images demonstrate that oligomer immunoreactivity is at the plasma membrane of neurons and predominantly within dendritic arbors, although some binding to cell bodies is also evident. Punctate binding is reminiscent of receptor clusters and analogous to that of Alzheimer's Aβ oligomers (FIG. 2). As with fractionated soluble oligomers from AD brain, incubation of hippocampal neurons with synthetic ADDLs species ranging from 100-10 kD shows immunoreactive puncta (A), while species ≦10 kDa, which would contain monomer and dimer, do not (B). (Insert) Western blot of ADDL species present in the culture media after a 6 hour incubation with hippocampal neurons demonstrated that ADDLs are stable and contain no species with molecular weight higher than 100 kDa (C). Lanes represent two separate culture media. Separation of biotinylated ADDLs (˜14 nmol of a 70 μM ADDL preparation) by size-exclusion chromatography on Superdex 75 yielded 2 peaks (D). Histogram of elution volume vs absorbance at 280 nm depicted Peak 1 at 8.1 ml with an absorbance at 16.9 mAU and Peak 2 at 13.9 ml with an absorbance at 11.7 mAU. Fractions B1 and D6 with respective protein concentration of 6.5 μM and 4 μM were incubated for 1 hour with mature hippocampal cells at a final concentration of 500 nM in parallel to a SEC-control fraction (taken between peaks 1 and 2). Binding of biotinylated species was detected with Alexa-Fluor 488 conjugated streptavidin. Hot spots of fluorescence were observed exclusively with peak 1 fraction B1 (E), consistent with species of molecular weight over 50 kDa such as 12-mers. No fluorescence was seen with the peak 2 fraction D6 (F) or the control fraction (not shown). Confocal images were acquired with constant confocal microscope settings (laser power, filters, detector gain, amplification gain, and amplification offset). Images are representative of 3 different experiments. Scale bar represents 40 μm.

FIG. 4: ADDL binding is cell-specific. Double-labeling immunofluorescence studies were performed on mature hippocampal neurons (21 DIV) with mouse monoclonal anti-αCaM kinase II and rabbit polyclonal anti-ADDL (M94), and visualized with Alexa Fluor 594 (red) and Alexa Fluor 488 (green) secondary antibodies, respectively (A,B). Similar double labeling experiments were conducted with mouse monoclonal anti-PSD-95 (C, red) and anti-ADDL (green). Overlays (B, C) of three-dimensional reconstructed images of confocal z-series scans (taken at 0.5 μm steps) show that ADDLs bind selectively to some αCaM kinase II positive neurons (overlap appears yellow), depicted here after 6 hr incubation with ADDLs. Similar cell selectivity was observed with the PSD-95 labeling. Note that only one of the two neurons is targeted by ADDLs. Similar cell-specific binding and colocalization between ADDLs and αCaM kinase II or PSD-95 positive neurons was observed after 30 min ADDLs incubation (not shown). Scale bar represents 20 μm.

FIG. 5: ADDLs specifically target a subset of PSD-95-positive terminals. Hippocampal neurons treated with ADDLs were double immunolabeled for PSD-95 (red, A) and ADDLs (green, B). Overlaying the confocal reconstructed z-series scans shows that dendritic clusters of ADDL-IR puncta almost completely colocalized with PSD-95, as seen by the amount of yellow puncta in the merged image (C). The degree of colocalization between ADDLs and PSD-95 was quantified using Metamorph software. Bar graphs show the number of PSD-95 sites targeted by ADDLs (E) and the number of ADDL binding sites colocalized with PSD-95 (F) for 14 different fields (40× objective). Graph (E) shows many PSD-95 sites are unoccupied by ADDLs (yellow bar: PSD-95 colocalized with ADDLs; red bar: PSD-95 without ADDLs) (mean total oligomer binding sites per field was 1062+/−125; mean oligomer sites that colocalized with PSD-95 was 971+/−105). Graph (F) shows for each field the proportion of ADDL puncta localized to PSD-95 synaptic sites. The number of ADDLs at PSD-95 sites (yellow bar) greatly exceeds ADDLs at non-synaptic sites (green bar). Pie charts illustrate the distribution summed for all fields, showing the fraction of PSD-95 sites occupied by ADDLs (G) and the fraction of ADDL binding sites co-localized with PSD-95 (H). Results are presented as an average percentage+/−SEM for all 14 different fields (mean total sites per field was 1960+/−174, with 50+/−2% not associated with oligomer binding sites). In summary, half of PSD-95 puncta are targeted by ADDLs (G), while over 90% ADDL puncta colocalize with PSD-95 (H). Analyses verify that ADDLs specifically localize to a subset of synaptic sites. Scale bar represents 10 μm. (Insert, D) Image of hippocampal neurons showing ADDLs (green) juxtaposed to pre-synaptic marker, synaptophysin (red), rather than superimposed, as above.

FIG. 6: Localization of ADDL binding sites to dendritic spines. Highly differentiated hippocampal cells (21 DIV) treated with synthetic ADDLs for 1 hr were double-immunolabeled for ADDLs (green) and αCaM kinase II (red). An ADDL-bound αCaM kinase II-positive neuron is pictured (A). Higher magnification illustrates that many of the ADDL-IR puncta co-distributed with αCaM kinase II-enriched dendritic spines (B). As pointed by arrows, ADDLs mainly targeted dendritic spines. Image is representative of three separate trials. Scale bars represent 40 μm (A) and 8 μm (B).

FIG. 7: Rapid ADDL-induced synaptic expression of the immediate early gene Arc. Hippocampal neurons were exposed to synthetic ADDLs for 5 minutes and then labeled for Arc (red) and ADDLs (green). Merging the images shows points of colocalization between ADDL puncta and Arc-positive synapses (yellow). Scale bar represents 8 μm.

FIG. 8: ADDLs promote sustained upregulation of Arc. Hippocampal cells treated with vehicle (A, C) or ADDLs (B, D) for 1 hour (A,B) or 6 hours (C,D) were fixed, permeabilized and labeled for Arc protein. Immunofluorescence demonstrated a large ADDL-induced increase in Arc expression throughout the dendrites and dendritic spines of a subset of neurons; in vehicle-treated cells, Arc expression is restricted to the neuronal cell body. Insert represents extracts from hippocampal cells treated with vehicle (−) or ADDLs (+) for 1 hour immunoblotted after SDS-PAGE using an Arc polyclonal antibody. Immunoblots show increased concentration of Arc in ADDL-treated (+) compared to vehicle-treated (−) cell extracts. Cylophilin B (cyclo) was used to normalize with respect to protein loading; ratio of Arc/cyclo IR was 0.70+/−0.11 in controls and 3.51+/−0.76 in oligomer-treated samples (n=4, p=0.01 using t-test: paired two-sample for means). Blot is representative of 4 independent experiments. Hippocampal cells also were treated for 6 hours with vehicle (C) or ADDLs (D) and permeabilized and labeled for Arc. High magnification confocal images of vehicle-treated neurons show that Arc-IR puncta were localized to the dendritic shaft with only a few spine heads reaching an intensity level higher than that found in the dendritic shaft. The dendrites of ADDL-treated neurons show a punctate pattern of intense Arc-IR concentrated in dendritic spine heads as well as upregulated Arc-IR throughout the dendritic shafts and spines. Controls for the primary antibodies showed no labeling. Identical results were obtained in three independent trials. Scale bars represent 20 μm (A,B) and 6 μm (C,D).

FIG. 9: Hypothetical role for Arc in ADDL-induced synapse failure and memory loss. See Example 1 Discussion section.

FIG. 10: Confocal immunofluorescence image representing the fluorescence distribution of Aβ soluble oligomers (green) on the dendritic branches of a mature Ca++/calmodulin-dependent protein kinase II alpha (CaMKII^{α}) positive hippocampal neuron (pink-red). Aβ soluble oligomers specifically targeted dendritic spines which highly expressed CaMKII^{α} (overlap is yellow). Box represents a magnification of dendritic spines.

FIGS. 11-1 & 11-2: Receptor—ADDL Apposition and Co-localization.

FIG. 12: NR2B membrane expression is decreased after ADDL exposure.

FIG. 13: Time-course treatment of hippocampal neurons with ADDLs results in a temporal post-synaptic response monitored by spinophilin immunofluorescence (IF) intensity and spine morphology.

FIG. 14: Erb-B4 IF staining intensity is increased after 1 hr of ADDL exposure.

FIG. 15 (panels A & B): ADDLs bind to post-synaptic densities (PSDs) and not active zones in an ELISA assay.

FIG. 16 (panels A & B): CNQX blocks binding of ADDLs to synaptosomes. CNQX decreases the amount of ADDLs bound to synaptosomes.

FIG. 17 (Panels A & B): CNQX blocks ADDL binding to synaptosomes. CNQX decreases PSD 95 in ADDL immunoprecipitation (IP).

FIG. 18: CNQX blocks the binding of ADDLs to the surface of neurons.

FIG. 19: Panel A: Object identification using compartmental analysis. Channel 1 depicts nuclear staining (DAPI), channel 2 depicts neuronal staining using MAP2 antibody, and Channel 3 depicts ADDL staining using an anti ADDL antibody. Only neurons contain both a DAPI positive nucleus and a MAP2 positive cell body. The average intensity of ADDL binding to the proximal dendrites is measured in neurons (green pixels in Channel 3). Panel B: Quantification of ADDL binding to primary hippocampal neurons. Neurons exposed to ADDLs in wells 1-10, 13-22, 25-34 and 37-42 have a much higher percentage of neurons with very intense ADDL binding than vehicle control wells (all other wells).

FIG. 20: ADDL binding to primary hippocampal neurons. Detection of increasing amounts of biotinylated ADDLs bound to neuronal cells using alkaline phosphatase coupled to streptavidin.

FIG. 21A-21C: A ClustalW sequence alignment of human synGAP, human GluR2 precursor, and human GluR6 precursor according to standard procedures.

FIG. 22: Glutamate and glutamate receptor ligands CNQX and NS-102 block ADDL binding to dendritic receptors.

FIG. 23: An immunofluorescence examination of the effects of glutamate receptor (GluR) blockers on ADDL binding to hippocampal cells.

FIG. 24: Glutamate blocks ADDL binding to synaptosomes in panning assay.

FIG. 25: Synaptosome panning shows that ADDL binding is dependent on synaptosome concentration.

FIG. 26: Using cholera toxin subunit B to immobilize synaptosomes shows ADDL and synaptosome concentration dependent binding.

FIG. 27: ADDL-synaptosome immunoprecipitation. Synaptosomes were incubated with ADDLs or vehicle in F12/FBS (F12 media, 5% FBS). Treated-synaptosomes were immunoprecipitated using magnetic beads coated with an anti-ADDL monoclonal antibody (Dyna-20C2) in F12/FBS. The presence of synaptic markers was assessed in different fractions using an anti-PSD95 antibody in a standard Western blots.

FIG. 28: ADDLs bind to PSD and not to Active Zones of cortical synaptosomes.

FIG. 29: Characterization of biotin labeled ADDLs. Biotinylated ADDLs (b-ADDLs) appear in low molecular weight (LMW) peak.

FIG. 30: Panel A: ADDLs from a mixture of biotinylated and unlabeled AbI-42 were fractionated on SEC (ADDL31, top left panel) and peak fractions analyzed by native-PAGE Western blot with a probe for the biotin label (top right panel). Panel B: ADDLs from a mixture of biotinylated and unlabeled Abl-42 were subjected to SDS-PAGE and analyzed by silver stain (bottom left panel) and Western blot (bottom right panels). Blots were probed for biotin or immunostained with monoclonal antibodies (6E10 and 20C2). Unlabeled ADDLs were included for comparison.

### DETAILED DESCRIPTION

Conventional laboratory techniques and procedures can be generally performed according to methods well known in the art and as described in various general and more specific references that are cited and discussed throughout the present specification. See e.g., Sambrook et al. (2001, MOLECULAR CLONING: A LABORATORY MANUAL, 3d ed., Cold Spring Harbor Laboratory Press, Cold Spring Harbor, N.Y.), which is incorporated herein by reference for any purpose. Unless specific definitions are provided, the nomenclature utilized in connection with, and the laboratory procedures and techniques of, molecular biology, genetic engineering, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

In certain embodiments, the invention provides pharmaceutical compositions comprising a therapeutically effective amount, or dose, of a compound that inhibits ADDL binding to neuronal post synaptic densities. As is well known in the art, such compositions can be prepared together with a pharmaceutically acceptable diluent, carrier, solubilizer, emulsifier, preservative, and/or adjuvant.

As used herein, the term “agent” denotes a chemical compound, a mixture of chemical compounds, a biological macromolecule, or an extract made from biological materials.

As used herein, the term “pharmaceutical composition” refers to a composition comprising a pharmaceutically acceptable carrier, excipient, or diluent and a chemical compound, peptide, or composition as described herein that is capable of inducing a desired therapeutic effect when properly administered to a patient.

As used herein, the term “therapeutically effective amount” refers to the amount of a pharmaceutical composition of the invention or a compound identified in a screening method of the invention determined to produce a therapeutic response in a mammal. Such therapeutically effective amounts are readily ascertained by one of ordinary skill in the art and using methods as described herein.

As used herein, the term “substantially pure” means an object species that is the predominant species present (i.e., on a molar basis it is more abundant than any other individual species in the composition). In certain embodiments, a substantially purified fraction is a composition wherein the object species comprises at least about 50 percent (on a molar basis or on a weight or number basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (wherein contaminating species cannot be detected in the composition by conventional detection methods) wherein the composition consists essentially of a single macromolecular species.

As used herein, the term “patient” includes human and animal subjects.

Unless otherwise required by context, singular terms shall include pluralities and plural terms shall include the singular.

Routes of administration contemplated herein may be by any systemic means including oral, intraperitoneal, subcutaneous, intravenous, intramuscular, transdermal, inhalation or by other routes of administration. Osmotic mini-pumps and timed-released pellets or other depot forms of administration may also be used. Acceptable formulation materials preferably are nontoxic to recipients at the dosages and concentrations employed. The pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18^{th} Edition, (A. R. Gennaro, ed.), 1990, Mack Publishing Company.

Those of skill in the art will recognize that with respect to the compounds discussed herein, such compounds may contain a center of chirality. Thus, such agents may exist as different enantiomers or as enantiomeric mixtures. Use of any one enantiomer alone or contained within an enantiomeric mixture with one or more stereoisomers is contemplated by the present invention.

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefor. Pharmaceutical compositions of the invention can be prepared for storage by mixing the selected composition having the desired degree of purity with optional formulation agents (REMINGTON'S PHARMACEUTICAL SCIENCES, Id.) in the form of a lyophilized cake or an aqueous solution. Further, the compositions can be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The pharmaceutical compositions of the invention can be delivered parenterally.

When parenteral administration is contemplated, the therapeutic compositions for use in this invention may be in the form of a pyrogen-free, parenterally acceptable aqueous solution comprising the desired compound identified in a screening method of the invention in a pharmaceutically acceptable vehicle. A particularly suitable vehicle for parenteral injection is sterile distilled water in which the compound identified in a screening method of the invention is formulated as a sterile, isotonic solution, appropriately preserved. Preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which may then be delivered via a depot injection. Formulation with hyaluronic acid has the effect of promoting sustained duration in the circulation. Implantable drug delivery devices may be used to introduce the desired molecule.

The compositions may be formulated for inhalation. In these embodiments, a composition as disclosed herein can be formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery and is incorporated by reference.

The pharmaceutical compositions of the invention can be delivered through the digestive tract, such as orally. The preparation of such pharmaceutically acceptable compositions is within the skill of the art. A composition as disclosed herein that is to be administered in this fashion can be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the antagonist or agonist as disclosed herein. Diluents, flavorings, low melting point waxes, vegetable oils, lubricants, suspending agents, tablet disintegrating agents, and binders may also be employed.

A pharmaceutical composition can involve an effective quantity of a compound as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are evident to those skilled in the art, including formulations involving a compound as disclosed herein in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT Application No. PCT/US93/00829, which describes the controlled release of porous polymeric microparticles for the delivery of pharmaceutical compositions. Sustained-release preparations may include semipermeable polymer matrices in the form of shaped articles, e.g. films, or microcapsules, polyesters, hydrogels, polylactides (e.g., U.S. Pat. No. 3,773,919 and European Patent No. 058,481), copolymers of L-glutamic acid and gamma ethyl-L-glutamate (Sidman et al., 1983, Biopolymers, vol. 22, pp. 547-556), poly (2-hydroxyethyl-methacrylate) (Langer et al., 1981, J. Biomed. Mater. Res., vol. 15, pp. 167-277) and Langer, 1982, Chem. Tech., vol. 12, pp. 98-105), ethylene vinyl acetate (Langer et al., id.) or poly-D(−)-3-hydroxybutyric acid (European Patent No. 133,988). Sustained release compositions may also include liposomes, which can be prepared by any of several methods known in the art. See e.g. Eppstein et al., 1985, Proc. Natl. Acad. Sci. USA, vol. 82, pp. 3688-3692; European Patent No. 036,676; European Patent No. 088,046, and European Patent No. 143,949.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The present invention can include kits for producing a single-dose administration unit. Kits according to the invention can each contain both a first container having a dried antagonist or agonist compound as disclosed herein and a second container having an aqueous formulation, including for example single and multi-chambered pre-filled syringes (e.g., liquid syringes, lyosyringes or needle-free syringes).

The effective amount of a pharmaceutical composition of the invention to be employed therapeutically will depend, for example, upon the therapeutic context and objectives. One skilled in the art will appreciate that the appropriate dosage levels for treatment, according to certain embodiments, will thus vary depending, in part, upon the antagonist or agonist delivered, the indication for which the pharmaceutical composition is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 100 mg/kg; or 1 μg/kg up to about 100 mg/kg; or 5 μg/kg up to about 100 mg/kg.

The dosing frequency will depend upon the pharmacokinetic parameters of an antagonist or agonist as disclosed herein in the formulation. For example, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

Administration routes for the pharmaceutical compositions of the invention include orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

Pharmaceutical compositions of the invention can be administered alone or in combination with other therapeutic agents.

### EXAMPLE 1

### ADDL Localization and Targeting

### Materials and Methods

ADDLs and Fractionation: Aβ₁₋₄₂ peptide (California Peptide Research, Napa, Calif.) or biotin-Aβ₁₋₄₂ peptide (Recombinant Peptide, Athens, Ga.) were used to prepare synthetic ADDLs or biotinylated ADDLs according to published protocols (see e.g., Lambert, M. P. et al. (2001) J. Neurochem., vol. 79, pp. 595-605; Klein, W. L. (2002) Neurochem. Int., vol. 41, pp. 345-352; references in either of the foregoing; and the like). Molecular weight fractionation of oligomeric species was obtained using Centricon YM-100 and YM-10 concentrators (Millipore, Bedford, Mass.), used according to manufacturer's instructions. Size exclusion chromatography using the Akta Explorer HPLC apparatus with a Superdex 75 HR 10/30 column was conducted according to published protocols (see e.g., Chromy, B. A. et al. (2003) Biochemistry, vol. 42, pp. 12749-12760; references therein; and the like).

Tissue extracts and CSF: Frontal cortex, cerebellum and CSF from Alzheimer's disease and non-demented control subjects were obtained from the Northwestern Alzheimer's Disease Center Neuropathology Core (Chicago, Ill.). Soluble extracts from brain tissues were prepared as described previously (see e.g. Gong, Y. et al. (2003) Proc. Natl. Acad. Sci. USA, vol. 100, pp. 10417-10422; references therein; and the like).

Cell Culture: Hippocampal neurons were maintained in Neurobasal medium supplemented with B27 (Invitrogen, Carlsbad, Calif.) for at least three weeks as described previously (see e.g., Gong, Y. et al. (2003) Proc. Natl. Acad. Sci. USA, vol. 100, pp. 10417-10422; references therein; and the like). Cells were incubated with vehicle, synthetic or biotinylated ADDLs (500 nM), crude human CSF (100 μl) or F12-extracted human cortex (1.0 mg protein/ml) for indicated times.

Immunocytochemistry: Immunocytochemistry was performed as described (see e.g., Gong, Y. et al. (2003) Proc. Natl. Acad. Sci. USA, vol. 100, pp. 10417-10422; references therein; and the like). Some cells were permeabilized with 0.1% Triton in 10% normal goat serum and phosphate buffered saline (NGS:PBS) for 1 h at room temperature (RT). Cells were double-immunolabeled with M94 (Aβ oligomer-generated polyclonal antibody characterized earlier (see e.g., Lambert, M. P. et al. (2001) J. Neurochem., vol. 79, pp. 595-605; Klein, W. L. (2002) Neurochem. Int., vol. 41, pp. 345-352; references in either of the foregoing; and the like) (1:500) and either anti-αCaMKII (1:250) or anti-PSD-95 (1:500) monoclonal antibodies (Affinity BioReagents, Golden, Colo.) or goat polyclonal anti-Arc antibody (1:200) (Santa Cruz Biotechnology, Santa Cruz, Calif.), NMDA-R1 (C-term, 1:200) (Upstate, Lake placid, NY), or synaptophysin (SVP-38, 1:500) (Sigma, Saint Louis, Mo.) overnight at 4° C., followed by an incubation with appropriate AlexaFluor®488 or 594 conjugated IgG (Molecular Probes, Eugene, Oreg.) (2 μg/ml) for 2 h at RT. Double-labeling for ADDLs and either NMDA-glutamate receptor subunit NR1 (1:200) or AMPA-glutamate receptor subunit GluR1 (1:200) (Upstate Biotechnology, Lake Placid, N.Y.) used synthetic biotinylated ADDLs and streptavidin-AlexaFluor®488 conjugate. Cells were visualized using a Leica TCS SP2 Confocal Scanner DMRXE7 Microscope (Bannockburn, Ill.) with constant settings of laser power, detector gain, amplification gain, and offset. Images were acquired in z-series scans at 0.5 μm intervals from individual fields to determine whether ADDLs colocalized with αCaMKII, PSD-95, SVP-38, NR1, GluR1, or Arc. Morphometric quantifications were performed with MetaMorph imaging software (Universal Imaging Corp, West Chester, Pa.).

Immunohistochemistry: Autopsied brain from 7 AD cases (59 to 87 years old) and 7 non-demented elderly controls (68 to 78 years old) were immersion-fixed in 10% buffered formalin for 30-48 h then transferred to a 10-40% sucrose gradient. Free-floating 40 μm thick serial sections were obtained from frontal cortex and kept in cryoprotectant at 4° C. until immunolabeling. Sections were rinsed with TBS, pre-treated with 2% sodium m-periodate in TBS for 20 min and permeabilized with 0.25% Triton X-100 in TBS (TBST). Aspecific immunoreactivity was blocked with 5% horse serum in TBST for 40 min and 1% non-fat dry milk in TBST for 30 min. Sections were subsequently incubated with M94 (1:1000) overnight at 4° C. and AlexaFluor488 anti-rabbit IgG (1:500) for 90 min at RT. Serial sections were stained with 0.5% thioflavine-S in 50% ethanol. Confocal images were collected on the Leica confocal microscope as described above with z-series scans of 1 μm intervals. Similar sections were labeled with M94 antibody and secondary antibody/ABC complex and diaminobenzidine (DAB). Sections were counterstained with hematoxylin. Control sections with the primary or secondary antibodies omitted were negative.

Immunoblot: Cells were lysed in 1 volume PBS/protease inhibitor cocktail solution and 1 volume 2× loading buffer pH 6.8 (80 mM Tris-HCl, 16.7% glycerol, 1.67% SDS, 1.67% β-mercaptoethanol) and sonicated briefly. Proteins were separated on 4-20% Tris-glycine gels (BioRad, Hercules, Calif.) at 100V and transferred to nitrocellulose membrane at 100V for 1 h at 4° C. in transfer buffer (25 mM Tris-HCl, pH 8.3, 192 mM glycine, 20% v/v methanol). Blots were blocked with 5% non-fat dry milk in 10 mM Tris-buffered saline containing 0.1% Tween 20 pH7.5 for 2 h. Blots were incubated overnight at 4° C. with anti-Arc antibody (1:250) and 2 h with HRP-conjugated IgG (1:100,000). Membranes were developed with SuperSignal West Femto chemiluminescence kit (Pierce Biotechnology, Rockford, Ill.), then washed, blocked and reblotted with anti-cyclophilin B antibody (1:40,000) used as a control for protein loading. Proteins were visualized and quantified using the Kodak IS440CF Image Station (New Haven, Conn.).

Dot blot assay: A previously described dot blot assay was used to measure assembled forms of Aβ in soluble extracts of human frontal cortex and cerebellum (see e.g., Gong, Y. et al. (2003) Proc. Natl. Acad. Sci. USA, vol. 100, pp. 10417-10422; references therein; and the like). Nine AD samples (pathology diagnosis based on Braak & Braak, CERAD and NIA/Reagan Institute Criteria) were compared to fifteen non-AD control samples. Tissue (100 mg) was homogenized in 1 ml Ham's F12 phenol-free medium (BioSource, Camarillo, Calif.) containing protease inhibitors (Complete mini EDTA free tablet; Roche, Indianapolis, Ind.) on ice using a Tissue Tearor (Biospec Products, Bartlesville, Okla.). After centrifugation at 20,000 g for 10 min, the supernatant was centrifuged at 100,000 g for 60 min. Protein concentration of 100.000 g supernatant was determined by standard BCA assay. For dot blot assay, nitrocellulose was pre-wetted with TBS (20 mM Tris-HCl, pH 7.6, 137 mM NaCl) and partially dried. Extracts (2 μl, 1 μg total protein) were applied to nitrocellulose and air dried completely. The nitrocellulose membranes were then blocked in 0.1% Tween 20 in TBS (TBS-T) with 5% non-fat dry milk powder for 1 h at RT. The membranes were incubated for 1 h with primary antibody M93/3 in the blocking buffer (1:1000) and washed 3×15 min. with TBS-T. Incubation with HRP-conjugated secondary antibody (1:50,000, Amersham, Piscataway, N.J.) in TBS-T for 1 h at RT was followed by washes. Proteins were visualized with chemiluminescence and analyzed on a Kodak IS440CF Imaging Station with Kodak 1D image software.

### Results

### Soluble Aβ Species, Detected by Aβ Oligomer-Raised Antibody, are Deposited Around Neuronal Cell Bodies and Increased in AD Cortex:

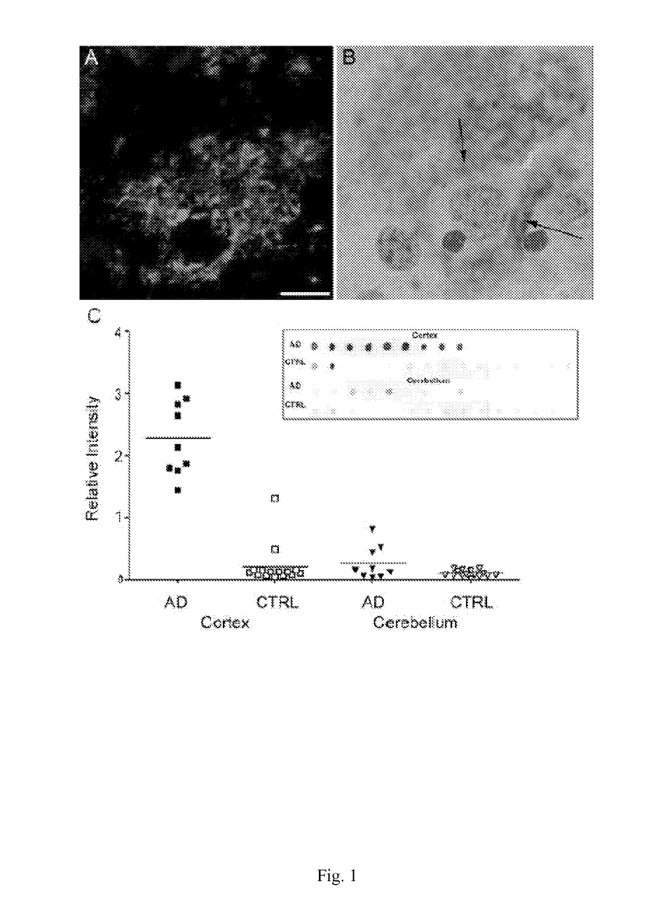

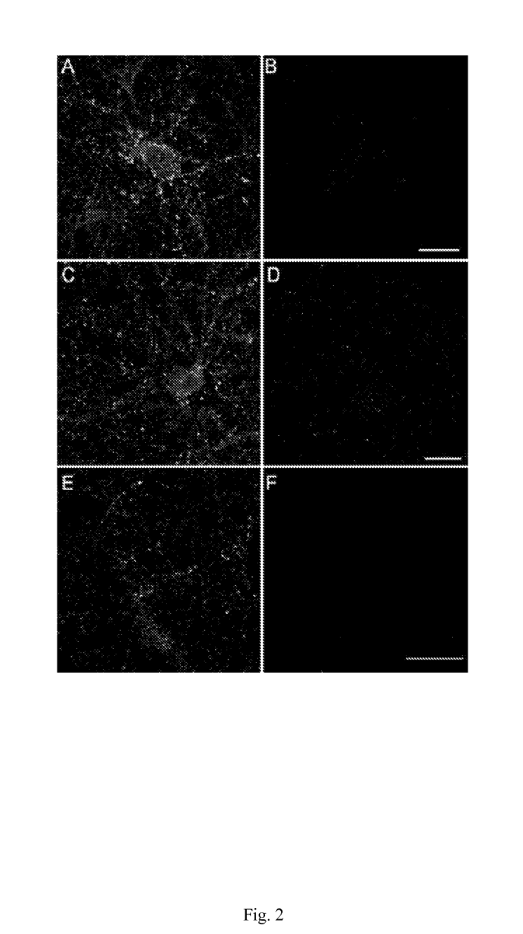

The first goal was to verify the presence of ADDLs in AD brain and to establish that the antibodies employed in subsequent cell biology experiments were specific for Alzheimer's pathology. Accordingly, sections from human frontal cortex (7 AD patients and non-demented age-matched controls) were immunolabeled with M94 (an oligomer-selective antibody (see e.g., Gong, Y. et al. (2003) Proc. Natl. Acad. Sci. USA, vol. 100, pp. 10417-10422; references therein; and the like); and assessed for fibrillar amyloid deposits with thioflavin-S. Immunolabeled AD brain sections exhibited localized immunoreactive deposits that selectively surrounded cell bodies in regions that also showed characteristic Aβ deposition in the forms of senile neuritic and diffuse amyloid plaques. However the pericellular diffuse immunoreactivity, which was found in all AD cases, was clearly distinct from fibrillar amyloid deposits (detected by thioflavin-S staining; not shown). Representative images of individual pyramidal neurons located in cortical layer III are shown labeled by immunofluorescence (FIG. 1A) and by HRP-staining (FIG. 1B). One control (of seven) showed similar structures; this particular control brain was Braak stage 0 with low levels of plaques from an individual with mild cognitive impairment. No immunoreactivity was observed in non-demented age-matched control frontal cortex (not shown). Overall, the diffuse oligomer staining in AD sections was pericellular rather than intracellular, reminiscent of the description of the diffuse synaptic type deposit observed with prion-associated diseases (see e.g., Hainfellner, J. A. et al. (1997) Brain Pathol., vol. 7, pp. 547-553; Kovacs, G. G. et al. (2002) Brain Pathol., vol. 12, pp. 1-11; references in either of the foregoing; and the like).

Dot blot immunoassays were used to verify the presence of oligomeric Aβ in soluble extracts of human frontal cortex and cerebellum (FIG. 1C, dot blot), with and without diagnosis of AD. Immunoreactivity was robust in all AD frontal cortex extracts. In controls, immunoreactivity was close to assay background for all cortex and cerebellum samples, with the exception of two cortical samples, from subject with low levels of plaques and tangles (plaque severity 1, CERAD A, Braak II, NIA/Reagan low). The mean signal in AD cortical samples is elevated ˜11-fold (p<0.0001), although the closeness of many control samples to assay background makes the magnitude of this ratio imprecise. In contrast to the cortical samples, levels of soluble oligomers in AD cerebellum were minimally greater, but not significantly, than in non-demented controls (p=0.1316) (FIG. 1C, scatter plot).

These results confirm and extend the report that soluble oligomers are bona fide constituents of AD pathology (see e.g. Gong, Y. et al. (2003) Proc. Natl. Acad. Sci. USA, vol. 100, pp. 10417-10422; references therein; and the like). They verify, furthermore, that the antibodies used previously to characterize soluble oligomers from AD brain specifically recognize AD brain pathology. The data thus validate use of these antibodies and soluble AD brain extracts in cell biological experiments, described below, that are designed to characterize the nature of interactions between ADDLs and neurons.

Aβ Oligomers (ADDLS) Extracted from AD Brain Bind Specifically to Clustered Sites:

ADDLs are small, diffusible oligomers of Aβ 1-42, an amphipathic peptide. Given their relatively favorable aqueous solubility compared to Aβ 1-42 monomers, it is likely that oligomers sequester their hydrophobic domains while presenting their hydrophilic domains to the aqueous environment. Such orientation is consistent with the immuno-neutralization of ADDLs in solution by conformation-sensitive antibodies (see e.g., Lambert, M. P. et al. (2001) J. Neurochem., vol. 79, pp. 595-605; references therein; and the like). Thus, ADDL structure is theoretically competent to accommodate a ligand-like specificity for memory-related neurons, in contrast to relatively non-specific binding associated with the reported insertion of Aβ monomer into artificial lipid bilayers (see e.g., McLaurin, J. & Chakrabartty, A. (1996) J. Biol. Chem., vol. 271, pp. 26482-26489; references therein; and the like).

To confirm this mode of high specificity ADDL binding, we investigated ADDL interactions with neurons under physiologically relevant conditions, using as our experimental model rat hippocampal neurons maintained in culture for at least three weeks. These cultures are synapse-generating (see e.g. Fong, D. K. et al. (2002) J. Neurosci., vol. 22, pp. 2153-2164; references therein; and the like) and produce mature, highly-differentiated neurons with complex arbors.

The first binding experiments were done with extracts of human brain and with human CSF. Soluble extracts of AD cortex, previously shown to contain oligomers that are structurally equivalent to oligomers prepared in vitro (see e.g., Gong, Y. et al. (2003) Proc. Natl. Acad. Sci. USA, vol. 100, pp. 10417-10422; references therein; and the like), were incubated with cultured neurons. Unbound material was removed by washes, and cells were examined by immunofluorescence microscopy. ADDL distribution was determined using polyclonal antibodies (M94) generated by vaccination with synthetic Aβ oligomers. These antibodies bind to low doses of pathogenic Aβ oligomers but not physiological monomers (see e.g. Chang, L. et al. (2003) J. Mol. Neurosci., vol. 20, pp. 305-313; Gong, Y. et al. (2003) Proc. Natl. Acad. Sci. USA, vol. 100, pp. 10417-10422; Lambert, M. P. et al. (2001) J. Neurochem., vol. 79, pp. 595-605; references in any of the foregoing; and the like) and, as described above, are specific for AD brain tissue.

Incubation of extracts with well-differentiated cultures of rat hippocampal cells, even for times as short as 5 min, resulted in membrane-type labeling along cell bodies and neurites (FIG. 2A). Under identical conditions, no signal was generated by extracts from age-matched non-demented controls (FIG. 2B). There was no indication that antibodies recognized physiological molecules such as Aβ monomer or amyloid precursor protein. Although Aβ 1-42 has been reported to accumulate intracellularly in AD and in transgenic mouse models of AD (see e.g., Oddo, S. et al. (2003) Neuron, vol. 39, pp. 409-421; Gouras, G. K. et al. (2000) Am. J. Pathol., vol. 156, pp. 15-20; references in either of the foregoing; and the like), ADDL immunoreactivity on neurons was exclusively at cell surfaces, even after permeabilization. Distribution was distinctly punctate in nature. Centricon filter fractionation of AD extracts showed that binding activity resided with oligomers of mass between 10-100 kD, consistent with previous characterization (see e.g., Gong, Y. et al. (2003) Proc. Natl. Acad. Sci. USA, vol. 100, pp. 10417-10422; references therein; and the like) FIG. 2C,D). Unfractionated extracts of human CSF also exhibited binding activity that was AD-dependent (FIG. 2E,F). It is likely that intracellular Aβ observed in transgenic AD models occurs because of high Aβ overproduction and accumulation within the cellular secretory pathway.

Results show that human-derived ADDLs, from Alzheimer's brain and CSF, are capable of highly selective binding to neuronal cell surfaces characterized by punctate clusters of binding sites found in abundance within neuritic arbors.

ADDLs Generated from Synthetic Aβ1-42 Bind Specifically to Clustered Sites:

The binding characteristics of ADDLs generated in vitro were investigated. Such preparations constitute the standard for investigating the neurological impact of oligomers. Use of these defined preparations eliminates unknown factors in extracts and CSF that could contribute to binding and its consequences. In addition, as tools for widespread use and convenient comparisons between laboratories, synthetic ADDLs provide a much more accessible preparation than human brain extracts or CSF.

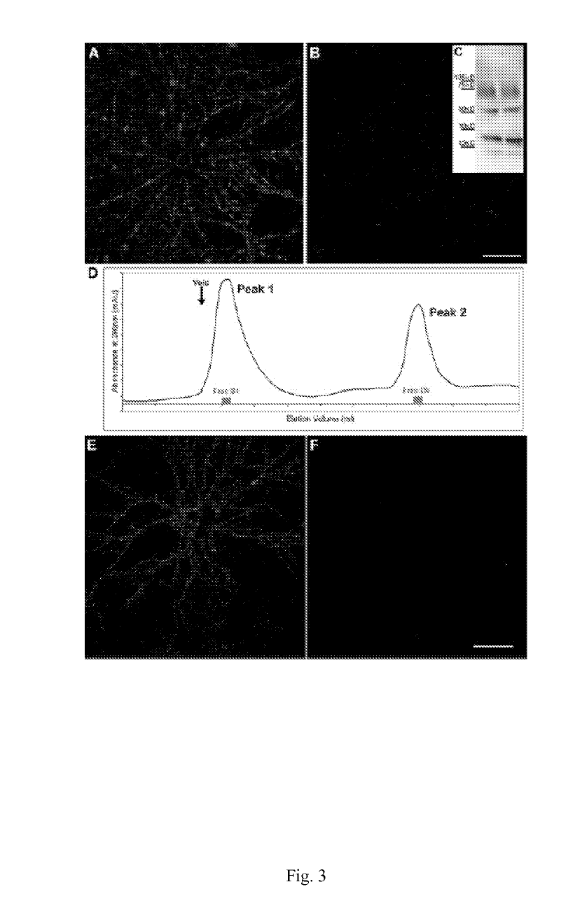

As observed with human preparations, ADDLs prepared in vitro and incubated with mature hippocampal neuronal cultures generated a specific binding pattern that exhibited abundant punctate sites within neuronal arbors. Pre-absorption of antibodies with synthetic oligomers produced no detectable signal (not shown), ruling out non-specific antibody association. Immunolabeling using an oligomer-specific monoclonal antibody (see e.g., Chromy, B. A. et al. (2003) Biochemistry, vol. 42, pp. 12749-12760; references therein; and the like) indicated the ligands were not monomers or fibrils (not shown), a conclusion substantiated by Centricon filter fractionation experiments. The 10-100 kDa Centricon fraction (FIG. 3A) but not the <10 kDa fraction (FIG. 3B) contained oligomers capable of binding to neuronal surfaces. As illustrated in FIG. 3C, there is no change in the Aβ species present in the cultured media during incubation (up to 6 hours) with hippocampal cells. The typical synthetic ADDL preparation contains SDS stable assemblies with molecular weights up to 24-mers, with a predominant 12-mer species, while AD brain extracts contain prevalent 12-mers (see e.g. Gong, Y. et al. (2003) Proc. Natl. Acad. Sci. USA, vol. 100, pp. 10417-1042248) and 48-mers (data not shown).

Additionally, neuronal binding of Aβ species was examined after separation by HPLC size-exclusion chromatography on Superdex 75 (as described in Chromy, B. A. et al. (2003) Biochemistry, vol. 42, pp. 12749-12760). Biotinylated ADDLs, prepared from biotin-Aβ₁₋₄₂ peptide, eluted in two peaks (FIG. 3D). Calibrated against known molecular weight standards, peak 1 contained species of an apparent molecular weight >50 kDa, consistent with 12-mers and larger species while peak 2 contained monomers and small oligomers. Dot blot, a method that detects all forms of assembled Aβ, was performed as in FIG. 1 on the eluted fractions to identify fractions with highest levels of immunoreactive material (not shown). These fractions were then tested for binding capacity on mature hippocampal cell cultures. The high molecular weight Aβ species contained in Peak 1 showed binding to hippocampal dendritic trees (FIG. 3E) while low molecular weight species from peak 2 did not bind (FIG. 3F).

Thus, results from fractionation by Centricon filters or chromatography, show the immunoreactivity imaged on neurons was not attributable to large molecules such as protofibrils nor to small molecules such as monomers or dimers.

### Selective Binding by Experimentally-Generated Oligomers—Clusters of Binding Sites and Cell-to-Cell Specificity:

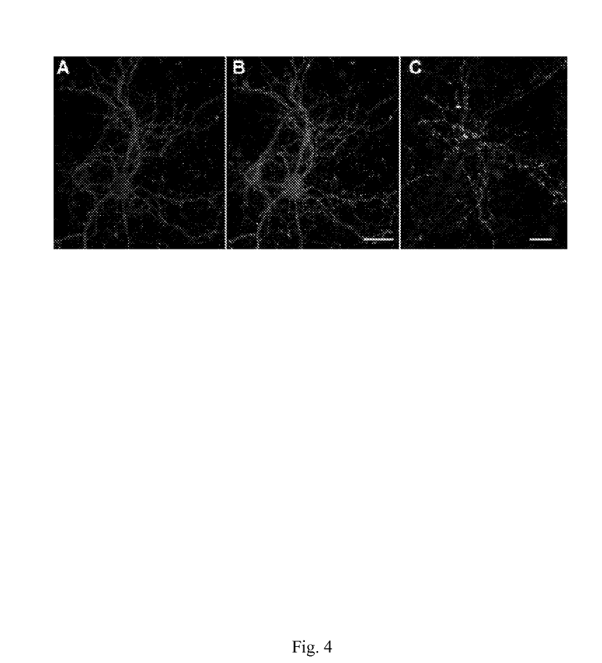

Contrary to what would be expected if ADDLs bound by non-selective membrane adsorption or insertion, all cells did not exhibit punctate clusters of binding sites. Cell-to-cell specificity in a double-label experiment is illustrated for a pair of αCaMKII-positive neurons (FIG. 4A,B), only one of which exhibits ADDL binding. Over many experiments, the subpopulation of cultured hippocampal neurons that bound ADDLs typically comprised 30-50% of the total in a given culture. These results establish the specificity of ADDL binding at the cellular level.

Clusters of ADDL Binding Sites are Coincident with Synapses:

At the subcellular level, whether ADDLs bind specifically to synapses is of great significance to the hypothesis that memory loss in AD is an oligomer-induced synaptic failure (see e.g., Lambert, M. P. et al. (1998) Proc. Natl. Acad. Sci. USA, vol. 95, pp. 6448-6453; Selkoe, D. J. (2002) Science, vol. 298, pp. 789-791; references in either of the foregoing; and the like). The rapidity with which ADDLs inhibit synaptic plasticity (see e.g., Lambert, M. P. et al. (1998) Proc. Natl. Acad. Sci. USA, vol. 95, pp. 6448-6453; Walsh, D. M. et al. (2002) Nature, vol. 416, pp. 535-539; references in either of the foregoing; and the like) suggests that the neurologically relevant binding might occur near synapses, while binding that specifically targets particular synapses would not only account for memory-specificity in AD but would also confer considerable constraints on the mechanism. Localization of ADDLs to punctate binding sites within dendritic arbors described above clearly is consistent with the hypothesis that ADDLs are synapse-specific ligands. However, the morphology of punctate binding sites also is consistent with other subcellular specializations such as membrane rafts or focal contacts.

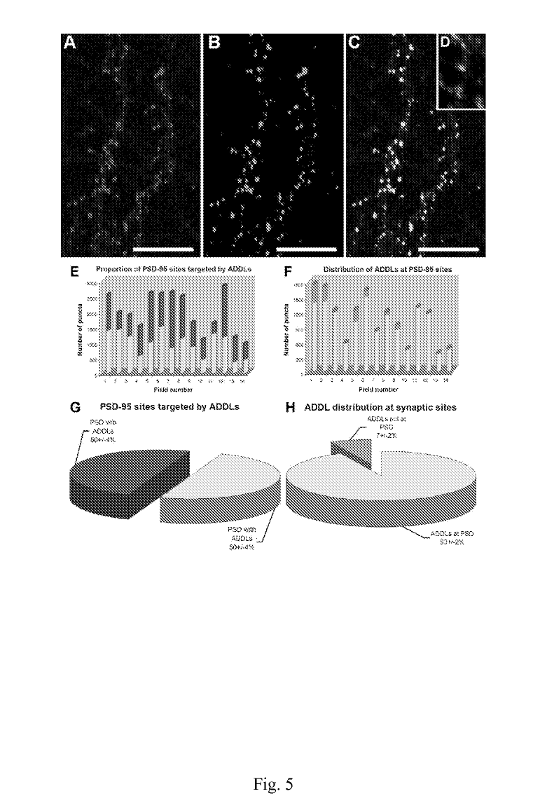

To test further the nature of oligomer binding site, the co-localization with PSD-95 was examined. PSD-95 is a critical scaffolding component of post-synaptic densities found in excitatory CNS signaling pathways (see e.g. Sheng, M. & Pak, D. T. (1999) Ann. N.Y. Acad. Sci., vol. 868, pp. 483-493; references therein; and the like), and clusters of PSD-95 previously have been established as definitive markers for post-synaptic terminals (see e.g. Rao, A. et al. (1998) J. Neurosci., vol. 18, pp. 1217-1229; references therein; and the like). In mature hippocampal cell cultures, such as used here, essentially all clusters of PSD-95 occur at synapses (see e.g., Allison, D. W. et al. (2000) J. Neurosci., vol. 20, pp. 4545-4554; references therein; and the like). As predicted, ADDL binding sites show striking coincidence with PSD-95 puncta, shown in an overlay at low magnification (FIG. 4C). Overlay analysis at higher magnification indicated the ADDL binding sites co-localized almost exclusively with puncta of PSD-95 (FIG. 5A-C). Identical patterns were obtained with extracts of AD brain (not shown). ADDL binding sites also were juxtaposed to synaptophysin-positive pre-synaptic terminals (FIG. 5D), although complete coincidence of ADDL and synaptophysin immunoreactivities was uncommon. To verify the apparent synaptic targeting by ADDLs, the extent of co-localization between ADDLs and PSD-95 was quantified by image analysis. Quantification of 14 fields showed that synthetic ADDLs colocalized with PSD-95 in 93+/−2% of the sites (FIG. 5F,H). ADDL binding sites thus were almost completely localized to synapses. These sites appeared to be selective, furthermore, for a synaptic subpopulation based on morphometric quantitation. At a certain threshold level for detecting ADDL puncta, approximately half of the PSD-95 puncta co-localized with ADDLs (FIG. 5E,G), although at a higher threshold for detecting ADDL clusters, it appears possible that all of the PSD-95 positive dendritic spines bound ADDLs.

ADDL binding sites, moreover, were found to overlap with NMDA receptor (NR1) immunoreactivity (not shown), consistent with the association of PSD-95 and NMDA glutamate receptors in excitatory hippocampal signaling pathways (see e.g., Sheng, M. & Pak, D. T. (1999) Ann. N.Y. Acad. Sci., vol. 868, pp. 483-493; references therein; and the like). ADDLs were also highly colocalized with PSD-family proteins and spinophilin (not shown). No overlap was evident between ADDLs and GluR1 (when using GluR1 C-terminal antibody which recognizes receptors expressed in dendritic shafts), phosphorylated tau (using Tau-1, an axonal marker), and SorLa (sorting protein-related receptor containing LDLR class A repeats, also called apolipoprotein E receptor LR11; a gift from Dr. H. C. Schaller). Co-localization thus was selective for synaptic markers.

The molecular basis for specific synaptic targeting by ADDLs is not known, although earlier studies with the B103 CNS neuronal cell line indicated specific binding to trypsin-sensitive cell surface proteins in flow cytometry experiments (see e.g. Lambert, M. P. et al. (1998) Proc. Natl. Acad. Sci. USA, vol. 95, pp. 6448-6453; references therein; and the like). A wide range of candidate proteins accumulate at PSD-95 synapses, including receptors for neurotransmitters, trophic factors, adhesion molecules and extracellular matrix proteins. In ligand overlay assays, which have the potential to detect binding to particular proteins that have been separated by SDS-PAGE, ADDLs bind with high affinity to two membrane-associated proteins from hippocampus and cortex (MW 140 and 260 kDa; (see e.g. Gong, Y. et al. (2003) Proc. Natl. Acad. Sci. USA, vol. 100, pp. 10417-10422; references therein; and the like). These proteins are also significantly enriched in isolated synaptosomes (800-900%; D. Khuon, personal communication). Identification of proteins corresponding to these two molecular weights was carried out by mass spectrometry. P140 corresponds predominantly to the post-synaptic protein synGAP, a 135 kDa protein known to stimulate ras GTPase activity. P260 corresponds predominantly to a post-synaptic scaffold protein known as proSAP2 or Shank3. synGAP is known to associates with PSD-95, while shank3 is known to associate with glutamate receptors.

### ADDLs Ectopically Up-Regulate the Synaptic Memory-Linked IEG Protein “Arc”:

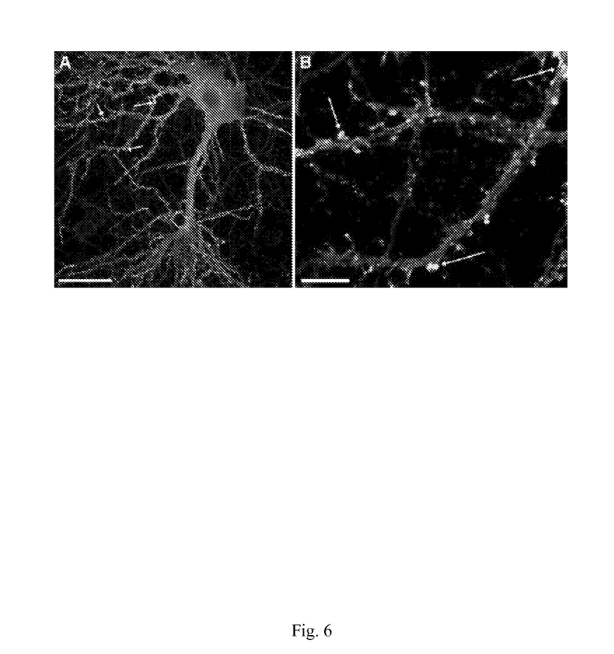

Synaptic relevance of the puncta is consistent with the striking specificity of ADDL binding to the highly arborized αCaMKII-positive neuron shown in FIG. 6A. This neuron shows discretely clustered sites found predominantly on dendrites; punctate binding is detectable on the cell body but at much lower density. At higher magnification (FIG. 6B), the composite overlays show numerous oligomer puncta capping the αCaMKII-positive dendritic spines. αCaMKII is known to accumulate in post-synaptic terminals of neurons linked to memory function, where it comprises over 30% of the protein in spiny post-synaptic terminals (see e.g., Inagaki, N. et al. (2000) J. Biol. Chem., vol. 275, pp. 27165-27171; references therein; and the like). The frequent localization of ADDL binding sites to dendritic spines suggests a potential for rapid impact on spine molecules.

To investigate this possibility further, the impact of ADDL binding on a synaptic immediate early gene mechanism critically linked to long-term memory formation was examined. The gene of interest codes for Arc (Activity-Regulated Cytoskeletal-associated) protein (see e.g. Guzowski, J. F. et al. (2000) J. Neurosci., vol. 20, pp. 3993-4001; references therein; and the like). Arc mRNA is targeted to synapses where, physiologically, the protein is induced transiently by synaptic activity (see e.g. Lyford, G. L. et al. (1995) Neuron, vol. 14, pp. 433-445; Link, W. et al. (1995) Proc. Natl. Acad. Sci. USA, vol. 92, pp. 5734-5738; Steward, O. & Worley, P. F. (2001) Proc. Natl. Acad. Sci. USA, vol. 98, pp. 7062-7068; references in any of the foregoing; and the like). Animal model studies have shown appropriate Arc expression is essential for LTP and for long-term memory formation (see e.g., Guzowski, J. F. et al. (2000) J. Neurosci., vol. 20, pp. 3993-4001; references therein; and the like). Besides being linked to drug abuse and sleep disruption (see e.g., Freeman, W. M. et al. (2002) Brain Res. Mol. Brain. Res., vol. 104, pp. 11-20; Cirelli, C. & Tononi, G. (2000) J. Neurosci., vol. 20, pp. 9187-9194; references in either of the foregoing; and the like), the ectopic and aberrant expression of Arc has been predicted to cause failure of long-term memory formation (see e.g. Guzowski, J. F. et al. (2000) J. Neurosci., vol. 20, pp. 3993-4001; references therein; and the like).

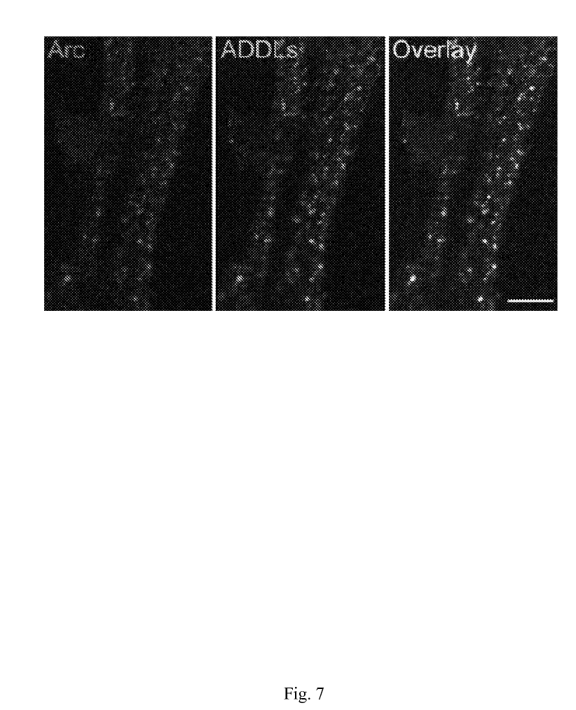

Hippocampal neurons were treated with ADDLs generated in vitro or vehicle for 5 min, 1 and 6 hours, and the impact on Arc protein determined by immunofluorescence and immunoblot. Synthetic ADDLs were used because, as discussed earlier, they are more readily obtained than AD-derived species and are uncontaminated by the myriad unknowns present in soluble brain extracts. At the earliest time point (5 min.), double-label immunofluorescence revealed that oligomer binding co-localized with dendritic punctate Arc expression (FIG. 7). This location appears to be an ectopic induction since low-levels of Arc protein that are expressed constitutively are known to localize in cell bodies, not at synapses (see e.g. Steward, O. & Worley, P. F. (2001) Proc. Natl. Acad. Sci. USA, vol. 98, pp. 7062-7068; references therein; and the like).

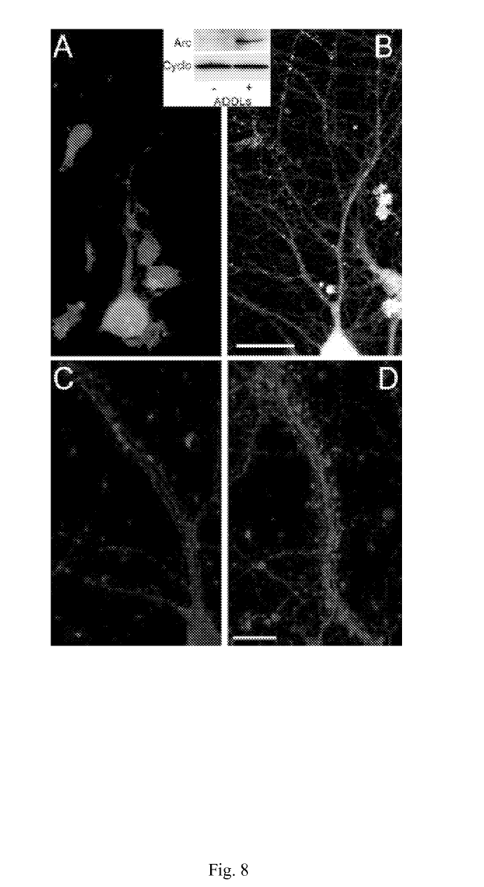

After longer exposure to ADDLs, the expression of Arc exhibited a robust upregulation. Expression of Arc throughout spines and dendrites was striking after 1 hour (FIG. 8B) and remained so after 6 hours (FIG. 8D) compared to vehicle-treated controls (FIGS. 8A and 8C). Elevated Arc expression also was evident in immunoblots (FIG. 8A-B insert), with low levels of Arc-IR in controls consistent with minimal basal Arc expression in neuronal cell bodies. The ADDL-induced increase in Arc was 5-fold over vehicle treated cultures.

Arc protein is coupled to F-actin and linked functionally to spine morphology, and its chronic over-expression has been suggested to generate abnormal spine structure (see e.g., Kelly, M. P. & Deadwyler, S. A. (2003) J. Neurosci., vol. 23, pp. 6443-6451; references therein; and the like). Examination of spine morphology in the current experiments indicated that Arc-positive spines in oligomer-treated groups differed from the few Arc-positive spines in control groups. Control spines expressing low level of Arc were stubby and laid close along dendritic shafts (FIG. 8C), whereas ADDL-treated spines were longer and appeared to extend from the dendritic shaft (FIG. 8D). Similar protruded spine structures were evident in treated cultures immunolabeled with anti-spinophilin (not shown).

### Discussion

ADDLs are neurologically harmful molecules that accumulate in AD brain (see e.g., Gong, Y. et al. (2003) Proc. Natl. Acad. Sci. USA, vol. 100, pp. 10417-10422; references therein; and the like). The current disclosure has addressed the cell biology of ADDL action, showing that ADDLs act as specific ligands for synaptic terminals, where they disrupt normal expression of a synaptic immediate early gene essential for long-term memory formation. The data provide a new molecular mechanism to support the emerging hypothesis that early AD memory loss results from ADDL-induced synapse failure, independent of neuron death and amyloid fibrils (see e.g. Hardy, J. & Selkoe, D. J. (2002) Science, vol. 297, pp. 353-356; Kirkitadze, M. D. et al. (2002) J. Neurosci. Res., vol. 69, pp. 567-577; Klein, W. L. et al. (2001) Trends Neurosci., vol. 24, pp. 219-224; Lambert, M. P. et al. (1998) Proc. Natl. Acad. Sci. USA, vol. 95, pp. 6448-6453; Selkoe, D. J. (2002) Science, vol. 298, pp. 789-791; references in any of the foregoing; and the like).

Previous clinical and mouse model data, coupled with studies of neurological impact in various experimental paradigms, strongly implicate non-fibrillar Aβ neurotoxins in AD memory loss (see e.g. Lambert, M. P. et al. (1998) Proc. Natl. Acad. Sci. USA, vol. 95, pp. 6448-6453; Mucke, L. et al. (2000) J. Neurosci., vol. 20, pp. 4050-4058; Walsh, D. M. et al. (2002) Nature, vol. 416, pp. 535-539; Walsh, D. M. & Selkoe, D. J. (2004) Protein Pept. Lett., vol. 11, pp. 213-228; Wang, H. W. et al. (2002) Brain Res., vol. 924, pp. 133-140; references in any of the foregoing; and the like). Roher and colleagues showed that soluble Aβ dimers are elevated in AD, although they initially were thought to be neurologically irrelevant, existing only as transient species en route to formation of toxic amyloid fibrils (see e.g. Lue, L. F. et al. (1999) Am. J. Pathol., vol. 155, pp. 853-862; references therein; and the like). Aβ oligomers now are established as stable molecular entities that exist for prolonged periods without conversion to fibrillar structures (see e.g. Chromy, B. A. et al. (2003) Biochemistry, vol. 42, pp. 12749-12760; references therein; and the like). Moreover, ADDLs are known to be potent CNS neurotoxins (see e.g. Lambert, M. P. et al. (1998) Proc. Natl. Acad. Sci. USA, vol. 95, pp. 6448-6453; references therein; and the like). Most relevant to early AD, ADDLs inhibit LTP. Observed ex vivo and in vivo, inhibition is rapid, non-degenerative and highly selective. The impact of ADDLS on synaptic plasticity likely accounts for plaque-independent cognitive failures seen in hAPP transgenic mice (see e.g. Hsia, A. Y. et al. (1999) Proc. Natl. Acad. Sci. USA, vol. 96, pp. 3228-3233; Mucke, L et al. (2000) J. Neurosci., vol. 20, pp. 4050-4058; Buttini, M. et al. (2002) J. Neurosci., vol. 22, pp. 10539-10548; Van Dam, D. et al. (2003) Eur. J. Neurosci., vol. 17, pp. 388-396; references in any of the foregoing; and the like), which accumulate ADDLs in an age-, region-, and transgene-dependent manner (see e.g., Chang, L. et al. (2003) J. Mol. Neurosci., vol. 20, pp. 305-313; references therein; and the like). It is likely that ADDLs are the targets of therapeutic antibodies that reverse memory loss in hAPP mice, a recovery that is both rapid and unrelated to plaque burden.

The presence of antigens detected by oligomer-specific antibodies also has been found in AD brain sections (see e.g., Kayed, R. et al. (2003) Science, vol. 300, pp. 486-489; references therein; and the like). Localization of these antigens is distinct from neuritic plaques, establishing the in situ presence of oligomers independent of fibrils. Patterns observed in the current investigation are consistent with this earlier report. A perineuronal distribution seen here, moreover, suggests localization of ADDLs to dendritic arbors. Dendritic binding by ADDLs extracted from AD brain tissue previously has been observed in experiments with cultured hippocampal neurons (see e.g. Gong, Y. et al. (2003) Proc. Natl. Acad. Sci. USA, vol. 100, pp. 10417-10422; references therein; and the like). Current results show the ligands in AD brain extracts are between 10 and 100 kDa, consistent with analysis of soluble AD brain extracts by 2D gel immunoblots, which established a predominant 12-mer (56 kDa) species. The 12-mers of AD brain are indistinguishable from 12-mers found in ADDL preparations generated in vitro with respect to isoelectric point, recognition by conformation-sensitive antibody, and ability to bind selectively to dendritic arbors.

New data presented here establish that the dendritic targets of ADDLs are synaptic terminals. Although this finding is in harmony with the hypothesis that ADDLs cause synapse failure, the size and distribution of the punctate binding sites might also be explained by binding to membrane rafts or focal contacts. In the current experiments, confocal immunofluorescence microscopy was used to compare localization of oligomers with a well-established synaptic marker, PSD-95. In mature hippocampal cultures, as used here, PSD-95 puncta are essentially 100% synaptic (see e.g. Rao, A. et al. (1998) J. Neurosci., vol. 18, pp. 1217-1229; references therein; and the like). ADDLs, whether generated in vitro or from AD brain, were found to co-localize almost exclusively with synapses. It is noteworthy that oligomers do not bind all neurons and synapses, but the particular phenotypes that are targeted remain to be elucidated. Preliminary experiments indicate, however, that the targeted synapses contain glutamate receptors. Another unanswered issue is the relationship between binding to synapses in culture and the diffuse stain shown by oligomers in AD brain sections. Data are consistent with the hypothesis that diffuse stain in situ would have a synaptic origin, currently under investigation by EM immunogold analysis.

If synapses were targeted in situ, the impact on memory ultimately would depend on the number of synapses targeted, the extent to which individual synapses are compromised, and the relevance of affected synapses to the overall process of memory formation. Synaptic impact could even be reversed spontaneously if ligands disassociated or their binding sites were turned over. Such complexities, although in harmony with the concept of synaptic reserve and day-to-day fluctuations in cognitive function, make it difficult to predict a simple relationship between oligomer levels and memory loss. It does seem likely, however, that a threshold of occupancy must be surpassed before memory loss would manifest.

The response of Arc to ADDLs is particularly intriguing because of Arc's putative involvement in long-term memory formation (see e.g., Guzowski, J. F. (2002) Hippocampus, vol. 12, pp. 86-104; references therein; and the like). Physiologically, Arc expression is controlled by patterned synaptic activity (see e.g., Lyford, G. L. et al. (1995) Neuron, vol. 14, pp. 433-445; Link, W. et al. (1995) Proc. Natl. Acad. Sci. USA, vol. 92, pp. 5734-5738; references in either of the foregoing; and the like) and occurs in recently activated dendritic spines (see e.g. Moga, D. E. et al. (2004) Neuroscience, vol. 125, pp. 7-11; references therein; and the like). In dendrites, Arc mRNA is localized to synaptic spines, consistent with co-localization of Arc and oligomers at early time points. It has been noted previously that Arc mRNA decreases with age in a tg-mouse AD model, although the relevance to synaptic Arc protein expression in this model was not determined (see e.g., Dickey, C. A. et al. (2003) J. Neurosci., vol. 23, pp. 5219-5226; references therein; and the like). In the current study, ADDLs caused sustained Arc induction leading to ectopic diffusion of protein throughout the dendritic arbor. Normally, Arc protein functions in a pulsate or transient manner, and it has been proposed that sustained Arc expression would generate synaptic noise and thereby inhibit long-term memory formation (see e.g., Guzowski, J. F. (2002) Hippocampus, vol. 12, pp. 86-104; references therein; and the like). This prediction is supported by findings from tg-mice that showed a correlation between elevated Arc and slow learning ability (see e.g. Kelly, M. P. & Deadwyler, S. A. (2003) J. Neurosci., vol. 23, pp. 6443-6451; references therein; and the like).

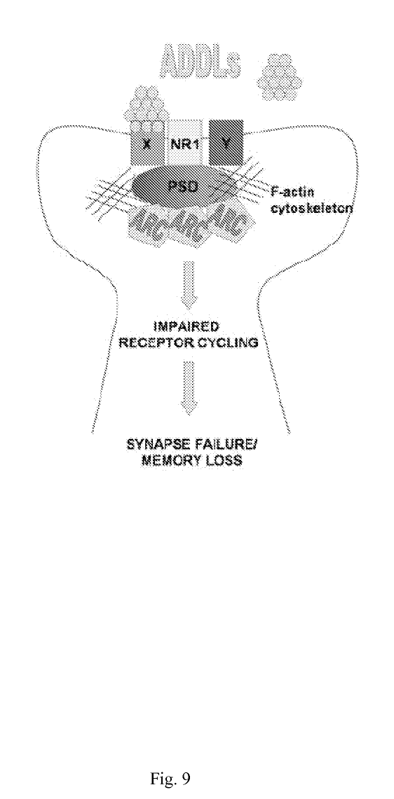

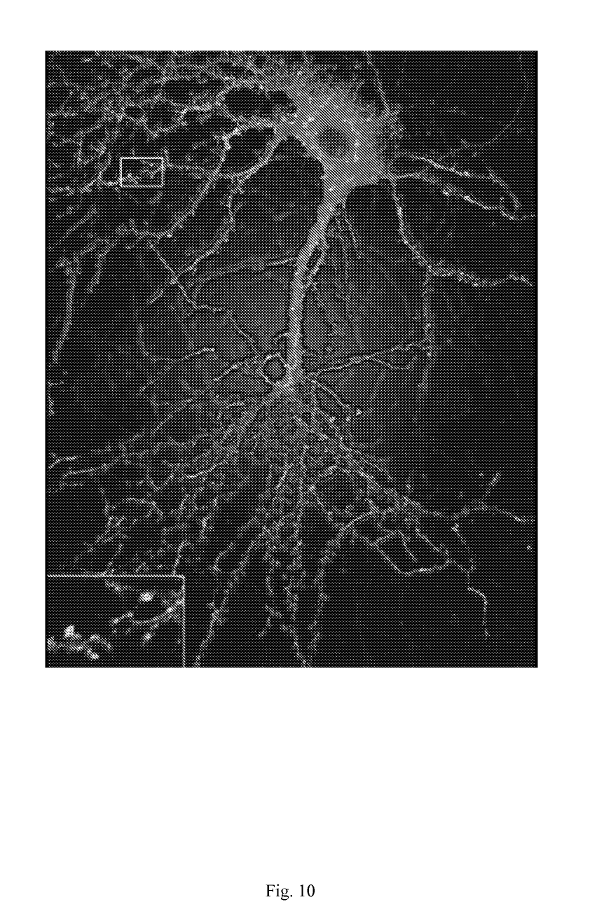

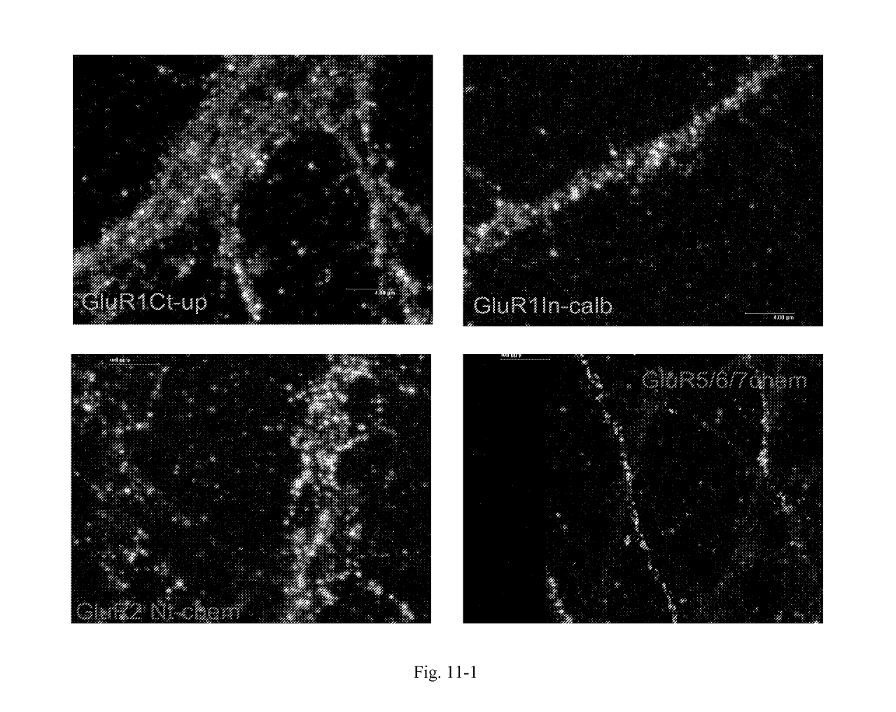

How the impact of ectopic Arc could lead to synapse failure may involve spine shape or receptor trafficking (FIG. 9). Arc is associated with cytoskeletal and post-synaptic proteins (see e.g., Lyford, G. L. et al. (1995) Neuron, vol. 14, pp. 433-445; Fujimoto, T. et al. (2004) J. Neurosci. Res., vol. 76, pp. 51-63; references in either of the foregoing; and the like), and it has been proposed that Arc elevation in tg-mice causes stiffening of synaptic spines, which would interfere with structural plasticity and retard learning (see e.g. Kelly, M. P. & Deadwyler, S. A. (2003) J. Neurosci., vol. 23, pp. 6443-6451; references therein; and the like). Synaptic spine abnormalities are common to various brain dysfunctions (see e.g., Fiala, J. C. et al. (2002) Brain Res. Brain Res. Rev., vol. 39, pp. 29-54; references therein; and the like) including mental retardation, where spines are atypically bent and protruding (see e.g. Kaufmann, W. E. & Moser, H. W. (2000) Cereb. Cortex., vol. 10, pp. 981-991; references therein; and the like). Spine abnormalities could rapidly alter synaptic signal processing and related information storage (see e.g. Crick, F. (1982) Trends Neurosci., vol. 5, pp. 44-46; Rao, A. & Craig, A. M. (2000) Hippocampus, vol. 10; pp. 527-541; Yuste, R. & Bonhoeffer, T. (2001) Annu. Rev. Neurosci., vol. 24, pp. 1071-1089; references therein; and the like). Elevated Arc also could disrupt cycling of receptors required for synaptic plasticity, e.g., blocking upregulation of AMPA receptors. Consistent with Arc cell biology, this disruption could derive from effects on cytoskeletal organization (e.g., f-actin or PSDs) or signaling pathways (e.g., via CaMKII) through a mechanism that may concomitantly alter spine structure.

Other synaptic signal transduction pathways also are affected by oligomers in culture models. In cortical cultures, low concentration of oligomers, at sublethal doses, inhibit the ability of glutamate to induce CREB phosphorylation (see e.g., Tong, L. et al. (2001) J. Biol. Chem., vol. 276, pp. 17301-17306; references therein; and the like), a signaling pathway associated with synaptic plasticity (see e.g., Sweatt, J. D. (2001) J. Neurochem., vol. 76, pp. 1-10; references therein; and the like). In hippocampal slice cultures, recent pharmacological studies indicate that LTP inhibition by oligomers involves particular kinases. Potentially of great interest, inhibitors of p38MAPK, JNK and cdk5 block the LTP impact of oligomers, as do antagonists of the type 5 metabotropic glutamate receptor (see e.g., Wang, Z, et al. (2004) J. Med. Chem., vol. 47, pp. 3329-3333; references therein; and the like). The inventors also suggest the putative involvement of receptors in the action of oligomers, and a number of candidate receptors have been hypothesized, no published data has established the identity of receptor proteins that mediate synaptic ADDL binding (see e.g. Verdier, Y. et al. (2004) J. Pept. Sci., vol. 10, pp. 229-248; references therein; and the like). Whether the signaling events described above and Arc induction are parallel or sequential with respect to the impact of ADDLs on Arc remains to be determined.

Action of ADDLs as disruptive synaptic ligands would provide an intuitively appealing mechanism for AD synapse failure. The key is that synapses themselves are targeted. There is no need to explain how memory-specific loss might derive from non-specific cellular associations (e.g., random insertion into cell membranes (see e.g., Gibson, W. W. et al. (2003) Biochim. Biophys. Acta, vol. 1610, pp. 281-290; references therein; and the like). The current data suggest a parsimonious mechanism in which memory-initiating events are locally disrupted at synapses. Ultimately, with prolonged exposure, the synapses targeted by oligomers may undergo physical degeneration mediated by related synaptic signaling molecules such as, e.g., Fyn (see e.g., Chin, J et al. (2004) J. Neurosci., vol. 24, pp. 4692-4697; references therein; and the like). ADDLs have been proposed to account for plaque-independent loss of terminals in some transgenic models of early AD (see e.g., Mucke, L. et al. (2000) J. Neurosci., vol. 20, pp. 4050-4058; references therein; and the like). Reports of age-dependent decreases in Arc mRNA in tg-mice are consistent with possible early stages of synaptic deterioration, although frank loss of terminals does not appear to occur in these strains (see e.g., Dickey, C. A. et al. (2003) J. Neurosci., vol. 23, pp. 5219-5226; references therein; and the like).

In end-stage AD, cognitive degeneration extends far beyond memory loss (see e.g., Coyle, J. T. (1987) Alzheimer's Disease. In: Encyclopedia of Neuroscience (Adelman G, ed), pp 29-31. Boston-Basel-Stuttgart: Birkhäuser; references therein; and the like). The hope would be that by blocking early pathogenic events, this downstream catastrophic cascade might never be reached. Results from human vaccine trials indicate therapeutic antibodies that target Aβ-derived neurotoxins might indeed halt disease progression (see e.g., Hock, C. et al. (2003) Neuron, vol. 38, pp. 547-554; references therein; and the like), although the incidence of significant brain inflammation with active vaccines complicates this strategy (see e.g., Schenk, D. (2002) Nat. Rev. Neurosci., vol. 3, pp. 824-828; Weiner, H. L. & Selkoe, D. J. (2002) Nature, vol. 420, pp. 879-884; references in either of the foregoing; and the like). Passive vaccination, while more expensive, would have fewer side-effects and, moreover, overcome the problem of compromised immune response common in the elderly. Monoclonal antibodies that immuno-neutralize soluble Aβ-species have been shown in two independent studies to reverse memory loss in tg-mice models of early AD (see e.g., Dodart, J. C. et al. (2002) Nat. Neurosci., vol. 5, pp. 452-457; Kotilinek, L. A. et al. (2002) J. Neurosci., vol. 22, pp. 6331-6335; references in either of the foregoing; and the like). It has been possible, moreover, to use ADDLs to generate antibodies that are specific for toxic forms of Aβ with minimal affinity for physiological monomers (see e.g., Lambert, M. P. et al. (2001) J. Neurochem., vol. 79, pp. 595-605; references therein; and the like). Recent hybridoma work, furthermore, indicates it is possible to generate antibodies that bind oligomers, but not amyloid fibrils, reducing concerns of inflammation caused by antibodies bound to plaques (see e.g., Chromy, B. A. et al. (2003) Biochemistry, vol. 42, pp. 12749-12760; references therein; and the like)(Chromy et al., 2003). The prospects for developing human therapeutic antibodies that target memory-relevant Aβ assemblies thus seem encouraging.

### EXAMPLE 2

### Receptor—ADDL Apposition and Co-Localization

Receptor—ADDL apposition and co-localization was performed essentially as described in Lacor, P. N. et al. (2004) J. Neurosci., vol. 24, no. 45, pp. 10191-10200. Briefly, hippocampal (HP) cells cultured for 3 weeks were treated with 500 nM ADDLs for 30 min, fixed, and washed 5 times. Immunolabeling was done with or without 0.1% Triton X-100 permeabilization depending on the antibody used (i.e., if an anti-Aβ N-terminal antibody was used, then non-permeabilized conditions were employed). Double labeling was done either with an anti-glutamate-receptor monoclonal antibody+the M71 anti-ADDL polyclonal antibody, or with a polyclonal glutamate-receptor antibody+the 20C2 anti-ADDL monoclonal antibody (see e.g., U.S. Patent App. No. 60/621,776; filed 25 Oct. 2004. Immunoreactivity was imaged using confocal microscopy.

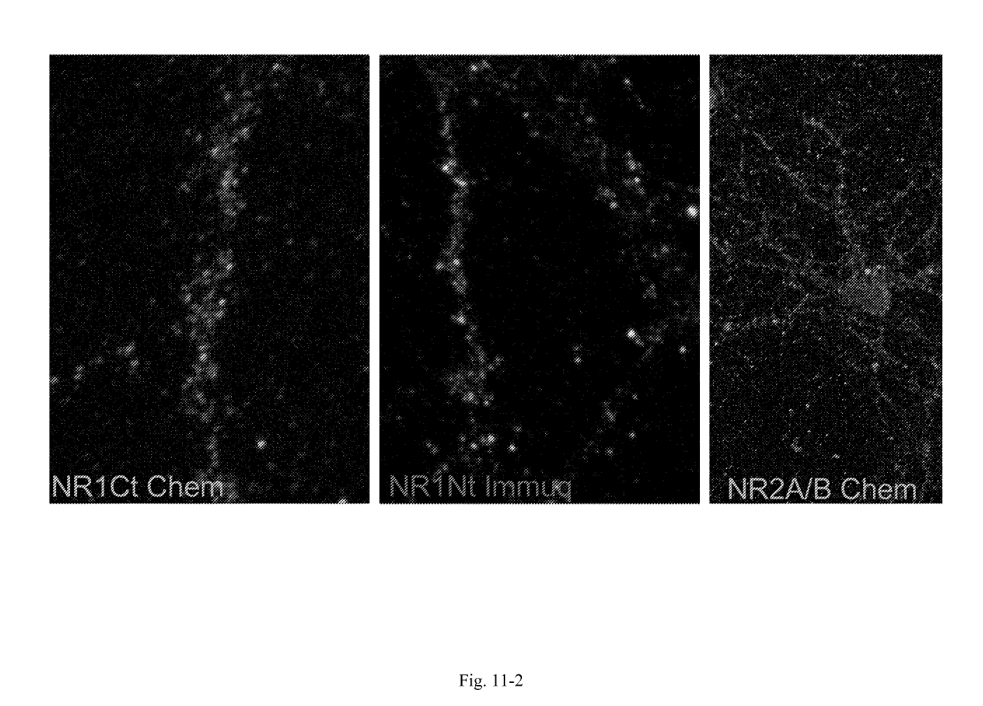

Referring to FIG. 11-1: The color of the glutamate (AMPA or KAINATE) receptor antibody as indicated in the panels matches the color of immunoreactivity (IR). ADDL-IR is in an opposing color. Colocalization of ADDLs and glutamate receptors is seen in yellow. Images represent a portion of the dendritic tree of 3 week old HP cells double-labeled for the indicated receptor and ADDLs. Images were acquired under 100× objective and digitally zoomed 3.5×. Scale bar represents 4 μm (micron). The data presented represents two different experiments with 6 fields per condition having been captured.

Referring to FIG. 11-2: The color of glutamate (NMDA) receptor antibody as indicated in the panels matches the color of immunoreactivity (IR). ADDL-IR is in an opposing color. Colocalization is seen in yellow. Images represent a portion of the dendritic tree of 3 week old HP cells double-labeled for the indicated receptor and ADDLs. Images were acquired under 100× objective and digitally zoomed 3.5×, except for the panel labeled ‘NR2A/B Chem’ which was not digitally zoomed. The data presented represents one experiment with 6 fields per condition having been captured.

Analysis: This information represents qualitative representations of colocalization between specific glutamate receptors and ADDLs. While not being bound by any one interpretation, preliminary results suggests that AMPA-R are more often colocalized with ADDLs than NMDA-R. ADDLs are always found on dendrites expressing glutamate receptors (AMPA-R or NMDA-R) and often juxtaposed to those receptors.

### EXAMPLE 3

### ADDL Impact on Receptors

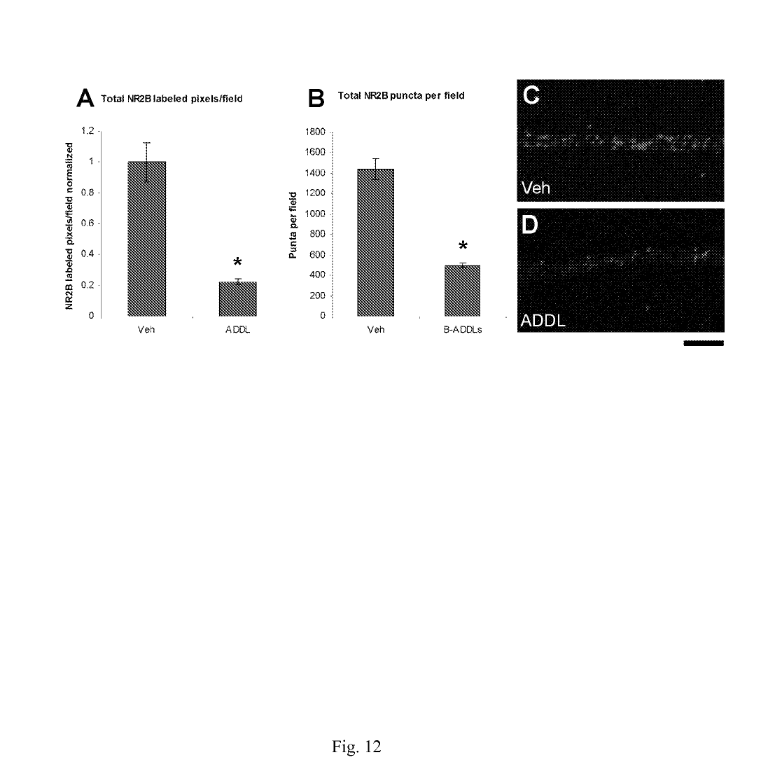

As shown in FIG. 12, NR2B membrane expression is decreased after ADDL exposure. Under non-permeablizing conditions, the amount of NR2B membrane expression was assessed using an antibody against an extracellular epitope. Equally dense neuropiles were imaged to allow for comparative analysis of NR2B labeling. A significant decrease in the total number of labeled pixels was observed which corresponded with a decrease in the number of NR2B puncta (p<0.001, n=4 neuropile images from one experiment, observed in two separate experiments). An example of NR2B labeling along a neurite is representative of the observed decrease in NR2B labeling after ADDL treatment in neuropiles imaged for quantification (C, D, scale bar represents 8 μm.)

### EXAMPLE 4

### ADDL Impact on Spine Geometry

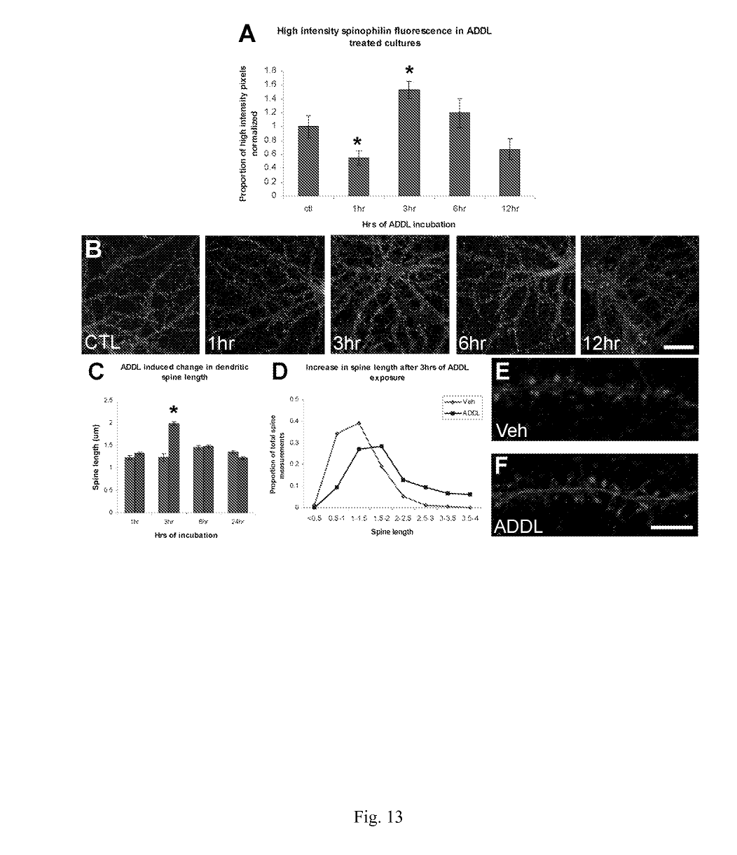

As shown in FIG. 13, Time-course treatment of hippocampal neurons with ADDLs results in a temporal post-synaptic response monitored by spinophilin immunofluorescence (IF) intensity and spine morphology. Time-course treatment of hippocampal neurons with ADDLs reveal a decrease in spinophilin fluorescence after 1 hr, which peaks significantly at 3 hrs before returning to control levels (A, p<0.05, data graphed is an average of 5 neurons imaged from one experiment and the corresponding SEM). Representative images of spinophilin IF after ADDL exposure are shown. (B, scale bar respresents 30 μm). Spine length was also measured after time-course treatment with ADDLs and a significant increase in spine length was observed after 3 hrs of ADDL incubation (C, p<0.005, data graphed is an average of spine length obtained from 10 dendritic branches from different neurons imaged in one experiment). The distribution of spine length measurements demonstrates an ADDL induced shift towards longer spines (D). High magnification images of spinophilin IF were used for spine length quantification (E,F, representative images after 3 hr of ADDL/vehicle incubation, scale bar represents 8 μm.)

### EXAMPLE 5

### ADDL Impact on Erb-B4 Receptors

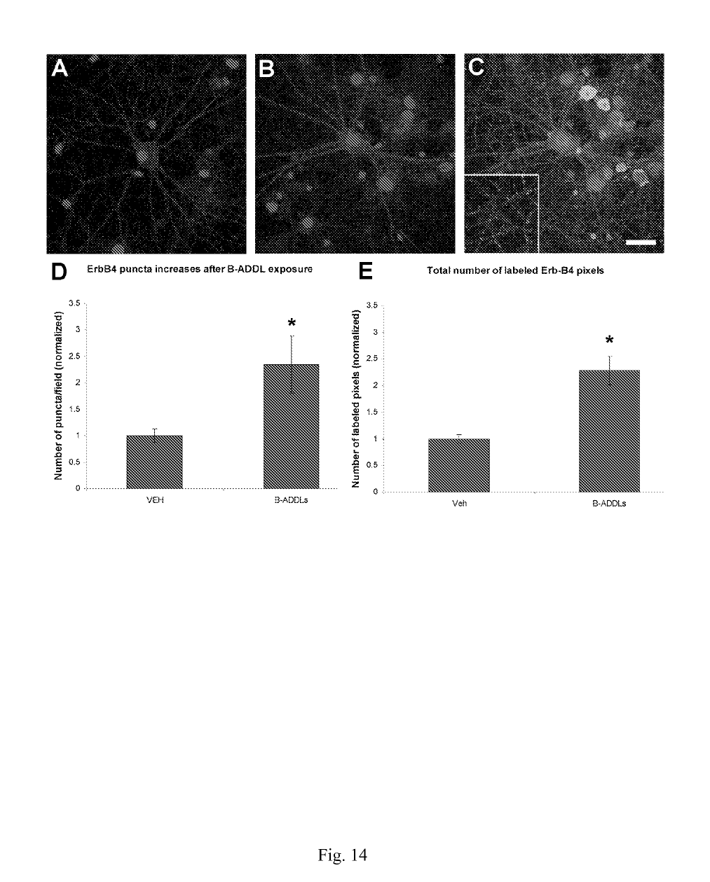

As shown in FIG. 14, Erb-B4 IF staining intensity is increased after 1 hr of ADDL exposure. Mature hippocampal neurons were treated with vehicle (A) and ADDLs (B) then immunolabeled for Erb-B4. Erb-B4 (red) is expressed strongly in a select number of cells which are not targeted by ADDLs, demonstrated by the image merging ErbB4 and ADDL (green) immunoreactivity (C). The inset is a higher magnification image of an ADDL bound neuropile showing the lack of co-localization between Erb-B4 and ADDL puncta. Quantification of ErbB4 IF in equally dense neuropiles revealed a significant increase in the number of labeled pixels and puncta (D, E, p<0.05, graphs show averages of 4 images obtained from one experiment and the corresponding SEM). Scale bar represents 40 μm.

### EXAMPLE 6

### ADDL Binding to Post-Synaptic Density (PSD)

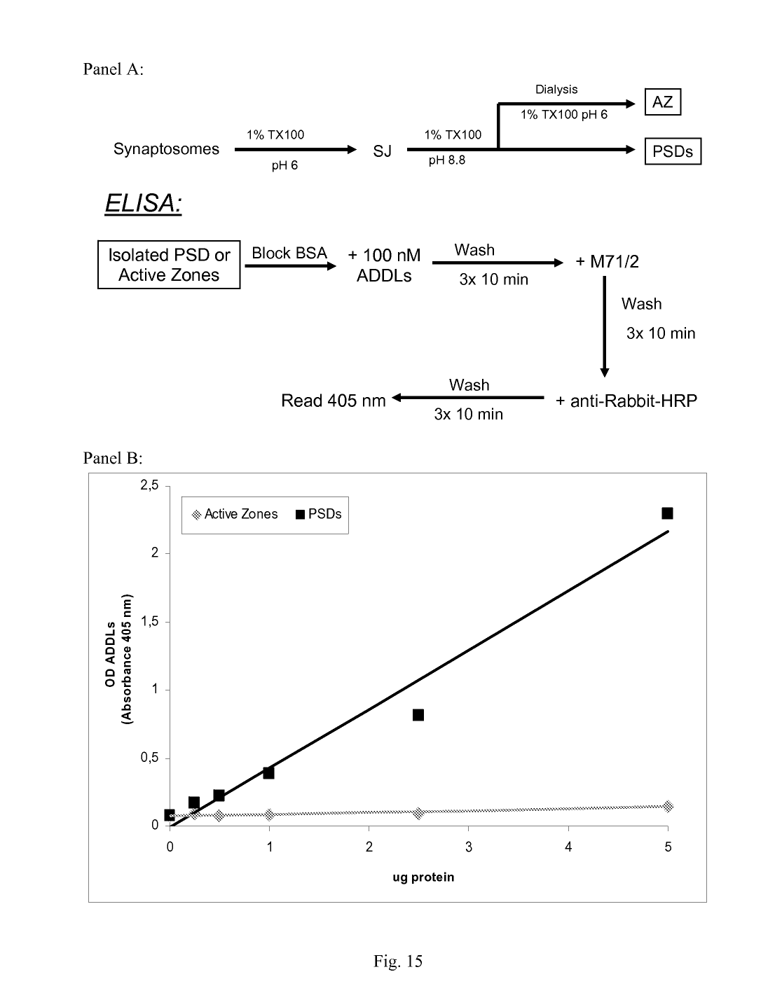

As shown in FIG. 15, ADDLs bind to post-synaptic densities (PSDs) and not active zones (AZs), as determined with an ELISA assay. The binding of ADDLs to PSDs was assayed by incubating isolated PSDs attached to a ELISA plate with ADDLs. Active zones (AZs) were used as a control. Panel A in FIG. 15 outlines a typical protocol for assaying ADDL binding to PSDs. As shown in the top part of Panel A, initially, synaptosomes are used to generate PSDs and AZs according to standard protocols (see e.g., Phillips, G. R. et al. (2001), Neuron, vol. 32, pp. 63-77; references therein, and the like). In the Figure, as well as elsewhere herein, TX100 represents Triton X-100. M71/2 designates an ADDL-specific poly clonal antibody, similar to M93 and M94 disclosed previously (see e.g. U.S. patent application Ser. No. 10/166,856; filed 11 Jun. 2002). Panel B in FIG. 15 represents typical results from such an assay.

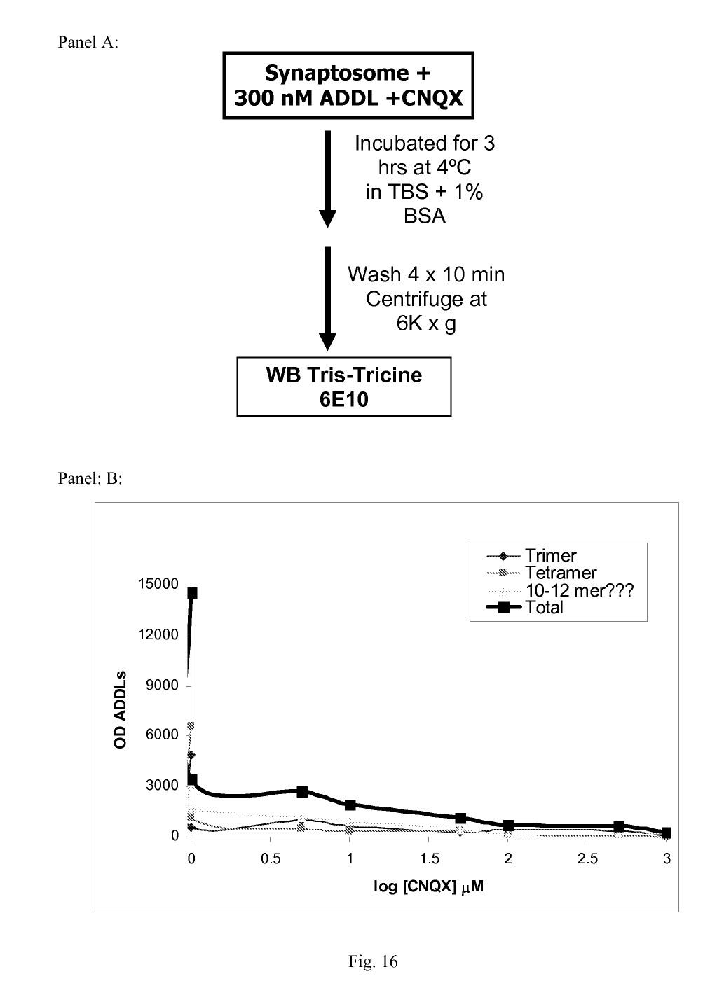

As shown in FIG. 16, CNQX blocks ADDL binding to synaptosomes. Panel A in FIG. 16 outlines a typical protocol for assaying ADDL binding to synaptosomes in the presence of CNQX. Panel B in FIG. 16 represents typical results from such an assay. WB stands for Western Blot, in this case using the 6E10 antibody.

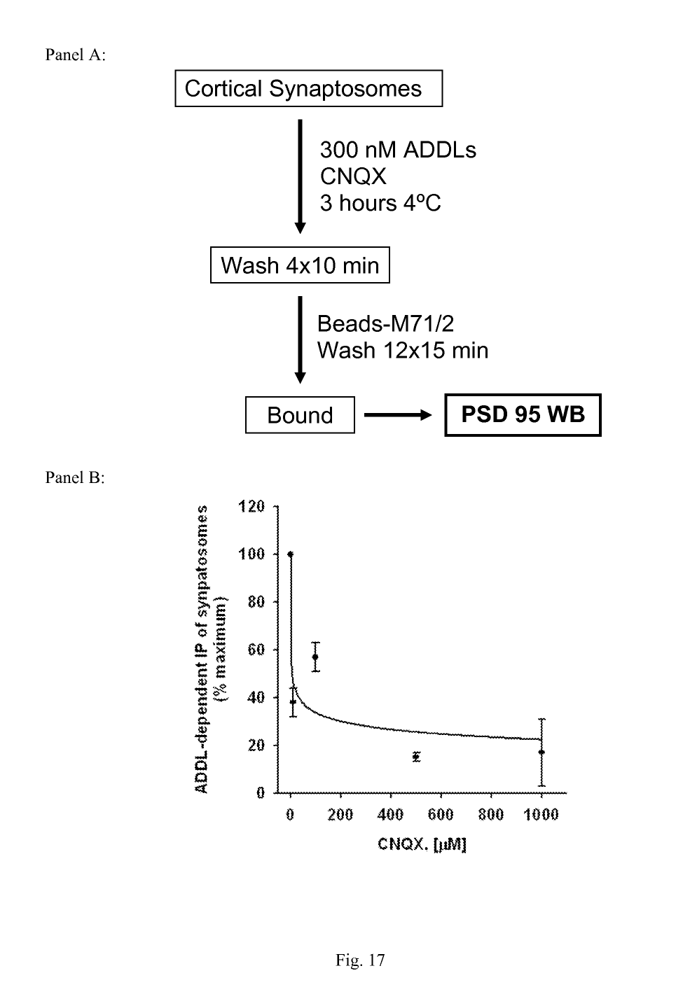

As shown in FIG. 17, CNQX decreases the amount of PSD-95 co-precipitated in an ADDL immuno-precipitation assay. Panel A in FIG. 17 outlines a typical protocol for assaying ADDL binding to PSD-95 in the presence of CNQX. Panel B in FIG. 17 represents typical results for such an assay. PSD-95 WB stands for PSD-95 Western Blot carried out according to standard protocols.

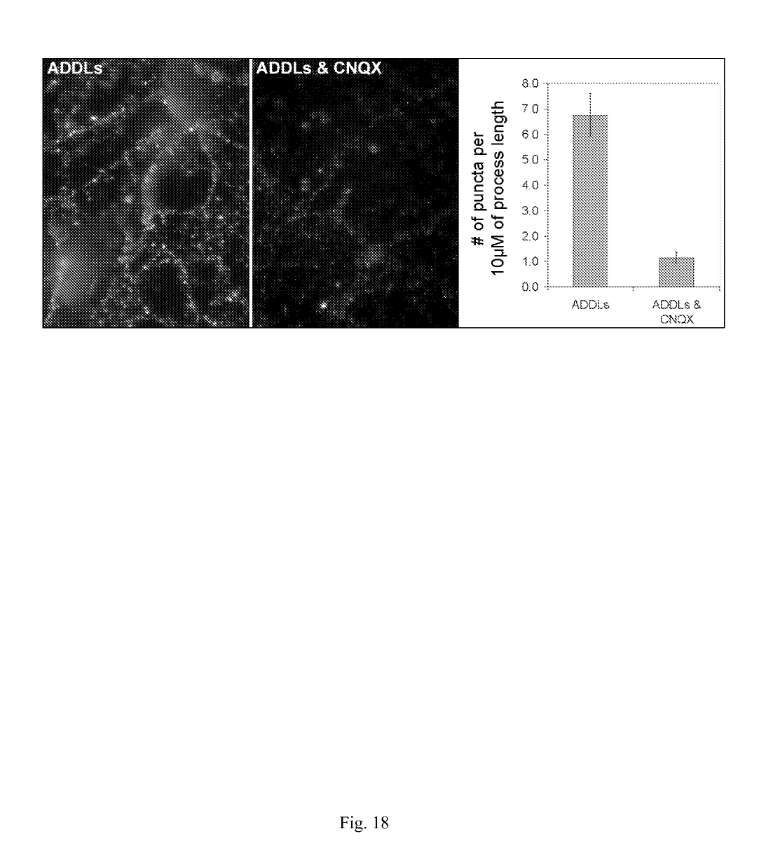

As shown in FIG. 18, CNQX blocks ADDL binding to the surface of neurons. ADDLs or ADDLs+CNQX were incubated with neuronal cells in culture as described berein. Typical ADDL punctate binding was observed and individual puncta were counted per a given process length. The number of ADDL punctate binding sites decreases in the presence of CNQX.

### EXAMPLE 7

### Quantification of ADDL Binding to Neurons

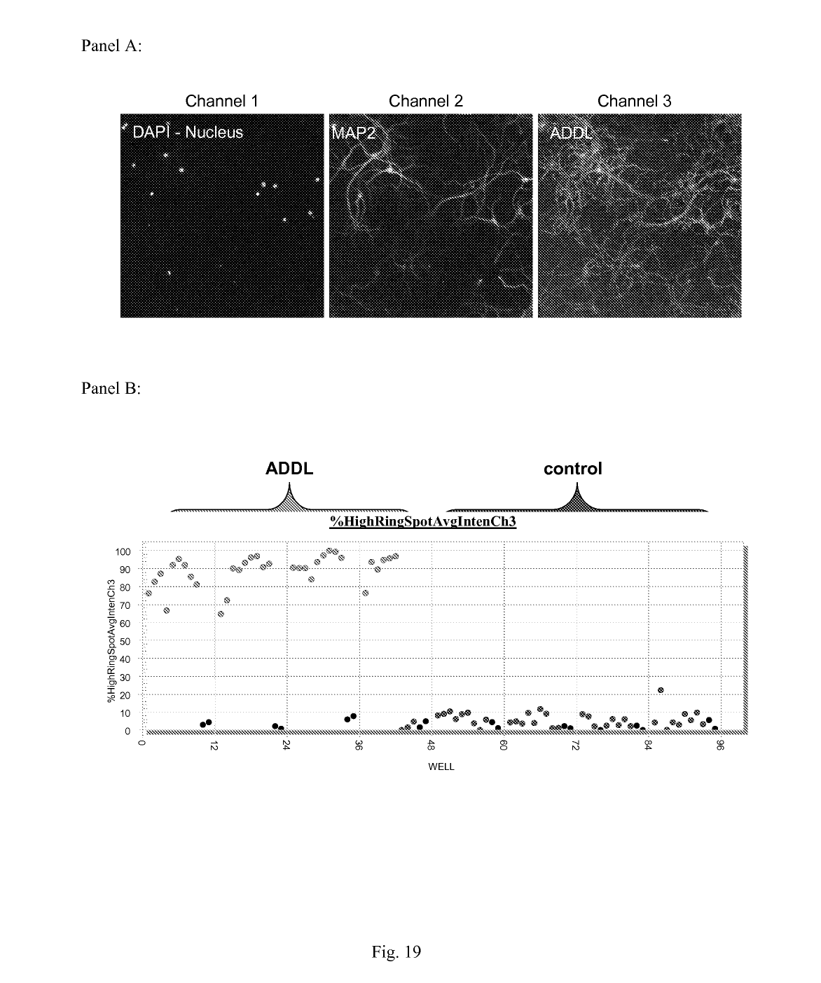

As shown in FIGS. 19 & 20, the binding of ADDLs to neurons can be quantified.

Biotinylated ADDLs were prepared according to standard protocols.

Increasing amounts of biotin-ADDLs (0.07 μM-17.8 μM) were added to primary hippocampal cultures and incubated for 15 min at 37 C. Neurons were subsequently washed with warm phosphate buffered saline (PBS) and fixed with 4% paraformaldehyde at 4 C for 20 min. Paraformaldehyde was removed by washing the cells several times with PBS. Non-specific binding was blocked using 2% BSA (bovine serum albumin) in PBS and incubation for 30 min at RT. Neurons were incubated with streptavidin coupled to alkaline phosphatase (Molecular Probes, 1:1500) for 1 h at room temperature. Non-specific binding was removed by washing the cells with PBS for several times. ADDL binding was detected using CDP Star with Sapphire-II as a substrate for alkaline phosphatase. End point luminescence was measured after 30 min incubation at room temperature using Tecan GENios pro. (see e.g., FIG. 19)

ADDL Binding Immunocytochemistry: Primary hippocampal neurons were incubated with 2.5 uM ADDLs for 15 min at 37 C. Neurons were subsequently washed with warm PBS and fixed with 4% paraformaldehyde for 15 min and subsequently washed with phosphate buffered solution (PBS, pH 7.4). Non-specific binding was blocked using 2% normal goat serum in PBS for 30 min at room temperature. Primary antibodies were incubated over night at 4 C (rabbit anti microtubule associated protein (MAP2) 1:700 dilution, and mouse anti ADDL antibody 1:2000 dilution. The following day cultures were washed with PBS and subsequently incubated with appropriate AlexaFluor 488 or 594 conjugated IgG (Molecular Probes, Eugene, Oreg.) (2 μg/ml) for 2 h at RT. In addition, DAPI nucleic stain was added at 300 nM in PBS for 30 min. Subsequently, cultures were washed four times with PBS and imaged using a Cellomics Arrayscan platform.

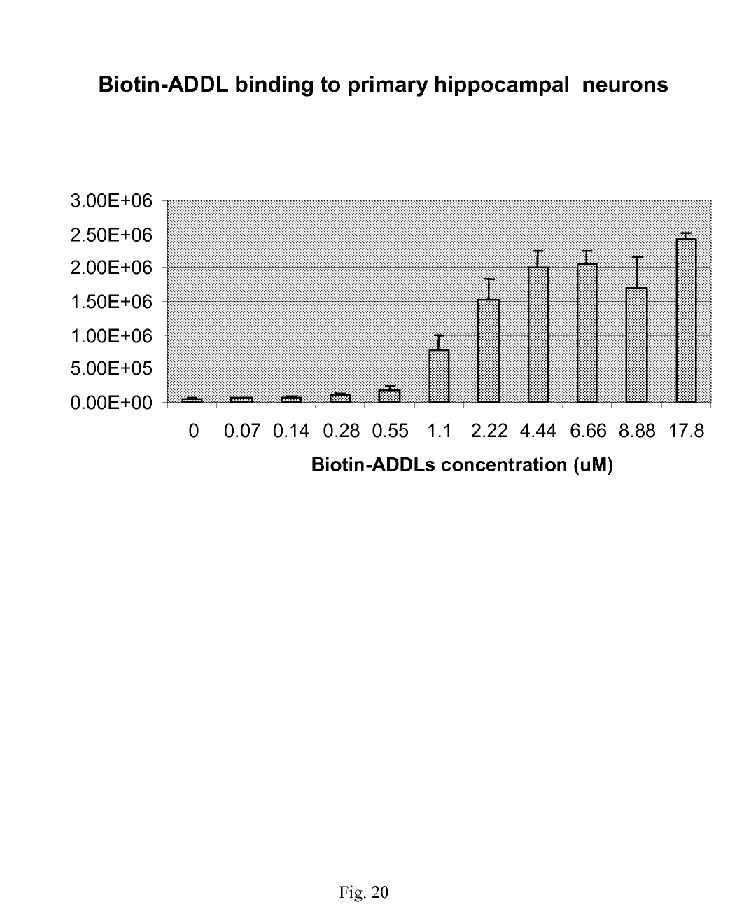

Arrayscan: A modification of the Arrayscan Compartmental Analysis BioApplication was used for image analysis of ADDL positive primary hippocampal cultures. Objects were identified using 3 channels for measurement of fluorescent intensity. Channel 1 was for the primary object (nucleus visualized via DAPI stain), and the average and total intensity for this object was measured. Channel 2 and 3 are dependent channels, whereby channel 2 was assigned to the neuronal MAP 2 staining (visualized by AlexaFluor 594) and channel 3 was assigned to the ADDL staining (visualized by AlexaFluor 488). Images were obtained with a 10× objective and a total of 15 fields per well were scanned. (see e.g., FIG. 20, Panels A & B)

### EXAMPLE 8

### ADDL Receptors

Membrane Preparation

(1) All manipulations were performed at 4° C., except as indicated in some steps. Whole brains were removed from adult rats on ice.

(2) The cerebellum, cortex, and hippocampus were separated in PBS. After dissection of the unwanted white matter, and removal of the large blood vessels.

(3) The coronal sections were washed with 3 vol Buffer A containing: PBS, pH 7.4 with 0.32 M sucrose, 50 mM HEPS, 25 mM MgCl₂, 0.5 mM dithiothreitol, 200 μg/ml PMSF, 2 μg/ml pepstatin A, 4 μg/ml leupeptin, and 30 μg/ml benzamidine hydrochloride for 3 times.

(4) 1 g tissue homogenization was in 20 vol Buffer A for 20 times, and the mixture was centrifuged at 1,000×g for 10 min.

(5) The pellet was resuspended in 15 vol Buffer A repeated step 4.

(6) The combined supernatant fluids were centrifuged at 100,000×g for 1 h.

(7) The pellet was suspended in 30 ml PBS and was centrifuged again 100,000×g for 45 minutes.

(8) The pellets were resuspended in 2 ml PBS and were used as cell membrane and kept at −83° C.

Enrich ADDL Receptor by Detergent Treatment and Linear Sucrose Gradient ultracentrifuge:

Detergent Treatment:

40 mg×6 cortex membrane protein for adult rat cortex were dissolved in 120 ml 5 mM Tris-HCl pH 9.5 containing 0.4% Zwittergent for 1 hour at RT.

Linear Sucrose Gradient Ultracentrifuge:

10 ml 5 mM Tris-HCl pH 7.4 containing 30-60% sucrose linear gradient was prepared and induced onto the bottom of one ultracentrifuge tube. 20 ml detergent treatment solution was applied onto the top of this sucrose linear gradient. The ultracentrifuge was run for 18 hours at 100,000 g. The pellets at the bottom were used as coarse sample containing p140 and p260. This sample was dissolved in 3 ml 10% SDS, and which was diluted again to 1% SDS by 10 mM sodium phosphate for 1 hour at RT. This solution was centrifuged at 100,000 g for 1 hour at 21° C. The supernatant was applied onto CHT HPLC.

Enrich ADDL Receptors by CHT-Column:

The supernatant (i.e., ADDLs receptors crude extract) was applied onto Econo-Pac CHT-II cartridge equilibrated 10 mM phosphate buffer (pH7.2), 1% SDS, and 0.5 mM DTT. After washing with the equilibration buffer, the chromatography was developed with a linear gradient of sodium phosphate (from 10 to 700 mM) in the same buffer. The buffers and the column were maintained at 28° C. to prevent SDS precipitation. 200 μl elution fractions were dialysed against 1% SDS 10 mM Tris-HCl pH 7.4 overnight. These fractions were concentrated to 60 μl by ultrifiltration with Centricon (Amicon, 10-kDa cut-off) and were concentrated again to 25 μl by 100% PEG.

Identify ADDL Receptors in Fractions from Column:

Synthetic ADDLs were used as ligand. Rat cortex 75 μg proteins were dissolved 30 μl Electrophoresis Sample Buffer for control. The concentrated fractions were mixed with 25 μl Electrophoresis Sample Buffer. Electrophoresis conditions: 4-20% Tris-HCl gel, 120 V, 1.5 h at RT and 2.5 in cold room. Transfer: 100 V 1 hour. The nitrocellulose membrane was blocked by 5% non-fat dry milk powder in TBS.T1 for overnight, and was washed by TBS.T1 3×15 min at RT. Proteins on nitrocellulose membrane were incubated with 10 nM sADDLs in 10 ml F12 Media for 3 hours in cold room. The nitrocellulose membrane was washed by TBS.T1 3×15 min at RT and incubated with primary antibody M71/2 1:4,000 in TBS.T1 with 5% milk for 1 hours at RT. The membrane was washed by TBS.T1 3×15 min at RT and incubated with second antibody Ig rabbit to M71/2 1:160,000 with 5% milk for 1 hours at RT, then washed by TBS.T1 3×15 min at RT. The image was developed by ECL, Femto Kit (0.5 ml each and 1.0 ml water).

Separate p140 and p260 by Electrophoresis:

The fractions containing p140 and p260 from CHT-column were concentrated and were separated by SDS-PAGE. The membrane proteins of control were transferred to nitrocellulose for sADDLs ligand blot. The gel of other lines was stained by Coomassie Blue R 250. After compared with control, the p140 and p260 were cut out and were sent to Michigan State University for sequencing.

LC-MS/MS or N-Terminal Sequence:

LC-MS/MS: Proteins in SDS-PAGE gel are stained with Coomassie blue R-250.

The bands are excised and protein is digested with trypsin in the gel, peptides eluted and fractioned by HPLC, then introduced into a mass spectrometer. Peptide sequences were searched in Mascot.

N-terminal sequence: After proteins were transferred to PVDF membrane, they were stained with Coomassie blue R-250. The protein bands were cut out, and the N-terminal sequences of proteins were run by Edman chemistry.

Two proteins identified as p140 and p260 have been further determined to be a protein called synGAP and a protein called ProSAP/Shank (see e.g. U.S. Pat. No. 6,723,838; Park, E. et al. (2003) J. Biol. Chem., vol. 278, no. 21, pp. 19220-19229; Roussignol, G. et al. (2005) J. Neurosci., vol. 25, no. 14, pp. 3560-3570; Sala, C. et al. (2005) J. Neurosci., vol. 25, no. 18, 4587-4592; Soltau, M. et al. (2004) J. Neurochem., vol. 90, pp. 659-665; references in any of the foregoing, and the like). These are scaffold proteins that exist in the post synaptic density (PSD) and serve to anchor various receptors and channels. ADDLs appear to interact with both. There are likely other transmembrane ADDL receptor protein(s) that are as yet unidentified. Such receptors can include, but are not limited to, post-synaptic density (PSD) receptors, glutamate receptors (e.g. mGluR, AMPA, NMDA, GluR2, GluR5, GluR6, and the like), sodium/potassium ATPase (i.e., Na⁺/K⁻ ATPase), integrin receptors, adhesion receptors, trophic factor receptors (e.g., trophin receptors), GABA receptors, CAM kinase, and the like (see e.g., U.S. Pat. No. 4,975,430; Wang, Q. et al. (2004) J. Neurosci., vol. 24, no. 13, pp. 3370-3378; Maj, M. et al. (2003) Neuropharmacol., vol. 45, no. 7, pp. 895-906; Blanchard, B. J. et al. (2002) Biochem. Biophys. Res. Comm., vol. 293, no. 4, pp. 1197-1203; Blanchard, B. J. et al. (2002) Biochem. Biophys. Res. Comm., vol. 293, no. 4, pp. 1204-1208; Allen, J. W. et al. (1999) Neuropharmacol., vol. 38, no. 8, pp. 1243-1252; Oka, A. & Takashima, S. (1999) Acta Neuropathol. (Berl.), vol. 97, no. 3, pp. 275-278; Copani, A. et al. (1995) Mol. Pharmacol., vol. 47, no. 5, pp. 890-897; Louzada, P. R. et al. (2001) Neurosci. Lett., vol. 301, pp. 59-63; Lavreysen, H. et al. (2003) Mol. Pharmacol., vol. 63, no. 5, pp. 1082-1093; Conquet, F. et al. (1994) Nature, vol. 372, pp. 237-243; Battaglia, G. et al. (2001) Mol. Cell. Neurosci., vol. 17, pp. 1071-1083; Bruno, V. et al. (2001) vol. 21, pp. 1013-1033; references in any of the foregoing; and the like).

### EXAMPLE 9

### synGAP, shank3, and Glutamate Receptors

### Synapses in Early Alzheimer's Disease

Amyloid β [beta] (Abeta) peptides that are released from presynaptic sites in the dentate gyrus and deposited in extracellular plaques can have an effect on synaptic function (see e.g., Lazarov, O. et al. (2002) J. Neurosci., vol. 22, pp. 9785-9793; references therein; and the like). There is a significant loss of synaptic connectivity and of synaptic vesicles, as well as a change of synaptic numbers and synaptic function in many regions of the neocortex and hippocampus in brains identified as being afflicted with Alzheimer's disease (AD) (see e.g., Scheff, S. W. & Price, D. A. (2003) Neurobiol. Aging, vol. 24, pp. 1029-1046; Coleman, P. D. & Yao, P. J. (2003) Neurobiol. Aging, vol. 24, pp. 1023-1027; reference in either of the foregoing; and the like). Synaptic density is decreased by about 50% in AD brains (see e.g. Brun, A. et al. (1995) Neurodegeneration, vol. 4, pp. 171-177; references therein; and the like).

### Soluble Amyloid and Alzheimer's Disease

Aβ (Abeta) is synaptotoxic in the absence of plaques (see e.g. Mucke, L. et al. (2000) J. Neurosci., vol. 20, pp. 4050-4058; references therein; and the like). Alterations of hippocampal synaptic efficacy prior to neuronal generation, and that the synaptic dysfunction is caused by diffusible oligomeric assemblies of the amyloid beta protein (see e.g., Selkoe, D. J. (2002) Science, vol. 298, pp. 789-791; references therein; and the like). Water soluble oligomers of β [beta] amyloid peptides 1-40 and 1-42 exist in cerebral cortex of normal and Alzheimer's disease brains. AD brains contain more water soluble Aβ (Abeta) than control brains (see e.g., Kuo, Y. M. (1996) J. Biol. Chem., vol. 271, pp. 4077-4081; references therein; and the like). Concentrations of soluble Abeta from AD patients are a strong correlate of synapse loss (see e.g., Lue, L. F. et al. (1999) Am. J. Pathol., vol. 155, pp. 853-862; references therein; and the like). LRP may contribute to memory deficits typical of Alzheimer's disease by modulating the pool of small soluble forms of Abeta (see e.g., Zerbinatti, C. V. et al. (2004) Proc. Nat'l. Acad. Sci. USA, vol. 101, pp. 1075-1080; references therein; and the like).

### ADDLs in Alzheimer's Disease

Self-assembly of Abeta (1-42) forms globular, neurotoxic ADDLs (see e.g. Chromy, B. A. et al. (2003) Biochemistry, vol. 42, pp. 12749-12760; references therein; and the like). ADDL impaired synaptic plasticity and associate memory dysfunction during early stage Alzheimer's disease and lead to cellular degeneration and dementia during end stage (see e.g., Lambert, M. P. et al. (1998) Proc. Nat'l. Acad. Sci. USA, vol. 95, pp. 6448-6453; references therein; and the like). Oligomeric Abeta ligands (ADDLs; amyloid P derived diffusible ligands) were increased in AD frontal cortex to 70 times (see e.g., Gong, Y. S. et al. (2003) Proc. Natl. Acad. Sci. USA, vol. 100, pp. 10417-10422; references therein; and the like). Targeting small Abeta oligomers can be a solution to the Alzheimer's disease conundrum (see e.g., Klein, W. L. et al. (2001) Trends Neurosci., vol. 24, pp. 219-224; references therein; and the like).

### Glutamate Receptors in Alzheimer's Disease

Multiple neuroreceptor changes are present in Alzheimer's disease. Interestingly, kainite receptors increase in number while NMDA receptors are reduced in cortical Alzheimer's brain tissue. The muscarinic (M1), kainite, and CRF receptors show receptor compensatory reactions probably due to degenerative reactions in Alzheimer's disease (see e.g., Guan, Z. Z. et al. (2003) J. Neurosci. Res., vol. 71, no. 3, pp. 397-406; Nordberg, A. et al. (1992) J. Neurosci. Res., vol. 31, no. 1, pp. 103-111; Nordberg, A. (1992) Cerebrovasc. Brain Metab. Rev., vol. 4, no. 4, pp. 303-328; references in any of the foregoing; and the like).

### Glutamate Receptors

The glutamate receptors are both seven transmembrane domain G protein-coupled receptors (metabotropic) and ligand-gated ion channels (ionotropic). The ionotropic receptors cluster into three definable families: the NMDA type, the AMPA type (e.g., GluR1, GluR2, GluR3, and GluR4), as well as the kainate type (e.g., GluR5, GluR6, and GluR7). These receptors are multimeric associations of specific subunits and have specific binding domains on the final receptor complexes (see e.g. Meador-Woodruff, J. H. et al. (2003) Ann. N.Y. Acad. Sci., vol. 1003, pp. 75-93; references therein; and the like).

### Kainate Receptor Subunits Homology

GluR5 was the first mammalian kainate receptor subunit to be cloned, showing about 40% sequence homology to the AMPA receptor subunits GluR1-GluR4. Another four kainate receptor subunits (GluR6, GluR7, KA1 and KA2) can be divided into two groups on the basis of their structural homology and affinity for [³H]kainate. Kainate receptor complexes are formed from five different protein subunits including KA1 and KA2 (high affinity kainate preferring) and GluR5-GluR7 (low affinity kainate preferring). The low affinity subunits, GluR5-GluR7 display about 75% homology, while the high-affinity subunits, KA1 and KA2, are about 68% homologous. The homology between GluR5-GluR7 and KA1/KA2 is much lower at about 45%. As with the AMPA receptor subunits, each of the kainate receptor subunits comprises about 900 amino acids with a relative molecular weight (Mᵣ) of about 100 kDa (see e.g., Chittajallu, R. et al. (1999) Trends Pharmacol. Sci., vol. 20, no. 1, pp. 26-35; references therein; and the like).

### Kainate Receptors and Long Term Potentiation (LTP)

Kainate receptors play a role in the induction of long-term potentiation (LTP) at mossy fiber synapses in the hippocampus. In kainate receptor knock-out mice, LTP is reduced in mice lacking the GluR6, but not the GluR5, kainate receptor subunit. These facts demonstrate that kainate receptors containing the GluR6 subunit are important modulators of mossy fiber synaptic strength (see e.g., Contractor, A. et al. (2001) Neuron, vol. 29, pp. 209-216; references therein; and the like).

Glutamate Receptors, synGAP, and the Post Synaptic Density (PSD)

In the case of both the ionotropic and the metabotropic receptors, intracellular proteins associated with the postsynaptic density have been identified that have specific associations with both types of receptors. PSD 95 has specific associations with NMDA (NR2) and GluR5,6/KA2 (see e.g., Meador-Woodruff, J. H. et al. (2003) Ann. N.Y. Acad. Sci., vol. 1003, pp. 75-93; Hirbec, H. et al. (2003) Neuron, vol. 37, pp. 625-638; references in either of the foregoing; and the like).

SynGAP is selectively expressed in brain and is highly enriched at excitatory synapses, where it is present in a large macromolecular complex with PSD 95 and the NMDA receptor. SynGAP stimulates the GTPase activity of Ras, suggesting that it negatively regulates Ras activity at excitatory synapses. Ras signaling at the post-synaptic membrane may be involved in the modulation of excitatory synaptic transmission by NMDA receptors and neurotrophins (see e.g. Kim, J. H. et al. (1998) Neuron, vol. 20, pp. 683-691; references therein; and the like). At the post-synaptic membrane of excitatory synapses, neurotransmitter receptors are attached to large protein “signaling machines,” the post-synaptic density that contributes to information processing and the formation of memories (see e.g., Kennedy, M. B. (2000) Science, vol. 290, pp. 750-754; Walikonis, R. S. et al. (2000) J. Neurosci., vol. 20, no. 11, pp. 4069-4080; references in either of the foregoing; and the like).

At excitatory synapses, the postsynaptic scaffolding protein postsynaptic density 95 (PSD 95) couples with NMDA receptors (NMDARs) to the Ras GTPase-activating protein synGAP (see e.g., Komiyama, N. H. et al. (2002) J. Neurosci., vol. 22, pp. 972109732; references therein; and the like). The regulation of synaptic Ras signaling by synGAP is important for proper neuronal development and glutamate receptor trafficking and is critical for the induction of LTP. In mutant mice, without proper regulation of Ras by synGAP, activated Ras at synapses can lead to increased Ras signaling, including activation of the MAP kinase cascade (see e.g., Kim, J. H. et al. (2003) J. Neurosci., vol. 23, pp. 1119-1124; references therein; and the like). SynGAP also regulates ERK/MAPK signaling (see e.g., Komiyama, N. H. et al. (2002) J. Neurosci., vol. 22, pp. 972109732; references therein; and the like). Inhibition of synGAP by CaMKII will stop inactivation of GTP-bound Ras and could result in activation of the mitogen-activated protein (MAP) kinase pathway in hippocampal neurons upon activation of NMDA receptors (see e.g. Chen, H. J. et al. (1998) Neuron, vol. 20, pp. 895-904; Komiyama, N. H. et al. (2002) J. Neurosci., vol. 22, pp. 972109732; references in either of the foregoing; and the like).

### ADDLs, Shank3 and Glutamate Receptors

In neuronal cells, Shank proteins localize to postsynaptic densities (PSDs) and have been shown to regulate dendritic spine morphology by linking the postsynaptic signaling machinery to the cortical cytoskeleton (Naisbitt et al., 1999; Tu et al., 1999; Sheng and Kim, 2000; Sala et al., 2001; Boeckers et al., 2002). Glutamate receptors are key elements of the post-synaptic signaling machinery and the shank proteins establish a linkage between the mGluRs and the GluRs via other PSD scaffold proteins such as PSD-95, GKAP and the homer family of proteins. ADDLs are capable of binding to ProSAP2/shank3, the p260 protein band isolated from hippocampal synaptosomes and identified by mass spectrometry. ADDL binding to the complex of shank3 and either of the group I mGlu receptors mGluR1 and mGluR5 may trigger mGlu signaling, thereby interfering with LTP (Wang et al., 2004).

### ADDLs and LTP

ADDLs impair synaptic plasticity and inhibit LTP during early stage Alzheimer's disease and can lead to cellular degeneration and dementia during end stage (see e.g. Lambert, M. P. et al. (1998) Proc. Natl. Acad. Sci. USA, vol. 95, pp. 6448-6453; references therein; and the like). Oligomers of amyloid beta protein potentially inhibit hippocampal long-term potentiation in vivo (see e.g., Walsh, D. M. et al. (2002) Nature, vol. 416, pp. 535-539; references therein; and the like). Soluble oligomers of Abeta (1-42) inhibit long-term potentiation, but not long-term depression in rat dentate gyrus (see e.g., Wang, et al. (2002) Brain Res., vol. 924, pp. 133-140; references therein; and the like).

Other background information includes, but is not limited to, U.S. Pat. No. 6,811,992; U.S. Pat. No. 6,723,838; U.S. Pat. No. 6,653,102; U.S. Pat. No. 6,515,107; U.S. Pat. No. 6,500,624; U.S. Pat. No. 6,228,610; U.S. Pat. No. 6,221,609; U.S. Pat. No. 6,051,688; U.S. Pat. No. 6,040,175; U.S. Pat. No. 6,033,865; U.S. Pat. No. 5,912,122; U.S. Pat. No. 5,888,996; U.S. Pat. No. 5,783,575; U.S. Published Patent App. No. 2003/0176651; Fleck, M. W. et al. (2003) J. Neurosci., vol. 23, no. 4, pp. 1219-1227; Meldrum, B. S. (2000) J. Nutr., vol. 130, pp. 1007S-1015S; Senkowska, A. & Ossowska, K. (2003) Pol. J. Pharmacol., vol. 55, no. 935-950; Ronnback, L. & Hansson, E. (2004) J. Neuroinflammation, vol. 1, no. 1, pp. 22-30; Lee, J.-M. et al. (2000) J. Clin. Invest., vol. 106, no. 6, pp. 723-731; and Tao, H. W. et al. (2001) Proc. Nat'l. Acad. Sci. USA, vol. 98, no. 20, pp. 11009-11015; references in all of the foregoing; and the like.

Two proteins, p140 and p260, can bind ADDLs with high affinity, both are found only in the cortex and hippocampus. From mass spectroscopy (MS) data, 55 peptides from p140 match synGAP in PSD. The molecular size of p140 approximates the molecular size of synGAP. In immunocytochemistry experiments, ADDL “hotspots” are co-localized with synGAP. When ADDLs are initially incubated with p140 on nitrocellulose, the ADDLs can block the binding of an N-terminal specific antibody to synGAP. However, ADDLs cannot block the binding of a C-terminal specific antibody to synGAP under similar conditions. This demonstrates that ADDLs can bind to synGAP, likely at or near the N-terminus of synGAP, and block or cover one or more epitopes of an N-terminal antibody (see e.g., Lacor, P. et al. (2004) J. Neurosci., vol. 24, pp. 10191-10200; and references therein.

Homologous Sequence of synGAP and Glutamate Receptors

Disclosed herein is a previously unrecognized sequence homology between synGAP (SEQ ID No. ______) and glutamate receptors (SEQ ID Nos. ______ & ______):

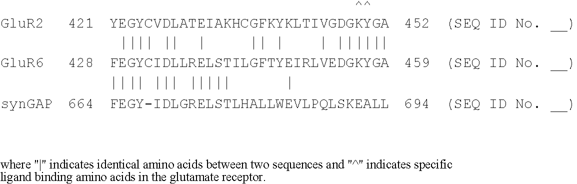

When the same regions are aligned using the ClustalW algorithm, the alignment is:

| | |
|---|---|
| GluR2 | YEGYCVDLATEIAKHCGFKYKLT--IVGDGKYGA |
| GluR6 | FEGYCIDLLRELSTILGFTYEIR--LVEDGKYGA |
| synGAP | FEGY-IDLGRELSTLHALLWEVLPQLSKEALL-- |
| | :*** :** *::. .: ::: : :. |
| consensus | FEGYCIDL-RELST--GF-YE--PQLV-DGKYGA |

where in the consensus “*” indicates identical amino acids, “:” indicates strongly similar amino acids, and “.” indicates weakly similar amino acids.

A similar homology exists when the sequence of same region of the GluR5 precursor protein (accession no. P39086 at N.C.B.I. Entrez Protein) is added to the alignment:

| | |
|---|---|
| GluR2 | YEGYCVDLATEIAKHCGFKYKLT--IVGDGKYGA |
| GluR5 | FEGYCLDLLKELSNILGFIYDVK--LVPDGKYGA |
| GluR6 | FEGYCIDLLRELSTILGFTYEIR--LVEDGKYGA |
| synGAP | FEGY-IDLGRELSTLHALLWEVLPQLSKEALL-- |
| | :*** :** *::. .: :.: : :. |
| Consensus | FEGYCIDLLRELSTILGF-YEV-PQLV-DGKYGA |

again where in the consensus “*” indicates identical amino acids, “:” indicates strongly similar amino acids, and “.” indicates weakly similar amino acids.

This homology is localized to the ligand binding region of the glutamate receptor, which can be an indication that ADDLs bind to the homology sequence in glutamate receptors, thereby inhibiting LTP. Considering the representation of a crystal structure of glutamate receptor (GluR2SIS2) bound to kainate as shown in Armstrong, N. et al. (1998) Nature, vol. 395, pp. 913-917), the region of homology between glutamate receptor and synGAP would be near the J helix of the GluR2 S1S2 crystal structure.

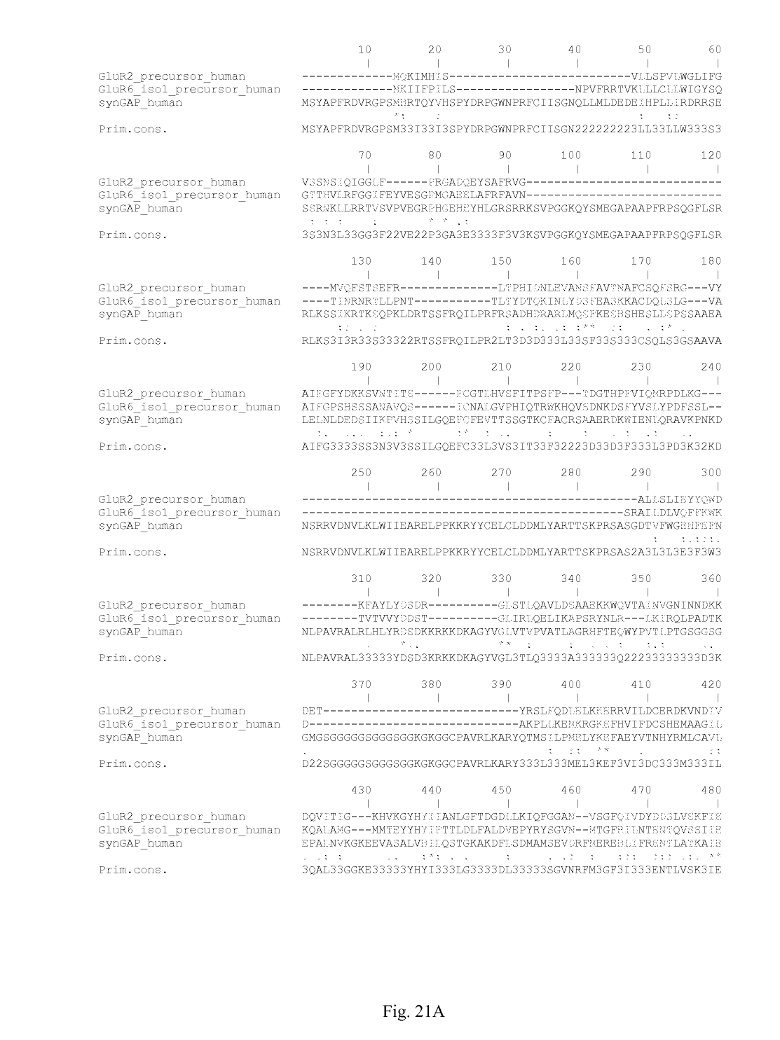

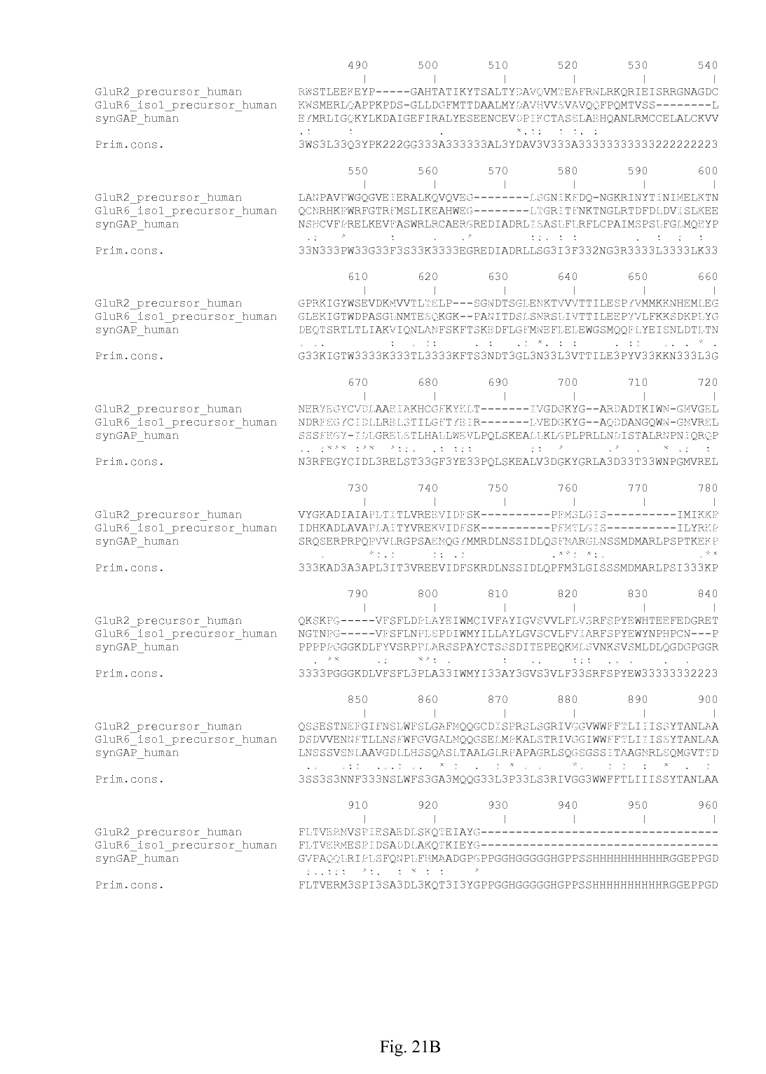

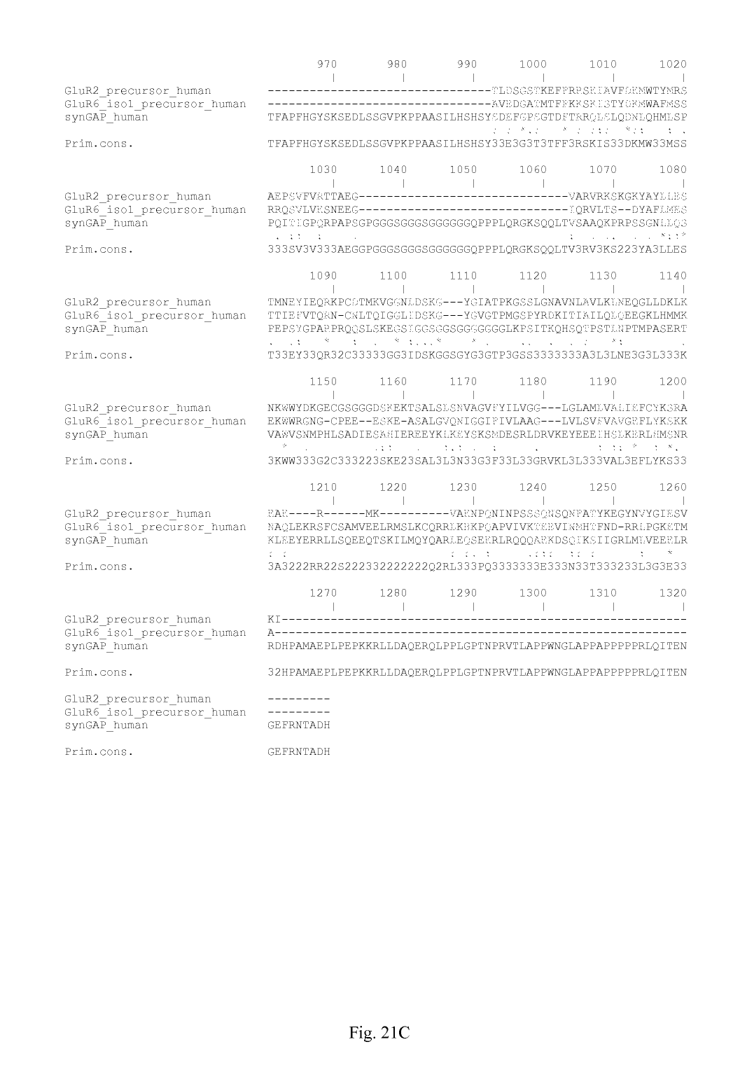

FIG. 21 (panels A-C) shows the results of a ClustalW alignment of the sequences of human synGAP (accession nos. NP_{—}006763 and Q96PVO at N.C.B.I. Entrez Protein), human glutamate receptor 2 precursor (accession no. P42262 at N.C.B.I. Entrez Protein), and human glutamate receptor 6 isoform 1 precursor (accession no. NP_{—}068775 at N.C.B.I. Entrez Protein). Sequence alignment performed by NPS@: Network Protein Sequence Analysis, Combet, C. et al. (2000) TIBS, vol. 25, no. 3, pp. 147-150 (<http://npsa-pbil.ibcp.fr/cgi-bin/npsa_automat.p1?page=npsa_clustalw.html>; last visited Dec. 15, 2004).

Glutamate and Glutamate Receptor Ligands CNQX and NS-102 Block ADDL Binding to Dendritic Receptors.

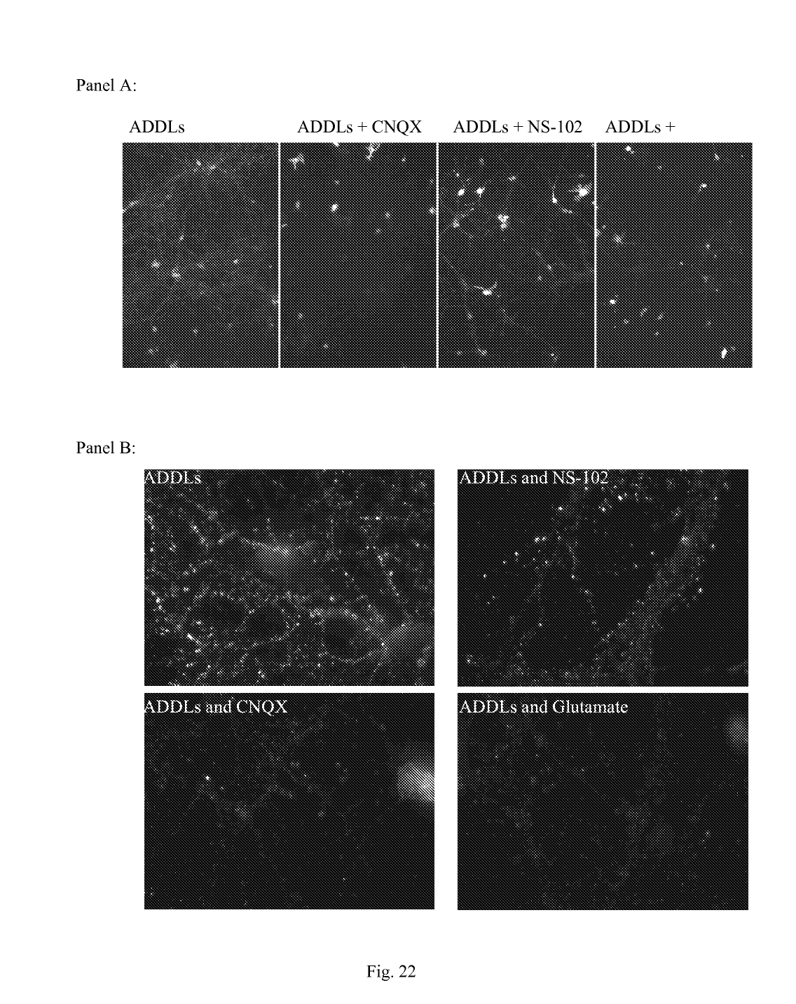

ADDL binding to post-synaptic localized receptors or receptor complexes can be blocked by the addition of glutamate or the glutamate receptor ligands CNQX and NS-102, as shown in FIG. 22, Panels A & B. The diminished ADDL binding results because ADDLs are binding directly to one or more of the glutamate receptors, or because the glutamate receptor ligands induce a change via the glutamate receptors that reduces the binding affinity of the ADDL receptor for ADDLs.

Glutamate receptors fall into two classes, metabotropic and ionotropic. The Group I mGlu receptors localized to postsynaptic sites are mGluR1 and mGluR5, and it is likely that ADDLs bind directly to these receptors or to a complex that includes these receptors and other post-synaptic density-anchored proteins.

The ionotropic glutamate receptors (GluRs) are gated ion channels and include the AMPA and kainite receptors. These are tetrameric assemblies containing GluR1-4 subunits and GluR5-7 subunits, respectively. The exact combination of different subunits within the functional tetrameric channels determines the particular binding and ion transport characteristics. ADDLs are most likely to bind to the AMPA receptors, in view of the blocked synaptic binding by the glutamate ligands, however, ADDL binding to the ADDL receptor also could be blocked indirectly due to conformational changes in the ADDL receptor triggered by ligand engagement with the GluRs and subsequent indirect effects on the ADDL receptor.

ADDLs are known to bind to the post-synaptic density anchored protein SHANK3, a protein that is known to interact directly with the mGluR5 receptor.

### Immunofluorescence Examination of Effects of GluR Blockers on ADDL Binding to Hippocampal Cells.

Previous experiments showed GluR6 co-localized, in part, with ADDLs and glutamine blocked, at least in part, ADDL binding to synaptosomes. Therefore, the ability of GluR blockers to block ADDL binding to neuronal cells was assessed.

Hippocampal cells were plated on poly-L-lysine coated coverslips and grown by Sara for 25 days. ADDLs were made by Daliya on Sep. 21, 2004, concentration of 54.2 μM. L-Glutamate (5 mM), NS-102 (50 μM), CNQX (100 μM), or nothing was added to culture dishes followed immediately by addition of ADDLs (0.5 μM) and incubated for 15 min at 37° C. Vehicle was added to one dish as control. Cells were fixed by adding an equal volume of 3.7% formaldehyde to the media for 5 minutes followed by the removal of the entire fix:media solution and replacement with 3.7% formaldehyde only for 10 minutes. Cells were rinsed 4 times with PBS, then incubated with PBS: 10% NGS overnight at 8° C. Cells were immunolabeled with 20C2 (1:1000) diluted in PBS:NGS for 3 hours at room temperature. Cells were rinsed 4 times with PBS, then incubated with Alexa Fluor 488 anti-mouse (1:500), diluted in PBS:NGS, for 3 hours at room temperature. Cells were rinsed 5 times with PBS and mounted with ProLong anti-fade mounting media. Cells were visualized live on the Nikon with MetaMorph. Results: CNQX and glutamate severely diminish ADDL binding to hippocampal cells. NS-102 shows some decrease in ADDL binding.

Glutamate is the ligand for three major classes of ionotropic and three major classes of metabotropic receptors that play a major role in excitatory neurotransmission and are required for LTP generation and normal brain function (Meldrum 2000).

Glutamate also binds to two glial transporters (GLAST and GLT) and three neuronal transporters (EAAC1, EAAT4 and 5) that play a major role in protecting against neurodegeneration (Kanai and Hediger 2003).

Glutamate binds to its substrates with a variety of affinities ranging from high (e.g., high affinity Na⁺-dependent glutamate transporters (Km=5-20 uM)) to low (low affinity glutamate transporters (1-2 mM)) (see Table 1).

5 mM glutamate was able to inhibit ADDL binding to neurons. The high concentration implies that ADDLs have a much higher affinity for the same sites that glutamate binds to.

TABLE 1
Glutamate: concentrations and affinities¹ (B.S. Meldrum 2000)
Approximate concentration in

| | | |
|---|---|---|
| CSF | <1 | μmol/L |
| Brain ECF | 0.5-2 | μmol/L |
| Plasma | 30-100 | μmol/L |
| Synaptic cleft | 2-1,000 | μmol/L |
| Brain (homogenate) | 10 | mmol/L |
| Synaptic vesicle | 100 | mmol/L |
| “Affinity“ (ED₅₀) | | |
| GLT-1 | 1-20 | μmol/L |
| NMDAR | 2.5-3 | μmol/L |
| mGluR2,3,4,8 | 5 | μmol/L |
| mGluR1,5 | 10 | μmol/L |
| AMPAR | 200-500 | μmol/L |
| mGluR7 | 1,000 | μmol/L |

¹CSF, cerebrospinal fluid; ECF, extracellular fluid; ED₅₀, 50% effective dose; GLT, rat glial glutamate transporter; NMDAR, N-methy-D-aspartate receptor; mGluR, metabotropic glutamate receptor; AMPAR, α-amino-3-hydroxy-5-methyl-4-isoxazoleproprionic acid receptor.

### An Immunofluorescence Examination of the Effects of Glutamate Receptor (GluR) Blockers on ADDL Binding to Hippocampal Cells

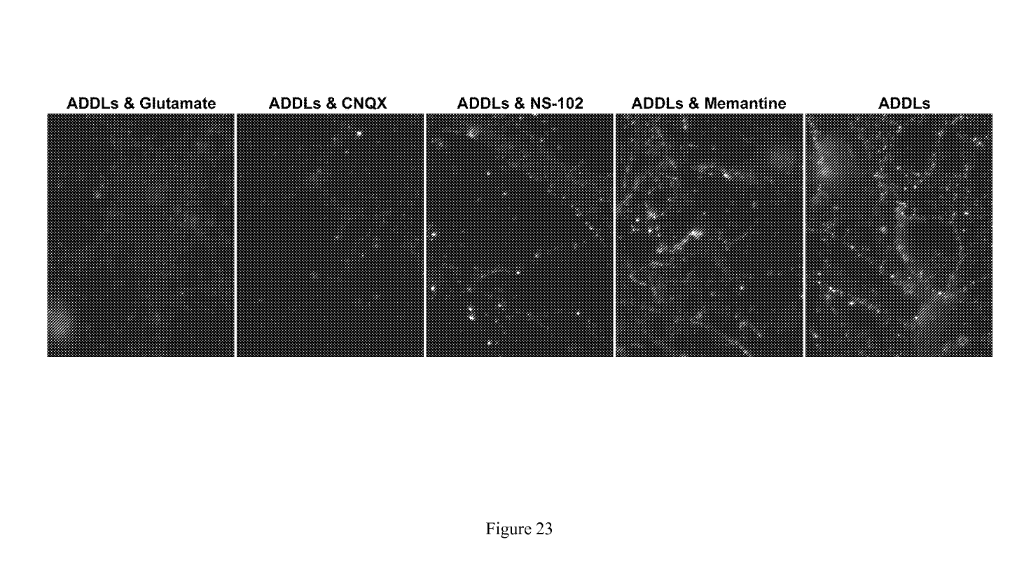

As disclosed previously, FIG. 23 shows that punctate ADDL binding to neurons in hippocampal cultures (previously shown to be synaptic binding) is blocked by glutamate and CNQX, a known antagonist of AMPA and kainate-type glutamate receptors. Previous experiments have shown that GluR6 colocalized in part with ADDLs and glutamine blocked, at least in part, ADDL binding to synaptosomes. Therefore, an examination was undertaken to determine whether GluR blockers could block ADDL binding to cells. Hippocampal cells were grown for 25 days under standard conditions. L-Glutamate (5 mM), CNQX (100 μM), NS-102 (50 μM), Memantine (50 μM), or nothing was added to the culture medium in separate dishes followed immediately by addition of ADDLs (0.1 μM) and incubated for 15 min at 37° C. Vehicle was added to one dish as control. Cells were fixed and immunolabeled with a monoclonal antibody specific for ADDLs (20C2) followed by Alexa Fluor 488 anti-mouse antibody. Cells were visualized using a Nikon Optiphot with epifluorescent attachment and MetaMorph Imaging software. Data shows that glutamate and CNQX are effective at blocking ADDL binding, NS-102 shows a partial block, and memantine shows a negligible effect on ADDL binding.

5 mM Glutamate Blocks/Prevents ˜75% of 100 nM ADDLs from Binding to Synaptosomes in Panning Assay

Parameters: Synaptosomes were sequentially bound to glutamate, ADDLs, and 20C2 monoclonal antibody, incubated in assay plate wells coated with anti-mouse IgG, and probed for 20C2 antibody.

Rationale: As disclosed herein, there is sequence homology between GluR6 and SynGAP. It is possible that ADDLs bind to a glutamate receptor. Therefore, it was determined whether ADDL binding can be blocked with glutamate.

Action: Goat anti-mouse IgG, Fc fragment specific (Jackson), was diluted to 10 mg/ml with 50 mM Tris-HCl, pH 9.5 and 100 ml/well (1 mg) allowed to bind to Immulon 4 Removawell strips (Dynatech Labs) for 7 hr at RT. Unbound sites were blocked with 3×200 ml 2% BSA in TBS (20 mM Tris-HCl, pH 7.5, 0.8% NaCl)×10 min at RT. Synaptosomes were mixed with 1 ml/tube of 1% BSA in F12 and centrifuged at 5,000 g×5 min at 4° C.; each pellet was washed with 1 ml of BSA/F12 and resuspended in 1 ml BSA/F12. Synaptosomes were divided and 5 mM glutamate in 1 ml of BSA/F12 was added to one tube and incubated for 2 hr at RT. Unbound glutamate was washed 3×1 ml of BSA/F12. Synaptosomes were divided again and 100 nM ADDLs in BSA/F12 were added to synaptosomes bound and not bound to glutamate and incubated for 1 hr at 37° C. The samples were pelleted, as above, and washed with 3×1 ml BSA/F12. Each pellet was resuspended in 1 ml BSA/F12 containing 1.52 mg monoclonal 20C2 IgG. The samples were placed on a rotating shaker and incubated for 2 hr at 4° C. The samples were pelleted, as above, and washed with 3×1 ml BSA/F12. Each pellet was resuspended with 220 ml BSA/F12, 100 ml/well added to the prepared assay plate and incubated overnight at 4° C. The wells were washed 3×200 ml×10 min with BSA/TBS. HRP-linked anti-mouse IgG (Amersham) was diluted 1:2000 in BSA/TBS and incubated 100 ml/well for 1 hr at RT. Following washing with BSA/TBS as above and rinsing 3× under running dH₂O, binding was visualized using 100 ml/well of Bio-Rad peroxidase substrate. Color was developed at RT and read at 405 nm on a Dynex MRX Microplate Reader. Statistics: Data is shown as the mean of duplicate values with error bars representing SEM.

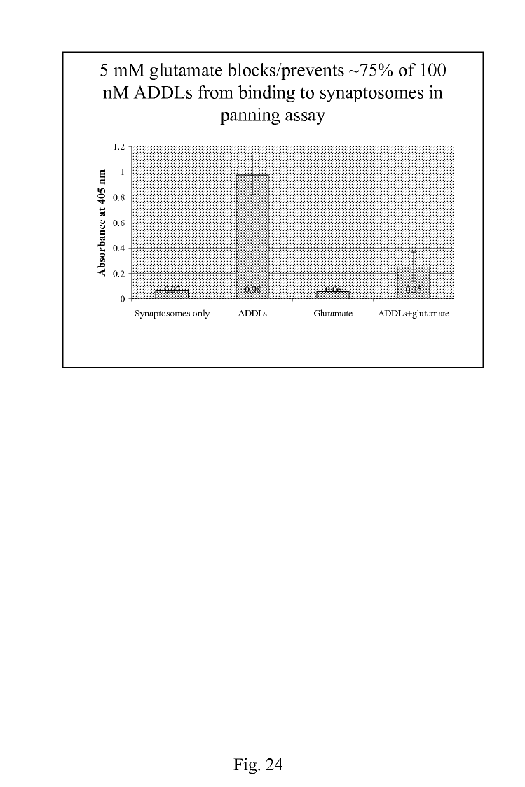

Results: As shown in FIG. 24, synaptosomes not labeled with ADDLs showed low background binding. Despite some deterioration in synaptosomes due to repeated washing and centrifuge, synaptosomes bound to ADDLs showed good signal at 15 min and 30 min (30 min data shown). There is a substantial (˜75%) decrease in ADDL signal when glutamate is present.

Established: The presence of glutamate has an effect on ADDL binding to synaptosomes in the panning assay. Without being bound by any one possible mechanism, glutamate can be directly blocking ADDL binding to one or more glutamate receptors or glutamate can be affecting and/or modifying ADDL receptors. As disclosed herein throughout, these results show that a relation between glutamate and ADDL binding exists.

### syn GAP/Glutamate Receptor Sequence Homology to Treat Alzheimer's Disease

The homologous sequence between synGAP and glutamate receptor as disclosed herein can be used to treat Alzheimer's disease by blocking the neurotoxicity of ADDLs. Peptides, protein fragments, and the like, which comprise the homologous sequence disclosed herein, can be used to block the binding of ADDLs to neurons, thereby preventing or treating Alzheimer's disease.

A target for anti-ADDL therapeutics can comprise glutamate receptors, which include kainate, AMPA, and NMDA subtypes. The GluR6 sub-type, a so-called kainate receptor, is illustrative of a receptor sub-type with a sequence homology to synGAP. Other sequence homologies also exist within AMPA receptors (e.g., GluR2) and NMDA receptors.

### EXAMPLE 10

### ADDL—Synaptosome Binding

Synaptosome Panning Shows that ADDL Binding is Dependent on Synaptosome Concentration

Parameters: Synaptosomes were labeled sequentially with ADDLs and monoclonal 20C2 antibody (see e.g. U.S. Patent No. 60/621,776, filed 25 Oct. 2004), incubated in assay plate wells coated with anti-mouse IgG, and probed for 20C2 antibody.

Rationale: Previous synaptosome panning results showed that synaptosomes labeled with ADDLs could be captured in antibody-coated wells. Occasionally, background fluorescent signal was present, perhaps due to the choice of plate (i.e., not an ELISA plate).

Action: Goat anti-mouse IgG, Fc fragment specific (Jackson), was diluted to 10 mg/ml with 50 mM Tris-HCl, pH 9.5 and 100 ml/well (1 mg) allowed to bind to Immulon 3 Removawell strips (Dynatech Labs) for 7 hr at RT. Unbound sites were blocked with 3×200 ml 2% BSA in TBS (20 mM Tris-HCl, pH 7.5, 0.8% NaCl)×10 min at RT. Synaptosomes were mixed with 1 ml/tube of 1% BSA in F12 and centrifuged at 5,000 g×5 min at 4° C.; each pellet was washed with 1 ml of BSA/F12 and resuspended in 1 ml BSA/F12. ADDLs were added (50 nM, 100 nM, and 200 nM) to tubes and the synaptosomes incubated for 1 hr at 37° C. The samples were pelleted, as above, and washed with 3×1 ml BSA/F12. Each pellet was resuspended in 420 ml of BSA/F12 and 200 ml aliquots mixed with 800 ml BSA/F12 containing 1.52 mg monoclonal 20C2 IgG. The samples were placed on a rotating shaker and incubated for 2 hr at 4° C. The samples were pelleted, as above, and washed with 3×1 ml BSA/F12. Each pellet was resuspended with 220 ml BSA/F12, 100 ml/well added to the prepared assay plate and incubated overnight at 4° C. Monoclonal 20C2 (1.5-15 ng/100 ml) diluted in BSA/F12 was also incubated in prepared wells. The wells were washed 3×200 ml×10 min with BSA/TBS. HRP-linked anti-mouse IgG (Amersham) was diluted 1:2000 in BSA/TBS and incubated 100 ml/well for 1 hr at RT. Following washing with BSA/TBS as above and rinsing 3× under running dH2O, binding was visualized using 100 ml/well of Bio-Rad peroxidase substrate. Color was developed at RT and read at 405 nm on a Dynex MRX Microplate Reader.

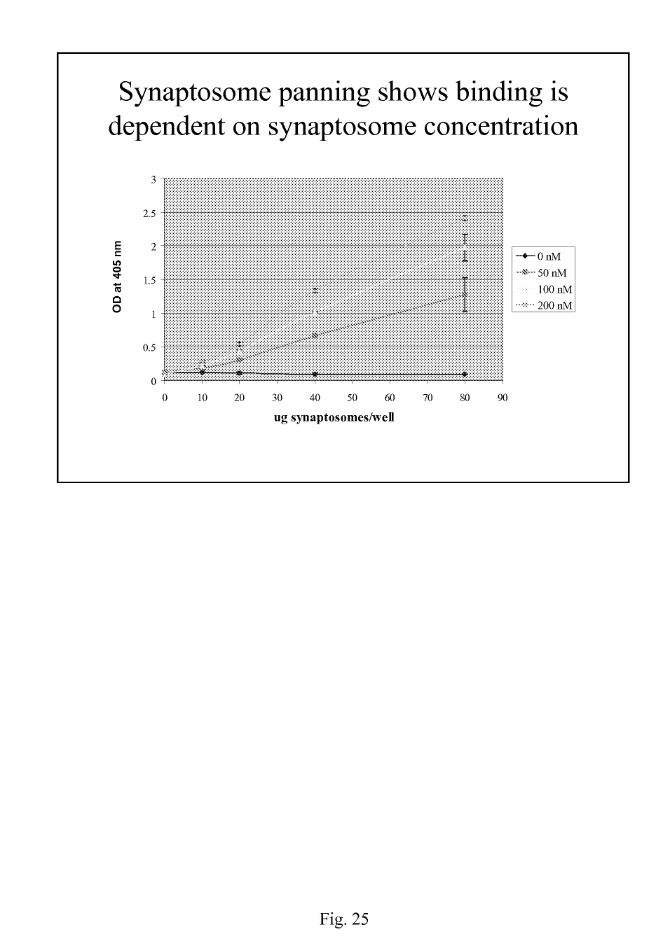

Results: As shown in FIG. 25, ADDL- and 20C2-labeled synaptosomes appeared to bind to the anti-mouse IgG-coated assay plates. None of the synaptosome controls showed any signal. 20C2 showed good linear binding to the anti-mouse IgG-coated assay plates. There was no saturation as might be expected at 80 mg/well. Statistics: Data is shown as the mean of duplicate values with error bars representing SEM.

Established: These results further support other information disclosed herein.

### EXAMPLE 11

### ADDL—Synaptosome Binding

### Using Cholera Toxin Subunit B to Immobilize Synaptosomes Shows ADDL and Synaptosome Concentration Dependent Binding

Parameters: Assay plate wells were coated with cholera toxin subunit B.

Synaptosomes were bound and visualized using ADDLs and 20C2 antibody.

Rationale: Prior procedures involve significant processing of synaptosomes, which causes synaptosome loss and is time consuming. Since CT-B binds to lipid rafts, this can be an alternative method to immobilize synaptosomes to assay wells.

Action: Cholera toxin subunit B (CT-B, Sigma), was diluted to 10 mg/ml with TBS (20 mM Tris-HCl, pH 7.5, 0.8% NaCl) and 100 ml/well (1 mg) allowed to bind to Immulon 4 Removawell strips (Dynatech Labs) overnight in the cold room. Unbound sites were blocked with 3×200 ml 2% BSA in TBS (20 mM Tris-HCl, pH 7.5, 0.8% NaCl)×10 min at RT. Synaptosomes were centrifuged at 5,000 g×5 min at 4° C. and washed with 2×1 ml of BSA/F12 and resuspended in BSA/F12. Synaptosomes were diluted to the appropriate volumes, 0, 10, 20, 40, and 80 mg/well were added to the wells, and synaptosomes were allowed to bind at 4° C. for 1 hr. Synaptosomes are washed with 3×200 ml of BSA/F12. ADDLs were diluted (10 nM, 50 nM, and 100 nM), added to wells, and allowed to bind for 1 hr at 37° C. The samples were washed as above with BSA/F12. Monoclonal 20C2 IgG (1.52 mg/ml) was diluted 1:1000 in BSA/F12, and 100 ml/well added to the prepared assay plate. The plate was incubated for 2 hr at 4° C. The samples were washed as above with BSA/F12. HRP-linked anti-mouse IgG (Amersham) was diluted 1:2000 in BSA/TBS and incubated 100 ml/well for 1 hr at RT. Following washing with BSA/TBS as above and rinsing 3× under running dH₂O, binding was visualized using 100 ml/well of Bio-Rad peroxidase substrate. Color was developed at RT and read at 405 nm on a Dynex MRX Microplate Reader. Statistics: Data is shown as the mean of duplicate values with error bars representing SEM.

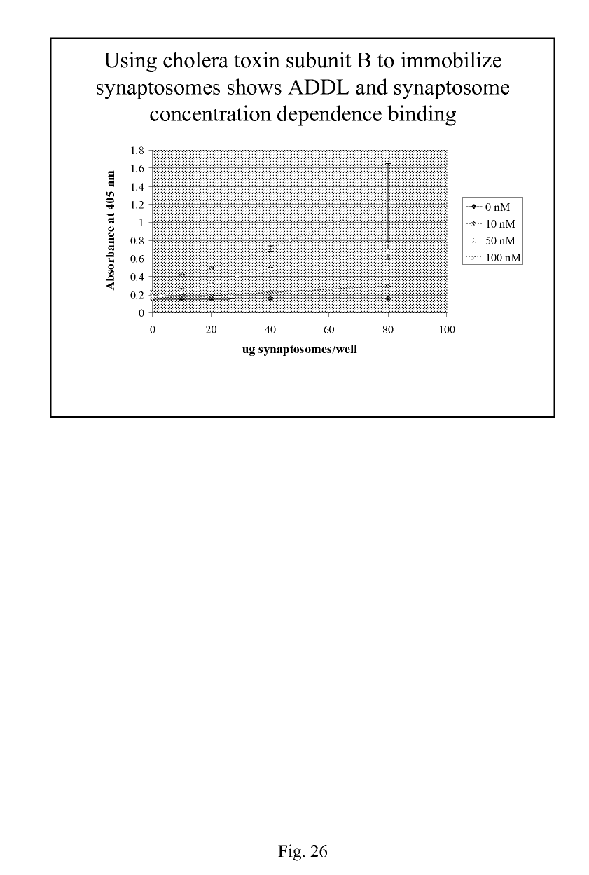

Results: As shown in FIG. 26, absorbance is dependent on ADDL concentration and on synaptosome concentration. Duplicate wells displayed good reproducibility except for one data point.

Established: CT-B can be used to immobilize synaptosomes.

### EXAMPLE 12

### ADDL—Synaptosome Immunoprecipitation

Immunoprecipitation of ADDL-Treated Synaptosomes Using Magnetic Beads Coated with an Anti-ADDL Monoclonal Antibody (20C2)

Synaptosomes were incubated with ADDLs or vehicle in F12/FBS (F12 media, 5% FBS). Treated-synaptosomes were immunoprecipitated using magnetic beads coated with an anti-ADDL monoclonal antibody (Dyna-20C2) in F12/FBS. The presence of synaptic markers was assessed in different fractions using an anti-PSD95 antibody in standard Western blots.

Parameter: Immunoprecipitate ADDL-treated synaptosomes using Dyna-20C2.

Reason: Previous information generated using the M71/2 antibody specific for ADDLs was confirmed using an anti-ADDL monoclonal antibody (20C2). Additionally, 20C2 recognizes high molecular weight ADDLs, which are bioactive. Thus, 20C2 would be expected to recognize ADDL binding to synaptosomes given other information disclosed herein.

Actions: Incubation of ADDLs with synaptosomes: Synaptosomes were prepared according to standard protocols. 75 ug synaptosomes were incubated with 300 nM ADDLs (2.5 ul ADDLs Jan. 10, 2005) or vehicle in 500 ul F12/FBS (F12 media, 5% FBS) for 3 hours at 4 C with rotation. To remove ADDLs in solution, samples were centrifuged at 5,000 g for 10 min at 4 C, and washed 3×5 min with 1 ml F12/FBS. Supernatants were stored at 4 C. Immunoprecipitation using Dyna-20C2: Dynabeads M-500 subcellular was coated with 20C2 according to procedures provided by the manufacturer. Treated synaptosomes were resuspend in 300 ul F12/FBS. 0.250 mg Dyna-M71.2 was washed in PBS, and added to synaptosomes, and they were incubated overnight at 4° C. with rotation. Beads were recovered with magnet. Beads were washed 9×12 min with 1 ml F12/FBS, and 2×12 min with 1 ml F12. Supernatants were stored as “Unbound” and “Washes”. Pellet (“Bound”) was dissolved in 50 ul SLB. “Unbound” “W1” and “W2” were centrifuged to 20,000 g for 20 min, and their pellets were dissolved in 60 ul SLB. Immunoblotting: 15 ul of each sample were loaded in an 4-20% Tris-Glycine Gel. Gel was run at 180 V for 45 min, and transferred to a nitrocellulose membrane at 100 V at 4 C for 1 h. Membrane was blocked with 5% milk in TBS-T for 1 hour at RT and incubated with PSD95 antibody for 1 hour at RT. PSD95 (MA1-045 from ABR): 1:4,000 Mouse-HRP: 1:50,000. Membrane was washed 3×10 min in TBS-T, and incubated with anti-mouse IgG-HRP for 1 hour at RT. After wash 3×10 min with TBS-T, gel was developed with enhanced chemiluminescence (ECL).

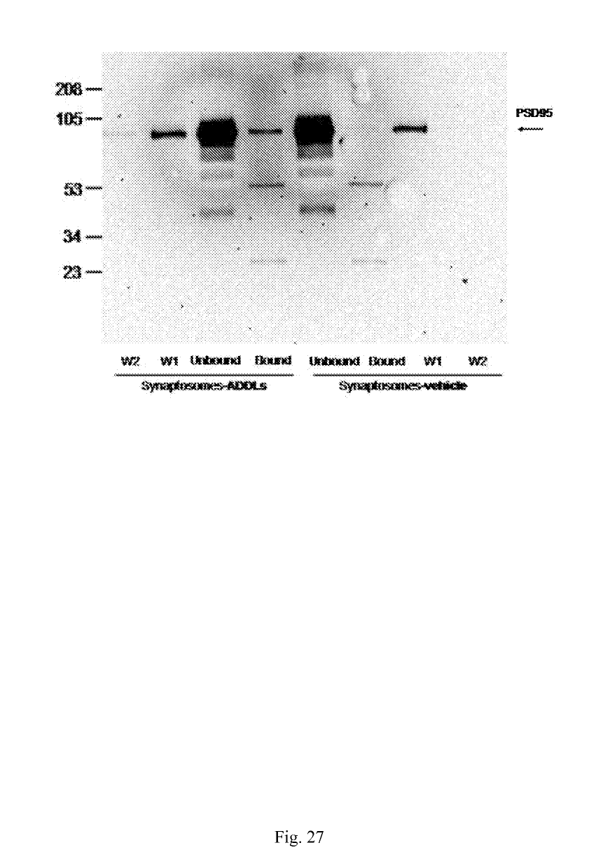

Results: As shown in FIG. 27, PSD95 can be detected in the Bound fraction of ADDL-synaptosomes, but not in vehicle-synaptosomes.

Established: ADDLs-synaptosomes can be immunoprecipitated using 20C2.

### EXAMPLE 13

### ADDL Binding to Cortical PSDs

Parameters: Assess the binding of ADDLs to isolated cortical PSDs and/or AZs

Rationale: ADDLs appeat to bind to PSDs and not to Actives Zones, in experiments of Far Western Blot and Ant2.041: Isolated PSDs or AZ incubated with ADDLs and filtered with YM-100.

Actions: Fractionation of synaptosomes: Cortical synaptosomes were prepared according to standard procedures, with minor modifications (see e.g. Phillips et al. Neuron, vol. 32, pp. 63-77). 900 ug synaptosomes were diluted in 5 ml 0.1 mM CaCl2, and osmotic lysis was performed for 30 min. The mix was brought to 20 mM Tris pH:6 and 1% TX-100 (with 5 ml solution 2×) and membranes were solubilizated for 30 min in ice. The insoluble material (Synaptic Junctions) was pelleted by centrifugation 30,000 g 45 min. Synaptic junctions (SJs) were resuspended in 3.5 ml 20 mM Tris pH 8.8 and 1% TX100 (Triton X-100). After incubation overnight at 4° C., sample was centrifuged 40,000 g for 45 min (25 k rpm in TLA 1300 rotor). Pellet contained PSD. Supernatant was dialyzed against 0.1 mM CaCl2, 20 mM Tris pH:6 and 1% TX-100 (3×10 hours), and centrifuged 40,000 g for 45 min as above. This pellet contained Active Zones (AZ). AZ and PSD were resuspended in 30 ul TBS with protease inhibitors. Samples were briefly sonicated and protein concentration was obtained using BSA assay: 3 ug/ul for both samples. ELISA: ELISA was performed following standard protocols, with minor modifications: Wells were coated with 0.25, 0, 5, 1, 2.5 or 5 ug of sample were dissolved in 100 ul TBS+2% BSA (TBS/BSA), overnight at 4 C. Plate was blocked with 200 ul TBS/BSA 3×20 min at RT. 100 nM ADDLs was added to each well in 100 ul TBS-T/BSA, and incubated for 2 hours at RT. Plate was washed 3×10 min with TBS-T at RT. For detection, M71/2 polyclonal Ab (ADDL specific) was used at 1:1,000 in 100 ul TBS-T/BSA. Incubate for 1 hour at RT and wash 3×10 min with 200 ul TBS-T at RT. Rabbit-HRP (Amersham) 1:2,000 was used as secondary Ab 100 ul TBS-T. Incubate for 1 hour at RT and wash 3×10 min with 200 ul TBS-T. 100 ul freshly prepared HRP substrate (Bio-Rad “Peroxidase substrate Kit”. 172-1064) were added and color was developed for 45 min at RT. Color was measure at 405 nm. Native Western Blot (WB): 9 ug of PSDs or Active Zones were dissolved in 15 ul Native Sample Buffer and loaded in a Tris-Glycine 4-20% Gel. Without beta-mercaptoethanol, without boiling the samples, and without SDS in the running buffer. The gel was run at 100 V and transferred to nitrocellulose membrane for 1.5 h. at 120 V at 4 C. Following trnasfer, the membrane was blocked with 5% milk in TBS-T 1 hour at RT and incubated with primary antibody (Ab) overnight at 4 C. PSD95 (MA1-045 from ABR-Affinity BioReagents-): 1:4,000, Mouse-HRP: 1:50,000. Syntaxin (MAB336 from CHEMICON): 1:4,000, Mouse-HRP: 1:10,000. Washed 3×10 min in TBS-T, and incubated with anti-mouse IgG-HRP for 1 hour at RT. After wash 3×10 min with TBS-T, gel was developed with ECL.

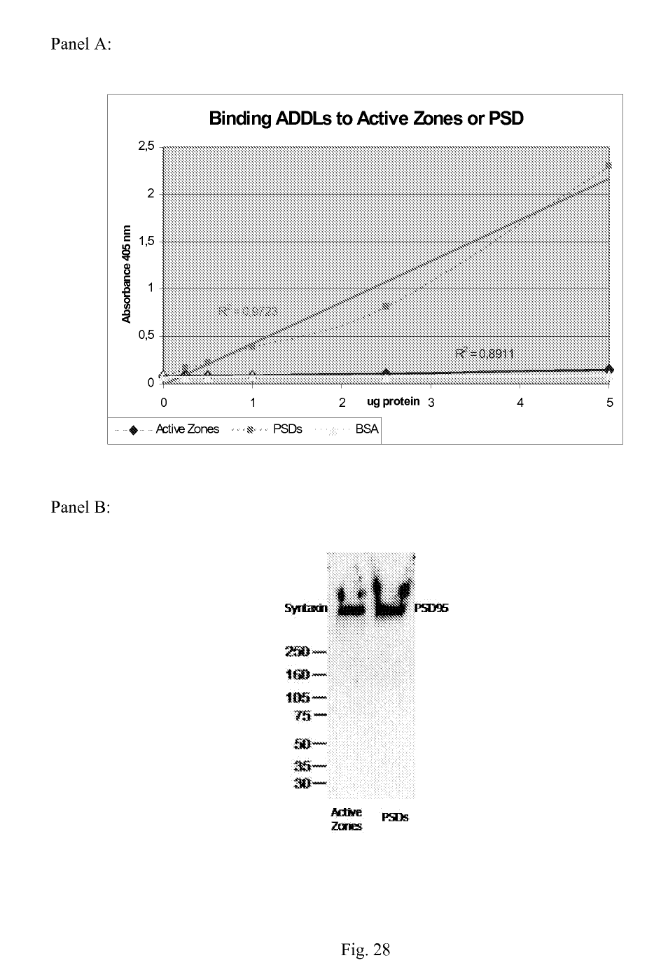

Result: As shown in FIG. 28, Panels A & B, ADDLs only bind to PSDs and not to AZs. Both, Active Zones and PSDs, remain as multiprotein complexes after the preparation described herein (i.e., sonication, etc.). Thus, they remain in the well in a native gel electrophoresis and are unable to enter the gel matrix.

### EXAMPLE 14

### Formation of Biotin-Labeled ADDLS for Use in Biochemical and Cell Biological Measurements

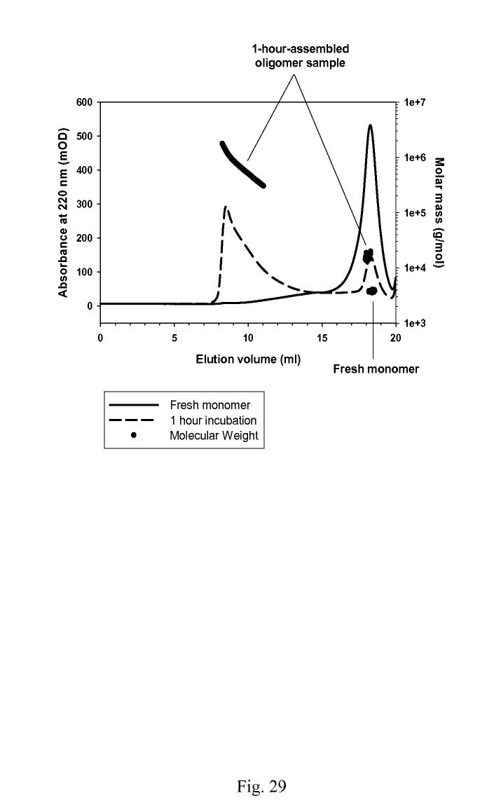

As shown in FIG. 29, the incorporation of biotin into ADDLs allows for the production of LMW and HMW oligomers. Biotin-Abeta(1-42) will allow for the direct detection of ADDLs using streptavidin-linked reagents.

Referring to FIG. 29, Biotin-ADDLs (i.e., b-ADDLs, B-ADDLs, and or BADDLs) oligomerize into trimer/tetramer and HMW assemblies. When used in a 1:4 ratio with native Abeta(1-42), Biotin-Abeta(1-42) allows for the correct profile of ADDL assembly (see e.g., U.S. Pat. No. 6,218,506; and the like). A one hour incubation of 100 uM total peptide (20 uM Biotinylated, 80 uM native) in 1×PBS (w/o Ca and Mg) at 37 C leads to significant soluble oligomer formation, compared to the fresh peptide monomer dilution at time zero. 1 ml samples were produced using standard ADDL preparation methodologies, but using PBS as diluent, after HFIP evaporation and DMSO resuspension, instead of F-12 tissue culture medium. The solid curves in the figure represent the absorbance of peptide and peptide assemblies at 220 nm. These curves were obtained by monitoring the absorbance of 300 ul samples injected onto a Superdex-200 HR 10/30 column at a flow rate of 0.5 ml/min in 1×PBS (w/o Ca and Mg) at room temperature. An AKTA Basic chromatography system, using Unicorn software, operated the system and collected the data. The dotted curves represent the molecular weight (MW) values determined by Multi-Angle Laser Light Scattering (MALLS). A Wyatt Technologies DAWN EOS MALLS instrument was connected inline with the HPLC column and absorbance flow cell, and the Optilab rEX instrument was used to determine the protein concentration of eluting species. Using Wyatt Technologies' ASTRA V software, the MW profiles were recorded and fitted. As can be seen in the time zero fresh monomer sample, a MW corresponding to monomer is observed in the second peak, eluting at roughly 20 19 min. The one hour sample has significantly oligomerized. The first peak, which trails significantly from 8 ml to 15 ml, contains species from the million Dalton range to the low hundred thousands of Dalton. The second peak, rather than containing predominantly monomeric peptide, now contains species in the trimer & tetramer range. While the monomer MW is roughly 4800 Da, 1 hour sample contains low molecular weight (LMW) oligomers in the 15000 to 20000 range, indicating stable formation of these species.

Fluorescein labeled ADDLs assemble similarly to biotin labeled ADDLs (data not shown).

### EXAMPLE 15

### Characterization of ADDLs Labeled with Biotin

Parameters: ADDLs from a mixture of biotinylated and unlabeled Abl-42 were fractionated by SEC and analyzed by native and SDS-PAGE Western blots, probed for the biotin label or with monoclonal 6E10 and 20C2 antibodies.

Rationale: Biotinylated ADDLs provide another tool, independent of antibodies, for research. It is necessary to analyze ADDLs produced with biotinylated Abl-42 to see if the biotin label affects assembly, structure or function of the oligomers.

Action: ADDLs were prepared from a mixture (1:4.7 mol:mol) of biotinylated and unlabeled Ab 1-42 by mixing HFIP solutions of the two peptides and air drying overnight followed by drying on a Savant Speed-Vac dryer. The HFIP film was dissolved in DMSO to ˜5 mM and diluted with ice cold F12 to ˜100 μM, vortexed briefly and allowed to sit at 4° C. overnight. The sample was centrifuged at 14,000 g×10 min at 4° C. and transferred to a clean tube. Protein concentration was determined by Coomassie Plus protein assay (Pierce) using a BSA standard. Biotinylated ADDLs were subjected to SEC on a Superdex 75 HR/10/30 column and the fractions analyzed by dot blot for distribution of the biotin label. Biotinylated ADDLs and SEC fractions were diluted with F12 and native sample buffer (final concentration of 5 mM Tris-HCl, pH 6.8, 38.3 mM glycine, 10% glycerol, 0.017% bromphenol blue) or Tricine sample buffer (Bio-Rad) and analyzed (˜60 pmoles for silver stain or ˜20 pmoles for Western blot) by PAGE. Unlabeled ADDLs were run for comparison. The native gel (10% T acrylamide, 5% C resolving gel) used a running buffer of 5 mM Tris, 38.4 mM glycine, pH 8.3 (Betts et al. (1999) Meth. Enzymol., vol. 309, pp. 333-350) at 120V, 4° C. for 3 hr. The SDS gel (10-20% Tris-Tricine precast gel, Bio-Rad) was run with Tris/glycine/SDS buffer (Bio-Rad) at 120V for 80 min at RT. Silver stain was performed with a SilverXpress silver stain kit (Invitrogen) using the Tricine gel protocol. Alternatively, the gels were electroblotted onto Hybond ECL nitrocellulose using 25 mM Tris-192 mM glycine, 20% v/v methanol, pH 8.3 at 100 V for 1 hr at 4°. The blots were blocked with 5% milk in TBS-T (0.1% Tween-20 in 20 mM Tris-HCl, pH 7.5, 0.8% NaCl) for 1 hr at RT.

Biotin probe: An avidin-biotinylated HRP complex (Vectastain ABC standard kit; Vector Labs) was formed by diluting the A and B reagents 1:500 in 5% milk/TBS-T and pre-incubating for 30 min at RT. The blots were incubated with the preformed complex for 1 hr and washed 3×10 min with TBS-T, rinsed 2× with dH₂O, developed with SuperSignal West Femto Maximum Sensitivity substrate (Pierce; 1:1 dilution with ddH₂O) and read on a Kodak Image Station.

Immunostain: Monoclonal anti-Ab (6E10, Signet) or anti-ADDLs (20C2; M. Lambert; IgG PVO2-109, 1.52 mg/ml) were diluted 1:1000 in milk/TBS and incubated with the blots for 90 min at RT. Following washing 3×10 min with TBS-T, the blots were incubated with HRP-linked anti-mouse Ig (1:40,000 in milk/TBST; Amersham) for 90 min at RT. The blots were washed 3×10 min with TBS-T, rinsed 2× with dH₂O, developed with SuperSignal West Femto Maximum Sensitivity substrate (Pierce; 1:1 dilution with ddH₂O) and read on a Kodak Image Station.

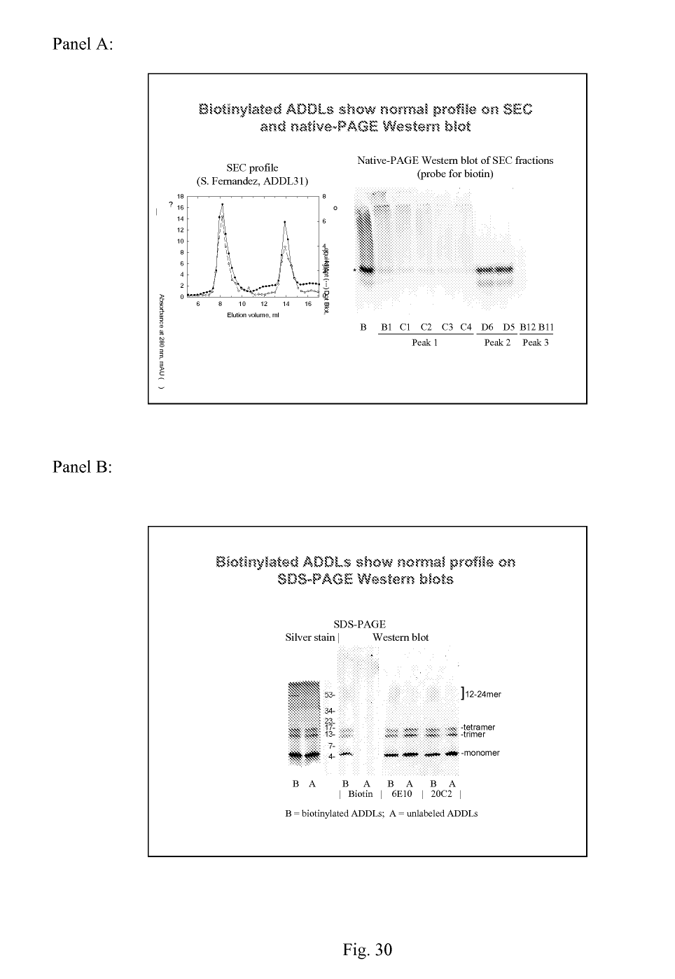

Results: Biotinylated ADDLs have a SEC profile (FIG. 30, top left panel) similar to that previously observed using unlabeled ADDLs. The dot blot for the biotin label shows a similar profile to the absorbance readings at 280 nm. The native-PAGE Western blot of SEC fractions using a probe for the biotin label (FIG. 30, top right panel) shows slower moving oligomers in Peak 1. Most of the major native species (*), as well as a faster moving band, were in Peak 2. There was no staining in Peak 3 fractions. Silver stain of biotinylated ADDLs following SDS-PAGE showed a similar pattern to unlabeled ADDLs (FIG. 30, bottom left panel). There was a single minor band at ˜52 kDa in the biotinylated ADDLs. Western blot following SDS-PAGE of biotinylated and unlabeled ADDLs (FIG. 30, bottom right panel) showed specificity of the probe for biotin. Both 6E10 and 20C2 showed similar immunostaining patterns for biotinylated and unlabeled ADDLs. The ˜52 kDa band in silver stain does not appear in any of the Western blots.

Established: The mixture of biotinylated and unlabeled Abl-42 forms ADDLs with typical electrophoretic profiles on both native and SDS gels. By probing for the biotin label, distribution of the various oligomeric species can be detected independent of the epitope-specific immunostaining obtained with antibodies. Biotinylated ADDLs also fractionate on size exclusion chromatography (SEC) in a similar pattern as unlabeled ADDLs.

### REFERENCES

Allison D W, Chervin A S, Gelfand V I, Craig A M (2000) Postsynaptic scaffolds of excitatory and inhibitory synapses in hippocampal neurons: maintenance of core components independent of actin filaments and microtubules. J Neurosci 20: 4545-4554.
Boeckers, T. M., M. G. Mameza, M. R. Kreutz, J. Bockmann, C. Weise, F. Buck, D. Richter, E. D. Gundelfinger, and H. J. Kreienkamp. 2001. Synaptic scaffolding proteins in rat brain. Ankyrin repeats of the multidomain Shank protein family interact with the cytoskeletal protein alpha-fodrin. J. Biol. Chem. 276:40104-40112.
Buttini M, Yu G Q, Shockley K, Huang Y, Jones B, Masliah E, Mallory M, Yeo T, Longo F M, Mucke L (2002) Modulation of Alzheimer-like synaptic and cholinergic deficits in transgenic mice by human apolipoprotein E depends on isoform, aging, and overexpression of amyloid beta peptides but not on plaque formation. J Neurosci 22: 10539-10548.
Caughey B, Lansbury P T (2003) Protofibrils, pores, fibrils, and neurodegeneration: separating the responsible protein aggregates from the innocent bystanders. Annu Rev Neurosci 26: 267-298.
Chang L, Bakhos L, Wang Z, Venton D L, Klein W L (2003) Femtomole immunodetection of synthetic and endogenous amyloid-beta oligomers and its application to Alzheimer's disease drug candidate screening. J Mol Neurosci 20: 305-313.
Chin J, Palop J J, Yu G Q, Kojima N, Masliah E, Mucke L (2004) Fyn kinase modulates synaptotoxicity, but not aberrant sprouting, in human amyloid precursor protein transgenic mice. J Neurosci 24: 4692-4697.
Chromy B A, Nowak R J, Lambert M P, Viola K L, Chang L, Velasco P T, Jones B W, Fernandez S J, Lacor P N, Horowitz P, Finch C E, Krafft G A, Klein W L (2003) Self-assembly of Abeta(1-42) into globular neurotoxins. Biochemistry 42: 12749-12760.
Cirelli C, Tononi G (2000) Differential expression of plasticity-related genes in waking and sleep and their regulation by the noradrenergic system. J Neurosci 20: 9187-9194.
Coyle J T (1987) Alzheimer's Disease. In: Encyclopedia of Neuroscience (Adelman G, ed), pp 29-31. Boston-Basel-Stuttgart: Birkhäuser.
Crick F (1982) Do dendritic spines twitch? Trends Neurosci 5: 44-46.
Dickey C A, Loring J F, Montgomery J, Gordon M N, Eastman P S, Morgan D (2003) Selectively reduced expression of synaptic plasticity-related genes in amyloid precursor protein+presenilin-1 transgenic mice. J Neurosci 23: 5219-5226.
Dodart J C, Bales K R, Gannon K S, Greene S J, DeMattos R B, Mathis C, DeLong C A, Wu S, Wu X, Holtzman D M, Paul S M (2002) Immunization reverses memory deficits without reducing brain Abeta burden in Alzheimer's disease model. Nat Neurosci 5: 452-457.
Fiala J C, Spacek J, Harris K M (2002) Dendritic spine pathology: cause or consequence of neurological disorders? Brain Res Brain Res Rev 39: 29-54.
Fong D K, Rao A, Crump F T, Craig A M (2002) Rapid synaptic remodeling by protein kinase C: reciprocal translocation of NMDA receptors and calcium/calmodulin-dependent kinase II. J Neurosci 22: 2153-2164.
Freeman W M, Brebner K, Lynch W J, Patel K M, Robertson D J, Roberts D C, Vrana K E (2002) Changes in rat frontal cortex gene expression following chronic cocaine. Brain Res Mol Brain Res 104: 11-20.
Fujimoto T, Tanaka H, Kumamaru E, Okamura K, Miki N (2004) Arc interacts with microtubules/microtubule-associated protein 2 and attenuates microtubule-associated protein 2 immunoreactivity in the dendrites. J Neurosci Res 76: 51-63.
Gibson W W, Eckert G P, Igbavboa U, Muller W E (2003) Amyloid beta-protein interactions with membranes and cholesterol: causes or casualties of Alzheimer's disease. Biochim Biophys Acta 1610: 281-290.
Gong Y, Chang L, Viola K L, Lacor P N, Lambert M P, Finch C E, Krafft G A, Klein W L (2003) Alzheimer's disease-affected brain: Presence of oligomeric A{beta} ligands (ADDLs) suggests a molecular basis for reversible memory loss. Proc Natl Acad Sci USA 100: 10417-10422.
Gouras G K, Tsai J, Naslund J, Vincent B, Edgar M, Checker F, Greenfield J P, Haroutunian V, Buxbaum J D, Xu H, Greengard P, Relkin N R (2000) Intraneuronal Abeta42 accumulation in human brain. Am J Pathol 156: 15-20.
Guzowski J F (2002) Insights into immediate-early gene function in hippocampal memory consolidation using antisense oligonucleotide and fluorescent imaging approaches. Hippocampus 12: 86-104.
Guzowski J F, Lyford G L, Stevenson G D, Houston F P, McGaugh J L, Worley P F, Barnes C A (2000) Inhibition of activity-dependent arc protein expression in the rat hippocampus impairs the maintenance of long-term potentiation and the consolidation of long-term memory. J Neurosci 20: 3993-4001.
Hainfellner J A, Liberski P P, Guiroy D C, Cervenakova L, Brown P, Gajdusek D C, Budka H (1997) Pathology and immunocytochemistry of a kuru brain. Brain Pathol 7: 547-553.
Hardy J, Selkoe D J (2002) The amyloid hypothesis of Alzheimer's disease: progress and problems on the road to therapeutics. Science 297: 353-356.
Hock C, Konietzko U, Streffer J R, Tracy J, Signorell A, Muller-Tillmanns B. Lemke U, Henke K, Moritz E, Garcia E, Wollmer A, Umbricht D, de Quervain D J F, Hofmann M, Maddalena A, Papassotiropoulos A, Nitsch R M (2003) Antibodies against b-amyloid slow cognitive decline in Alzheimer's disease. Neuron 38: 547-554.
Hsia A Y, Masliah E, McConlogue L, Yu G Q, Tatsuno G, Hu K, Kholodenko D, Malenka R C, Nicoll R A, Mucke L (1999) Plaque-independent disruption of neural circuits in Alzheimer's disease mouse models. Proc Natl Acad Sci USA 96: 3228-3233.
Inagaki N, Nishizawa M, Arimura N, Yamamoto H, Takeuchi Y, Miyamoto E, Kaibuchi K, Inagaki M (2000) Activation of Ca2+/calmodulin-dependent protein kinase II within post-synaptic dendritic spines of cultured hippocampal neurons. J Biol Chem 275: 27165-27171.
Katzman R, Terry R, DeTeresa R, Brown T, Davies P, Fuld P, Renbing X, Peck A (1988) Clinical, pathological, and neurochemical changes in dementia: a subgroup with preserved mental status and numerous neocortical plaques. Ann Neurol 23: 138-144.
Kaufmann W E, Moser H W (2000) Dendritic anomalies in disorders associated with mental retardation. Cereb Cortex 10: 981-991.
Kayed R, Head E, Thompson J L, McIntire T M, Milton S C, Cotman C W, Glabe C G (2003) Common structure of soluble amyloid oligomers implies common mechanism of pathogenesis. Science 300: 486-489.
Kelly M P, Deadwyler S A (2003) Experience-dependent regulation of the immediate-early gene arc differs across brain regions. J Neurosci 23: 6443-6451.
Kirkitadze M D, Bitan G, Teplow D B (2002) Paradigm shifts in Alzheimer's disease and other neurodegenerative disorders: the emerging role of oligomeric assemblies. J Neurosci Res 69: 567-577.
Klein W L (2002) Ab toxicity in Alzheimer's disease: globular oligomers (ADDLs) as new vaccine and drug targets. Neurochem Int 41: 345-352.
Klein W L, Kraffi G A, Finch C E (2001) Targeting small Abeta oligomers: the solution to an Alzheimer's disease conundrum? Trends Neurosci 24: 219-224.
Kotilinek L A, Bacskai B, Westerman M, Kawarabayashi T, Younkin L, Hyman B T, Younkin S, Ashe K H (2002) Reversible memory loss in a mouse transgenic model of Alzheimer's disease. J Neurosci 22: 6331-6335.
Kovacs G G, Head M W, Hegyi I, Bunn T J, Flicker H, Hainfellner J A, McCardle L, Laszlo L, Jarius C, Ironside J W, Budka H (2002) Immunohistochemistry for the prion protein: comparison of different monoclonal antibodies in human prion disease subtypes. Brain Pathol 12: 1-11.
Lambert M P, Barlow A K, Chromy B A, Edwards C, Freed R, Liosatos M, Morgan T E, Rozovsky I, Trommer B, Viola K L, Wals P, Zhang C, Finch C E, Kraffi G A, Klein W L (1998) Diffusible, nonfibrillar ligands derived from Abeta1-42 are potent central nervous system neurotoxins. Proc Natl Acad Sci USA 95: 6448-6453.
Lambert M P, Viola K L, Chromy B A, Chang L, Morgan T E, Yu J, Venton D L, Krafft G A, Finch C E, Klein W L (2001) Vaccination with soluble Abeta oligomers generates toxicity-neutralizing antibodies. J Neurochem 79: 595-605.
Link W, Konietzko U, Kauselmann G, Krug M, Schwanke B, Frey U, Kuhl D (1995) Somatodendritic expression of an immediate early gene is regulated by synaptic activity. Proc Natl Acad Sci USA 92: 5734-5738.
Lue L F, Kuo Y M, Roher A E, Brachova L, Shen Y, Sue L, Beach T, Kurth J H, Rydel R E, Rogers J (1999) Soluble amyloid beta peptide concentration as a predictor of synaptic change in Alzheimer's disease. Am J Pathol 155: 853-862.
Lyford G L, Yamagata K, Kaufmann W E, Barnes C A, Sanders L K, Copeland N G, Gilbert D J, Jenkins N A, Lanahan A A, Worley P F (1995) Arc, a growth factor and activity-regulated gene, encodes a novel cytoskeleton-associated protein that is enriched in neuronal dendrites. Neuron 14: 433-445.
McLaurin J, Chakrabartty A (1996) Membrane disruption by Alzheimer beta-amyloid peptides mediated through specific binding to either phospholipids or gangliosides. Implications for neurotoxicity. J Biol Chem 271: 26482-26489.
Moga D E, Calhoun M E, Chowdhury A, Worley P, Morrison J H, Shapiro M L (2004) Activity-regulated cytoskeletal-associated protein is localized to recently activated excitatory synapses. Neuroscience 125: 7-11.
Mucke L, Masliah E, Yu G Q, Mallory M, Rockenstein E M, Tatsuno G, Hu K, Kholodenko D, Johnson-Wood K, McConlogue L (2000) High-level neuronal expression of abeta 1-42 in wild-type human amyloid protein precursor transgenic mice: synaptotoxicity without plaque formation. J Neurosci 20: 4050-4058.
Naisbitt, S., E. Kim, J. C. Tu, B. Xiao, C. Sala, J. Valtschanoff, R. J. Weinberg, P. F. Worley, and M. Sheng. 1999. Shank, a novel family of postsynaptic density proteins that binds to the NMDA receptor/PSD-95/GKAP complex and cortactin. Neuron. 23:569-582.
Oddo S, Caccamo A, Shepherd J D, Murphy M P, Golde T E, Kayed R, Metherate R, Mattson M P, Akbari Y, LaFerla F M (2003) Triple-transgenic model of Alzheimer's disease with plaques and tangles: intracellular Abeta and synaptic dysfunction. Neuron 39: 409-421.
Rao A, Craig A M (2000) Signaling between the actin cytoskeleton and the postsynaptic density of dendritic spines. Hippocampus 10: 527-541.
Rao A, Kim E, Sheng M, Craig A M (1998) Heterogeneity in the molecular composition of excitatory postsynaptic sites during development of hippocampal neurons in culture. JNeurosci 18: 1217-1229.
Sala, C., V. Piech, N. R. Wilson, M. Passafaro, G. Liu, and M. Sheng. 2001. Regulation of dendritic spine morphology and synaptic function by Shank and Horner. Neuron. 31:115-130.
Schenk D (2002) Amyloid-beta immunotherapy for Alzheimer's disease: the end of the beginning. Nat Rev Neurosci 3: 824-828.
Selkoe D J (2002) Alzheimer's disease is a synaptic failure. Science 298: 789-791.
Sheng M, Pak D T (1999) Glutamate receptor anchoring proteins and the molecular organization of excitatory synapses. Ann N Y Acad Sci 868: 483-493.
Sheng, M., and E. Kim. 2000. The Shank family of scaffold proteins. J. Cell Sci. 113:1851-1856.
Steward O, Worley P F (2001) A cellular mechanism for targeting newly synthesized mRNAs to synaptic sites on dendrites. Proc Natl Acad Sci USA 98: 7062-7068.
Sweatt J D (2001) The neuronal MAP kinase cascade: a biochemical signal integration system subserving synaptic plasticity and memory. J Neurochem 76: 1-10.
Terry R D, Masliah E, Salmon D P, Butters N, DeTeresa R, Hill R, Hansen L A, Katzman R (1991) Physical basis of cognitive alterations in Alzheimer's disease: synapse loss is the major correlate of cognitive impairment. Ann Neurol 30: 572-580.
Tong L, Thornton P L, Balazs R, Cotman C W (2001) Beta-amyloid-(1-42) impairs activity-dependent cAMP-response element-binding protein signaling in neurons at concentrations in which cell survival Is not compromised. J Biol Chem 276: 17301-17306.
Tu, J. C., B. Xiao, S, Naisbitt, J. P. Yuan, R. S. Petralia, P. Brakeman, A. Doan, V. K. Aakalu, A. A. Lanahan, M. Sheng, and P. F. Worley. 1999. Coupling of mGluR/Horner and PSD-95 complexes by the Shank family of postsynaptic density proteins. Neuron. 23:583-592.
Van Dam D, D'Hooge R, Staufenbiel M, Van Ginneken C, Van Meir F, De Deyn P P (2003) Age-dependent cognitive decline in the APP23 model precedes amyloid deposition. Eur J Neurosci 17: 388-396.
Verdier Y, Zarandi M, Penke B (2004) Amyloid beta-peptide interactions with neuronal and glial cell plasma membrane: binding sites and implications for Alzheimer's disease. J Pept Sci 10: 229-248.
Walsh D M, Klyubin I, Fadeeva J V, Cullen W K, Anwyl R, Wolfe M S, Rowan M J, Selkoe D J (2002) Naturally secreted oligomers of amyloid beta protein potently inhibit hippocampal long-term potentiation in vivo. Nature 416: 535-539.
Walsh D M, Selkoe D J (2004) Oligomers on the brain: the emerging role of soluble protein aggregates in neurodegeneration. Protein Pept Lett 11: 213-228.
Wang H W, Pasternak J F, Kuo H, Ristic H, Lambert M P, Chromy B, Viola K L, Klein W L, Stine W B, Kraffi G A, Trommer B L (2002) Soluble oligomers of beta amyloid (1-42) inhibit long-term potentiation but not long-term depression in rat dentate gyrus. Brain Res 924: 133-140.
Wang Q, Walsh D M, Rowan M J, Selkoe D J, Anwyl R., Block of long-term potentiation by naturally secreted and synthetic amyloid beta-peptide in hippocampal slices is mediated via activation of the kinases c-Jun N-terminal kinase, cyclin-dependent kinase 5, and p38 mitogen-activated protein kinase as well as metabotropic glutamate receptor type 5. J. Neurosci. 2004 March 31; 24(13):3370-8.
Wang Z, Chang L, Klein W L, Thatcher G R, Venton D L (2004) Per-6-substituted-per-6-deoxy beta-cyclodextrins inhibit the formation of beta-amyloid peptide derived soluble oligomers. J Med Chem 47: 3329-3333.
Weiner H L, Selkoe D J (2002) Inflammation and therapeutic vaccination in CNS diseases. Nature 420: 879-884.
Yuste R, Bonhoeffer T (2001) Morphological changes in dendritic spines associated with long-term synaptic plasticity. Annu Rev Neurosci 24: 1071-1089.

The patents, patent applications, as well as any other scientific and technical writings referred to in this document are incorporated by reference to the extent that they are not contradictory.

The foregoing disclosure of preferred embodiments of the invention is presented for purposes of illustration and description. It is not intended to be exhaustive or to limit the invention to the precise form or forms disclosed. The description was selected to best explain the principles of the invention and practical application of these principles to enable others skilled in the art to best practice the invention in various embodiments and various modifications as are suited to the particular use contemplated. The scope of the invention is not to be limited by the specification.

1-114. (canceled)

115: A method for treating an ADDL-related disease comprising administering to subject in need of treatment one or more compounds that antagonize the binding of ADDLs to one or more receptors, wherein the one or more receptors is selected from the group consisting of synGAP, proSAP2/Shank3, a metabotropic glutamate receptor, a kainate sub-type glutamate receptor, an AMPA sub-type glutamate receptor, a NMDA sub-type glutamate receptor, an integrin receptor, an adhesion receptor, a trophic factor receptor, a Trk receptor, erbB4, a trophin receptor, insulin receptor, insulin growth factor receptor, a GABA receptor, a sodium/potassium ATPase, CAM kinase II, PrP protein, a receptor protein tyrosine phosphatase alpha (RPTPα) protein, a somatostatin receptor, a cannabinoid receptor, a sigma receptor, and VIP/PACAL receptor.

116. The method of claim 115, wherein the ADDL-related disease is Alzheimer's disease (AD).

117. The method of claim 115, wherein the ADDL-related disease is mild cognitive impairment (MCI).

118. The method of claim 115, wherein the ADDL-related disease is Down's syndrome.

119. The method of claim 115, wherein the one or more compounds is CNQX or a pharmaceutically acceptable derivative of CNQX.

120. A method of screening for compounds which interfere with the binding of ADDLs to one or more receptors comprising:
a) contacting tissue culture cells with ADDLs and one or more test compounds; and
b) determining the effect of the one or more test compounds on the binding of the ADDLs to one or more receptors, wherein the one or more receptors is selected from the group consisting of synGAP, proSAP2/Shank3, a metabotropic glutamate receptor, a kainate sub-type glutamate receptor, an AMPA sub-type glutamate receptor, a NMDA sub-type glutamate receptor, an integrin receptor, an adhesion receptor, a trophic factor receptor, a Trk receptor, erbB4, a trophin receptor, insulin receptor, insulin growth factor receptor, a GABA receptor, a sodium/potassium ATPase, CAM kinase II, PrP protein, a receptor protein tyrosine phosphatase alpha (RPTPα) protein, a somatostatin receptor, a cannabinoid receptor, a sigma receptor, and VIP/PACAL receptor.

121. The method of claim 120, wherein the ADDLs are labeled.

122. The method of claim 120, wherein the step of determining the effect of the one or more test compounds is carried out using an antibody that recognizes ADDLs when bound to one or more of the receptors.

123. A method of identifying compounds which interfere with the binding of ADDL surrogates to one or more receptors comprising:
a) contacting tissue culture cells with ADDL surrogates and one or more test compounds; and
b) determining the effect of the one or more test compounds on the binding of the ADDL surrogates to one or more receptors so that compounds which interfere with the binding of ADDL surrogates to one or more receptors are identified.

124. The method of claim 123, wherein the effect of one or more test compounds on the binding of the ADDL surrogates to one or more receptors is carried out by:
i) measuring the amount of arc protein that is produced using an anti-arc antibody; or
ii) measuring the punctate binding that is characteristic of ADDL binding.


SEQUENCE LISTING (59 sequences)

SEQ ID NO: 1 - protein, 11 aa, Artificial sequence
  Synthetic peptide
XXXXXXXXXXX

SEQ ID NO: 2 - protein, 36 aa, Artificial sequence
  Synthetic peptide
DSGYEVXXQKLVFFAEDVGSNKGAIIGLMVGGAIVV

SEQ ID NO: 3 - protein, 37 aa, Artificial sequence
  Synthetic peptide
DSGYEVXXQKLVFFAEDVGSNKGAIIGLMVGGXAIVV

SEQ ID NO: 4 - protein, 31 aa, Artificial sequence
  Synthetic peptide
XXXQKLVFFAEDVGSNKGAIIGLMVGGAIVV

SEQ ID NO: 5 - protein, 32 aa, Artificial sequence
  Synthetic peptide
XXXQKLVFFAEDVGSNKGAIIGLMVGGXAIVV

SEQ ID NO: 6 - protein, 31 aa, Homo sapiens
FEGYIDLGRELSTLHALLWEVLPQLSKEALL

SEQ ID NO: 7 - protein, 32 aa, Homo sapiens
YEGYCVDLATEIAKHCGFKYKLTIVGDGKYGA

SEQ ID NO: 8 - protein, 32 aa, Homo sapiens
FEGYCLDLLKELSNILGFIYDVKLVPDGKYGA

SEQ ID NO: 9 - protein, 32 aa, Homo sapiens
FEGYCIDLLRELSTILGFTYEIRLVEDGKYGA

SEQ ID NO: 10 - protein, 27 aa, Artificial sequence
  Synthetic peptide
FEGYCIDLRELSTGFYEPQLVDGKYGA

SEQ ID NO: 11 - protein, 32 aa, Homo sapiens; identical to SEQ ID NO: 8

SEQ ID NO: 12 - protein, 31 aa, Artificial sequence
  Synthetic peptide
FEGYCIDLLRELSTILGFYEVPQLVDGKYGA

SEQ ID NO: 13 - protein, 883 aa, Homo sapiens
MQKIMHISVLLSPVLWGLIFGVSSNSIQIGGLFPRGADQEYSAFRVGMVQFSTSEFRLTPHIDNLEVANSFAVTNAFCSQFSRGVYAIFGFYDKKSVNTITSFCGTLHVSFITPSFPTDGTHPFVIQMRPDLKGALLSLIEYYQWDKFAYLYDSDRGLSTLQAVLDSAAEKKWQVTAINVGNINNDKKDEMYRSLFQDLELKKERRVILDCERDKVNDIVDQVITIGKHVKGYHYIIANLGFTDGDLLKIQFGGANVSGFQIVDYDDSLVSKFIERWSTLEEKEYPGAHTTTIKYTSALTYDAVQVMTEAFRNLRKQRIEISRRGNAGDCLANPAVPWGQGVEIERALKQVQVEGLSGNIKFDQNGKRINYTINIMELKTNGPRKIGYWSEVDKMVVTLTELPSGNDTSGLENKTVVVTTILESPYVMMKKNHEMLEGNERYEGYCVDLAAEIAKHCGFKYKLTIVGDGKYGARDADTKIWNGMVGELVYGKADIAIAPLTITLVREEVIDFSKPFMSLGISIMIKKPQKSKPGVFSFLDPLAYEIWMCIVFAYIGVSVVLFLVSRFSPYEWHTEEFEDGRETQSSESTNEFGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKI

SEQ ID NO: 14 - protein, 908 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 15 - protein, 1328 aa, Homo sapiens
MSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSIDLQSFMARGLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDRVKEYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQERQLPPLGPTNPRVTLAPPWNGLAPPAPPPPPRLQITENGEFRNTADH

SEQ ID NO: 16 - protein, 14 aa, Homo sapiens
MSYAPFRDVRGPSM

SEQ ID NO: 17 - protein, 18 aa, Homo sapiens
SPYDRPGWNPRFCIISGN

SEQ ID NO: 18 - protein, 32 aa, Homo sapiens
KSVPGGKQYSMEGAPAAPFRPSQGFLSRRLKS

SEQ ID NO: 19 - protein, 11 aa, Homo sapiens
RTSSFRQILPR

SEQ ID NO: 20 - protein, 5 aa, Artificial sequence
  Synthetic peptide
CSQLS

SEQ ID NO: 21 - protein, 10 aa, Artificial sequence
  Synthetic peptide
GSAAVAAIFG

SEQ ID NO: 22 - protein, 9 aa, Homo sapiens
SSILGQEFC

SEQ ID NO: 23 - protein, 49 aa, Homo sapiens
KDNSRRVDNVLKLWIIEARELPPKKRYYCELCLDDMLYARTTSKPRSAS

SEQ ID NO: 24 - protein, 8 aa, Homo sapiens
NLPAVRAL

SEQ ID NO: 25 - protein, 4 aa, Homo sapiens
YDSD

SEQ ID NO: 26 - protein, 12 aa, Homo sapiens
KRKKDKAGYVGL

SEQ ID NO: 27 - protein, 28 aa, Homo sapiens
SGGGGGSGGGSGGKGKGGCPAVRLKARY

SEQ ID NO: 28 - protein, 4 aa, Artificial sequence
  Synthetic peptide
GGKE

SEQ ID NO: 29 - protein, 4 aa, Homo sapiens
YHYI

SEQ ID NO: 30 - protein, 7 aa, Artificial sequence
  Synthetic peptide
SGVNRFM

SEQ ID NO: 31 - protein, 7 aa, Artificial sequence
  Synthetic peptide
ENTLVSK

SEQ ID NO: 32 - protein, 4 aa, Homo sapiens
YDAV

SEQ ID NO: 33 - protein, 13 aa, Artificial sequence
  Synthetic peptide
EGREDIADRLLSG

SEQ ID NO: 34 - protein, 5 aa, Homo sapiens
KIGTW

SEQ ID NO: 35 - protein, 4 aa, Homo sapiens
KFTS

SEQ ID NO: 36 - protein, 6 aa, Homo sapiens
VTTILE

SEQ ID NO: 37 - protein, 9 aa, Homo sapiens
RFEGYCIDL

SEQ ID NO: 38 - protein, 5 aa, Homo sapiens
RELST

SEQ ID NO: 39 - protein, 9 aa, Homo sapiens
PQLSKEALV

SEQ ID NO: 40 - protein, 8 aa, Artificial sequence
  Synthetic peptide
DGKYGRLA

SEQ ID NO: 41 - protein, 9 aa, Artificial sequence
  Synthetic peptide
WNPGMVREL

SEQ ID NO: 42 - protein, 23 aa, Artificial sequence
  Synthetic peptide
VREEVIDFSKRDLNSSIDLQPFM

SEQ ID NO: 43 - protein, 15 aa, Artificial sequence
  Synthetic peptide
LGISSSMDMARLPSI

SEQ ID NO: 44 - protein, 12 aa, Artificial sequence
  Synthetic peptide
PGGGKDLVFSFL

SEQ ID NO: 45 - protein, 5 aa, Homo sapiens
IWMYI

SEQ ID NO: 46 - protein, 8 aa, Homo sapiens
SRFSPYEW

SEQ ID NO: 47 - protein, 6 aa, Homo sapiens
NSLWFS

SEQ ID NO: 48 - protein, 4 aa, Homo sapiens
MQQG

SEQ ID NO: 49 - protein, 5 aa, Homo sapiens
RIVGG

SEQ ID NO: 50 - protein, 25 aa, Homo sapiens
WWFFTLIIISSYTANLAAFLTVERM

SEQ ID NO: 51 - protein, 68 aa, Artificial sequence
  Synthetic peptide
YGPPGGHGGGGGHGPPSSHHHHHHHHHHRGGEPPGDTFAPFHGYSKSEDLSSGVPKPPAASILHSHSY

SEQ ID NO: 52 - protein, 5 aa, Artificial sequence
  Synthetic peptide
RSKIS

SEQ ID NO: 53 - protein, 4 aa, Homo sapiens
DKMW

SEQ ID NO: 54 - protein, 34 aa, Artificial sequence
  Synthetic peptide
AEGGPGGGSGGGSGGGGGGQPPPLQRGKSQQLTV

SEQ ID NO: 55 - protein, 5 aa, Homo sapiens
LLEST

SEQ ID NO: 56 - protein, 10 aa, Artificial sequence
  Synthetic peptide
IDSKGGSGYG

SEQ ID NO: 57 - protein, 5 aa, Artificial sequence
  Synthetic peptide
GRVKL

SEQ ID NO: 58 - protein, 6 aa, Homo sapiens
EFLYKS

SEQ ID NO: 59 - protein, 67 aa, Homo sapiens
HPAMAEPLPEPKKRLLDAQERQLPPLGPTNPRVTLAPPWNGLAPPAPPPPPRLQITENGEFRNTADH